(19) United States
(12) Patent Application Publication
(10) Pub. No.: US 2023/0346764 A1
(43) Pub. Date: Nov. 2, 2023

(54) Methods and Compositions for Treating Pruritus, Xerosis, and Associated Disease Using CCR3-Inhibitors

(71) Applicant: Alkahest, Inc., San Carlos, CA (US)

(72) Inventors: Steven P. Braithwaite, Redwood City, CA (US); S. Sakura Minami, San Francisco, CA (US); Arnaud E.J. Teichert, San Francisco, CA (US)

(21) Appl. No.: 18/119,522

(22) Filed: Mar. 9, 2023

Related U.S. Application Data
(63) Continuation of application No. 16/159,048, filed on Oct. 12, 2018, now abandoned.
(60) Provisional application No. 62/572,251, filed on Oct. 13, 2017.

(51) Int. Cl. A61K 31/4545; A61K 9/14; A61K 9/00; A61K 9/28; A61K 9/06; A61K 9/08; A61K 9/12; A61P 17/00; A61K 9/20; A61K 9/48

(52) U.S. Cl. CPC A61K 31/4545; A61K 9/0014; A61K 9/0019; A61K 9/0053; A61K 9/0075; A61K 9/008; A61K 9/06; A61K 9/08; A61K 9/12; A61K 9/14; A61K 9/145; A61K 9/2009; A61K 9/2013; A61K 9/2018; A61K 9/2027; A61K 9/2054; A61K 9/2059; A61K 9/2853; A61K 9/2866; A61K 9/4858; A61P 17/00

(57) ABSTRACT

Methods of treating symptoms of skin disorders with CCR3 modulating agents are provided. The methods include administering a therapeutically effective amount of the CCR3 modulating agent to the subject, with a concomitant improvement in pruritis, xerosis, or other skin disorder-affected function. Skin disorders upon which the methods of the invention can improve symptoms and causes of the disorders include eczema, bullous pemphigoid, atopic dermatitis, and psoriasis.

### I. CROSS-REFERENCE TO RELATED APPLICATION

Pursuant to 35 U.S.C. § 119 (e), this application claims priority to the filing date of U.S. Provisional Pat. Application No. 62/572,251, filed Oct. 13, 2017; the disclosure of which application is herein incorporated by reference.

### II. FIELD OF THE INVENTION

This invention pertains to the prevention and treatment of skin disorders, e.g. pruritis and xerosis and associated disease. In particular, the invention relates to the use of CCR3 modulating agents, such as CCR3 inhibitors, to treat and/or prevent disorders associated with the skin.

### III. INTRODUCTION

The following is offered as background information only and is not admitted being prior art to the present invention.

Eosinophil-associated rare diseases are a group of uncommon conditions in which eosinophil leukocytes play a critical pathophysiological role. The skin is one site at which eosinophils can become pathologically upregulated, contributing to a broad spectrum of infectious, allergic, autoimmune, and neoplastic skin diseases such as atopic dermatitis, psoriasis, and pemphigoid disorders (Roth N, et al., Allergy, 66(11):1477-86 (2011)). Despite the known abundance of these cells in disease, very little is known about the pathophysiologic mechanism underlying eosinophilic pathology in the skin. For most of these rare eosinophilic skin diseases, the causes and pathogenic mechanisms remain largely unknown, and further investigations are needed for advances in clinical diagnosis and devising of effective treatments (Long H, et al., Clin Rev Allergy Immunol, 50(2):189-213 (2016)).

Bullous pemphigoid (BP) is the most common autoimmune, sub-epidermal blistering disease of the skin. Eosinophil infiltration is a prominent feature of BP and they can be located in the upper dermis, often lining the dermal-epidermal junction. They are also found in blisters associated with BP, and their presence is in part what distinguishes BP from other blistering dermatoses (Lever WF, AMA Arch Derm Syphiol, 64(6):727-53 (1951) and Eng AM, et al., Arch Dermatol. 110(1):51-7 (1974)). In skin lesions, mainly hypodense eosinophils are observed suggesting an activated state (Tsuda S, et al., J Dermatol. 19(5):270-79 (1992)). Such activated eosinophils are usually located in the basement membrane zone and show degranulation on keratinocytes (Engmann J, et al., Acta Derm Venereol. 97(4):464-71 (2017)). Eotaxins, in particular CC chemokine ligand (CCL)11 (also known as Eotaxin-1), as well as its principal receptor, C-C Motif Chemokine Receptor 3 (CCR3), have been demonstrated in BP lesions (Frezzolini A, et al., Eur J Dermatol. 12(1):27-31 (2002)). Moreover, Eotaxin is strongly expressed by keratinocytes around blisters and has been detected at high levels in blister fluids correlating with the number of tissue eosinophils (Wakugawa M, et al., Br J Dermatol. 143(1):112-16 (2000)).

Severe pruritis and blisters are observed in virtually all patients. (JAMA Dermatol, 49(3): 382 (2013)). Standard of care for BP consists of topical or oral corticosteroids including topical clobetasol, topical betamethasone, topical mometasone furoate, and oral prednisone. (Zhao CY, et al., F1000Research 2015, 4(F1000 Faculty Rev): 1313). Oral corticosteroids at high doses are poorly tolerated particularly in the elderly however. (Joly P, et al., Drugs Aging 22(7):571-76 (2005)). Additionally, oral corticosteroids may contribute to high rates of mortality. (Id.) And topical corticosteroids must be administered over the entire body with wraps, increasing burden on patients and caregivers as well as reducing compliance. Moreover, BP can also be resistant to corticosteroids, necessitating new agents with different alternate of action.

Eosinophils are also a significant component of atopic dermatitis (AD), a chronic inflammatory skin disease with specific immune and inflammatory mechanisms. The role of eosinophils in AD has been suggested by the presence of eosinophilia in AD patients and eosinophil infiltrates in AD lesions (Liu FT, et al., Clin Rev Allergy Immunol. 41(3):298-310 (2011)). Moreover, patient eosinophil blood levels (Kägi MK, et al., Dermatology. 185(2):88-92 (1992)) as well as eosinophil-specific granule proteins levels in blood (Leiferman KM, J Am Acad Dermatol. (6 Pt 2):1101-12 (1991)) correlate with AD disease activity. Peripheral blood eosinophilia has been proposed as a diagnostic tool in differentiating atopic AD from non-atopic AD (Nishimoto M, et al., Arerugi. 47(6):591-6 (1998)), suggesting that therapeutics targeting eosinophils could be particularly effective in at least some subsets of AD patients.

Local neurogenic inflammation is a key component of AD (Misery L, Clin Rev Allergy Immunol. 41(3):259-66 (2011)), and pruritis (itch) in general is a significant untreated symptom of BP, with some observed parallels between BP and AD patients (Kulczyck-Siennicka L, Biomed Res Int. 5965492 (2017)). Since eosinophils have been shown to localize to nerves in inflammatory bowel diseases (Smyth CM, et al., PLoS One. 8(5):e64216 (2013)), eosinophils are potential key players in the progression and sustenance of diseases involving chronic local inflammation. Moreover, eosinophilia-associated neuropathy has been shown to be associated with skin denervation (Chao CC, et al., Arch Neurol. 64(7):959-65 (2007)). Multiple molecular pathways have been shown to overlap between eosinophils and nerves. For example, the neuropeptide Substance P (SP) is secreted by both nerves and eosinophils (Akiyama T, et al., Pain. 155(1):80-92 (2014)) while eosinophils of AD patients stimulated in vitro elaborate large amounts of BDNF (Raap U, et al., J Allergy Clin Immunol. 115(6):1268-75 (2005)), a neurotrophic factor known to promote neurons maturation and proliferation. Furthermore, human eosinophils produce neurotrophins and secrete NGF under neurological stimuli, a neurotrophic factor for sensory neurons, which may contribute to the intensification of the neural response in AD patients (Kobayashi H, et al., Blood. \99(6):2214-20 (2002)).

Various mouse models recapitulate certain features of BP, particularly the sub-epidermal blistering, complement activation, mast cell degranulation, neutrophil infiltration and proteinase secretion, but not the eosinophil component of BP (Heimbach L, et al., G Ital Dermatol Venereol. 144(4):423-31 (2009) and Ujiie H, et al., J Immunol. 184(4):2166-74 (2010)). This lack of eosinophil contribution along with the relative brevity (a few days on the whole) of those mouse models do not provide an appropriate pre-clinical tool to investigate BP per se. Nevertheless, other approaches, such as topical exposure to the skin sensitizer, Oxazolone, can trigger eosinophil recruitment, itch and skin neuropathy (Haoli J, et al., J Invest Dermatol. 129(1): 31-40 (2009) and Liu B, et al., FASEB J. 27(9):3549-63 (2013)) providing a pre-clinical model to investigate potential eosinophil-based therapeutics’ efficacy on key features of BP, AD, and other eosinophil-related cutaneous diseases. As such the use of targeted approaches to recreate specific features of these diseases in a pre-clinical model can help determine the efficacy of eosinophil-based therapeutics against symptoms such as eosinophil recruitment, pruritis, xerosis, and skin neuropathy.

Previous work has shown that CCR3 plays a central role in eosinophil recruitment to the skin (Sénéchal S, et al., Lab Invest. 82(7):929-39 (2002)) and in chronic inflammation (Fulkerson, PC, et al., Proc Natl Acad Sci USA. 103(44): 16418-23 (2006)). As such, the compounds of the invention such as Compound 1 and its analogues, which are small molecule antagonists of CCR3, are effective new therapeutic intervention targeting eosinophils through the eotaxin/CCR3 pathway for Bullous Pemphigoid and other cutaneous diseases involving eosinophils.

### IV. INCORPORATION BY REFERENCE

All publications, patents, and patent applications mentioned in this specification are herein incorporated by reference to the same extent as if each individual publication or patent application was specifically and individually indicated to be incorporated by reference.

### V. SUMMARY

Current treatments for pathologic pruritis, xerosis and associated disease have been based on treating the symptoms, but not the root cause(s) of the disease. Additionally, these current treatments have exhibited limitations such as unwanted side effects, drug tolerance, and limited efficacy. The present invention overcomes these drawbacks in part because the compounds of the invention target a pathway unrelated to those targeted by current treatments. Also, for example Compound 1, a compound of the invention, can be administered systemically (e.g. PO), not only targeting symptoms occurring directly at the skin, but systemically through inhibition of the mechanisms (e.g. eosinophil recruitment through activation of the Eotaxin-1/CCR3 pathway) that are a cause those symptoms. Moreover, Compound 1 can also be formulated into a topical agent for immediate relief of symptoms where they occur in the skin.

The compounds of the invention act as antagonists of c-c motif chemokine receptor 3 (CCR3), the receptor for Eotaxin-1. Eotaxin-1 (CCL11) is a protein that is increased in levels in blood plasma with aging, which is one of the factors implicated with increased pruritis and xerosis. (Villeda et al., The aging systemic milieu negatively regulates neurogenesis and cognitive function, Nature, 477(7362):90-94 (2011), herein incorporated by reference). Eotaxin/CC11 acts primarily on the G-protein coupled receptor CCR3 which is expressed on eosinophils in the periphery and on neurons and glial cells in the central nervous system. (Xia, M, et al., Immunohistochemical Study of the β-Chemokine Receptors CCR3 and CCR5 and Their Ligands in the Normal and Alzheimer’s Disease Brains, Am. J. Pathol. 153(1);31-37 (1998)).

Methods of treating patients for symptoms such as pruritis and xerosis associated with dermatologic diseases are provided, including by way of example and not limitation, xerosis, dermatitis, dyshydrotic dermatitis, drug reactions, urticaria, atopic dermatitis/neurodermatitis, seborrheic dermatitis, psoriasis, palmoplantar pustulosis, lichen planus, pityriasis rubra pilaris, darier disease, Hailey-Hailey disease, Grover’s disease, polymorphic light eruptions, bullous pemphigoid, acquired epidermolysis bullosa, dermatitis herpetiformis, pemphigus vulgaris, dermatomyositis, systemic sclerosis, Sjögren syndrome, Herpes simplex, Herpes zoster, tineas, candidal intertrigo; malassezia folliculitis, Ofuji’s disease, scabies, lice, cutaneous larva migrans, insect bites/arthropod reactions, rosacea, mastocytosis, cutaneous lymphomas, mycosis fungoides, and Sezary syndrome, and the like. Other aspects of the methods include treatment of symptoms of systemic diseases manifesting pruritic and xerosis symptoms including by way of example and not limitation, Liver diseases (primary biliary cirrhosis, primary sclerosing cholangitis, extrahepatic cholestasis, Hepatitis B and C); Kidney diseases (chronic kidney insufficiency); Hematologic diseases (polycythemia vera, Hodgkin disease, Non-Hodgkin lymphomas, leukemias, myeloma multiplex, iron deficiency, systemic mastocytosis, hypereosinophilic syndrome, myelodysplastic syndromes); Endocrine disorders (hyperthyroidism, hypothyroidism, hyperparathyroidism, diabetes); Neurologic diseases (neuropathic pruritus); Brain injury/tumor (unilateral pruritus); sclerosis multiplex; small fiber neuropathy; solid tumors (paraneoplastic pruritus); carcinoid syndrome; and infectious diseases (HIV infection/AIDS, infestations). Aspects of the methods include modulation of CCR3, the principal receptor of CCL11/eotaxin-1 through the administration of a therapeutically effective amount of CCR3 antagonists of the invention. The methods include administering an effective therapeutic dose of CCR3 antagonists to subjects or patients as well as monitoring for specific clinical endpoints such as improvement in skin dryness and cessation of scratching due to itch.

### VI. BRIEF DESCRIPTION OF THE FIGURES

FIG. 1 shows that Compound 1 is efficacious at decreasing ovalbumin (OVA)-induced pulmonary eosinophil influx in a human CCR3 knock-in Balb/c mouse model. Mice challenged with OVA were administered a dose range of Compound 1 from 1 to 100 mg/kg. Compound 1 exhibited a dose-dependent relationship with respect to inhibiting eosinophil influx.

FIG. 2 depicts the inhibition of OVA-induced pulmonary eosinophil influx by Compound 1 in a human CCR3 knock-in mouse model, with measured IC₅₀ concentration (i.e. noted as ID₅₀ in FIG. 2). Compound 1 inhibited OVA-induced pulmonary eosinophilic inflammation in a dose-dependent manner, with an IC₅₀ of 4.9 mg/kg.

FIG. 3 depicts the percentage of inhibition of eosinophil shape change (ESC) in human whole blood. Compound 1 exhibited dose-dependent inhibition of ESC induced by eotaxin-1 incubation of whole blood from Compound 1-treated patients, using flow cytometry to determine size and granularity of eosinophils.

FIG. 4 depicts the percentage of inhibition of CCR3 internalization in human whole blood. Compound 1 exhibited dose-dependent inhibition of CCR3 internalization induced by eotaxin-1 incubation of whole blood from Compound 1-treated patients, using flow cytometry to determine internalization.

FIG. 5 depicts the results of an “oxazolone model of chronic skin inflammation.” A time-dependent increase in skin eosinophil levels in mice treated with a topical application of Oxazolone was observed. Oxazolone was administered topically to 8-week-old male SKH-1 Elite hairless mice at 5% concentration for sensitization. Subsequently, chronic inflammation was triggered 7 days after oxazolone sensitization, with the mice treated topically every other day with oxazolone (dose range 0.1 to 0.5%) on both flanks until the end of the study. Levels of eosinophils in the skin of the mice were determined and plotted over time.

FIG. 6 depicts the results of an “oxazolone model of chronic skin inflammation.” A time-dependent increase in blood eosinophil levels in mice treated with a topical application of Oxazolone was observed. Oxazolone was administered topically to 8-week-old male SKH-1 Elite hairless mice at 5% concentration for sensitization. Subsequently, chronic inflammation was triggered 7 days after oxazolone sensitization, with the mice treated topically every other day with oxazolone (dose range 0.1 to 0.5%) on both flanks until the end of the study. Levels of eosinophils in the blood of the mice were determined and plotted over time.

FIG. 7 reports the effects of dexamethasone and Compound 1 on a skin scaling/dryness visual scoring assay. SKH-1 Elite mice were sensitized using the oxazolone model of chronic skin inflammation with an initial 5% oxazolone topical concentration. To trigger chronic inflammation, mice were administered 0.1% oxazolone topically every other day on both flanks until the end of the study. By day 17 both Compound 1 and dexamethasone showed efficacy at reducing skin dryness, with Compound 1 exhibiting a trend toward faster recovery compared to dexamethasone.

FIG. 8 reports the effects of Compound 1 and dexamethasone on the blood eosinophil levels of oxazolone-treated mice. Mice sensitized with the topical oxazolone model of chronic skin inflammation received Compound 1 orally (treated immediately or delayed after oxazolone administration), dexamethasone, or compound 1 and dexamethasone. Compound 1 alone returned eosinophil levels to levels similar to control mice, whereas dexamethasone resulted in a more severe reduction of eosinophil levels.

FIGS. 9A and 9B report the effects on blood lymphocyte (FIG. 9A) and white blood cell (WBC) (FIG. 9B) levels in mice treated as in FIG. 8. Lymphocyte level reduction was severe with dexamethasone treated mice and less so with Compound 1 treatment. This, in conjunction with FIG. 8, shows that Compound 1 is more discriminate than dexamethasone in reduction of blood cell types levels, which supports the association of Compound 1 with less-severe adverse reactions.

FIGS. 10A, 10B and 10C report the effects of Compound 1 (Cmpd 1) on certain blood plasma cytokine levels. Levels of tumor necrosis factor alpha (TNFα) (FIG. 10A), interleukin 6 (FIG. 10B), and interleukin-1 beta (IL1β) (FIG. 10C) were all decreased with Compound 1 treatment.

FIGS. 11A and 11B report the effects of Compound 1 (Cmpd 1) on bullous pemphigoid targets, interleukin-5 (IL5) (FIG. 11A) and interleukin-17 (IL17) (FIG. 11B). Both cytokines were decreased in blood plasma of Compound 1-treated mice.

### VII. DETAILED DESCRIPTION

Aspects of the invention include methods of treating skin-disorders and corresponding symptoms such as pruritis and xerosis. The skin-disorders and corresponding symptoms may manifest themselves as pruritis and xerosis associated with dermatologic diseases including by way of example and not limitation, xerosis, dermatitis, dyshydrotic dermatitis, drug reactions, urticaria, atopic dermatitis/neurodermatitis, seborrheic dermatitis, psoriasis, palmoplantar pustulosis, lichen planus, pityriasis rubra pilaris, darier disease, Hailey-Hailey disease, Grover’s disease, polymorphic light eruptions, bullous pemphigoid, acquired epidermolysis bullosa, dermatitis herpetiformis, pemphigus vulgaris, dermatomyositis, systemic sclerosis, Sjögren syndrome, Herpes simplex, Herpes zoster, tineas, candidal intertrigo; malassezia folliculitis, Ofuji’s disease, scabies, lice, cutaneous larva migrans, insect bites/arthropod reactions, rosacea, mastocytosis, cutaneous lymphomas, mycosis fungoides, and Sezary syndrome, and the like. Other aspects of the methods include treatment of symptoms of systemic diseases manifesting pruritic and xerosis symptoms including by way of example and not limitation, Liver diseases (primary biliary cirrhosis, primary sclerosing cholangitis, extrahepatic cholestasis, Hepatitis B and C); Kidney diseases (chronic kidney insufficiency); Hematologic diseases (polycythemia vera, Hodgkin disease, Non-Hodgkin lymphomas, leukemias, myeloma multiplex, iron deficiency, systemic mastocytosis, hypereosinophilic syndrome, myelodysplastic syndromes); Endocrine disorders (hyperthyroidism, hypothyroidism, hyperparathyroidism, diabetes); Neurologic diseases (neuropathic pruritus); Brain injury/tumor (unilateral pruritus); sclerosis multiplex; small fiber neuropathy; solid tumors (paraneoplastic pruritus); carcinoid syndrome; and infectious diseases (HIV infection/AIDS, infestations).

Other aspects of the invention include methods of treating pruritis and xerosis that are symptoms of systemic disease. This includes by way of example and not limitation, liver diseases (primary biliary cirrhosis, primary sclerosing cholangitis, extrahepatic cholestasis, Hepatitis B and C); Kidney diseases (chronic kidney insufficiency); Hematologic diseases (polycythemia vera, Hodgkin disease, Non-Hodgkin lymphomas, leukemias, myeloma multiplex, iron deficiency, systemic mastocytosis, hypereosinophilic syndrome, myelodysplastic syndromes); Endocrine disorders (hyperthyroidism, hypothyroidism, hyperparathyroidism, diabetes); Neurologic diseases (neuropathic pruritus); Brain injury/tumor (unilateral pruritus); sclerosis multiplex; small fiber neuropathy; solid tumors (paraneoplastic pruritus); carcinoid syndrome; and infectious diseases (HIV infection/AIDS, infestations).

Other aspects of the invention include modulation of CCR3, the principal receptor of CCL11/eotaxin-1 through the administration of a therapeutically effective amount of CCR3 antagonists of the invention. The methods include administering an effective therapeutic dose of CCR3 antagonists to subjects or patients as well as monitoring for specific clinical endpoints such as improvement in skin dryness and cessation of scratching due to itch. The methods of monitoring for specific clinical endpoints, include for example, observation of skin dryness based on a graduated scale (e.g. 0 through 4) where zero is absence of dryness and 4 is extreme dryness. The methods of monitoring for specific clinical endpoints, also include for example, observation of cessation or decreased scratching in response to pruritis or itch, observation of decreased damage to skin due to scratching, or other such methods of monitoring changes in scratching.

By “treatment” it is meant that at least an amelioration of one or more symptoms associated with a skin disorder afflicting the subject is achieved, where amelioration is used in a broad sense to refer to at least a reduction in the magnitude of a parameter, e.g., a symptom associated with the indication being treated. As such, treatment also includes situations where a pathological condition, or at least symptoms associated therewith, are completely inhibited, e.g., prevented from happening, or stopped, e.g., terminated, such that the subject no longer suffers from the impairment, or at least the symptoms that characterize the impairment. In some instances, “treatment”, “treating” and the like refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” may be any treatment of a disease in a subject, and includes: (a) preventing the disease from occurring in a subject which may be predisposed to the disease but has not yet been diagnosed as having it; (b) inhibiting the disease, i.e., arresting its development; (c) relieving the disease, i.e., causing regression of the disease; or (d) preventing relapse of the disease. Treatment may result in a variety of different physical manifestations, e.g., modulation in gene or protein expression, decreased itch sensation, decreased skin dryness, etc. Treatment of ongoing disease, where the treatment stabilizes or reduces the undesirable clinical symptoms of the patient, occurs in some embodiments. Such treatment may be performed prior to complete loss of function in the affected tissues. The subject therapy may be administered during the symptomatic stage of the disease, and in some cases after the symptomatic stage of the disease.

Other aspects of the invention include administration of oral forms of the compounds of invention, including by tablet form, spray, or gavage. Other aspects of the invention include administration of the compounds of the invention in intra venous form, or through administration of topical forms of the compounds of the invention.

Additional aspects of the invention include diagnosing or monitoring the severity or progression of pruritis or xerosis-related disease. By way of example and not limitation, such diagnosing or monitoring may be performed by determining the expression, concentration, or presence of eosinophil cationic protein (ECP), which is a predictive marker of bullous pemphigoid severity and outcome. (Giusti D, et al., Nature Scientific reports, 7:4833 (2017)). Also by way of example and not limitation, such diagnosing or monitoring may be performed by determining the expression, concentration, or presence of Interleukin-31 (IL-31) which exists in high concentrations in patients with BP compared with healthy controls. (Rüdrich U, et al., Acta Derm Venereol, 98(8):766-71 (2018)).

Before the present methods and compositions are described, it is to be understood that this invention is not limited to a particular method or composition described, and as such may, of course, vary. It is also to be understood that the terminology used herein is for the purpose of describing particular embodiments only, and is not intended to be limiting, since the scope of the present invention will be limited only by the appended claims.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention. “Between,” when used in the context of a numerical range, includes all numbers within the range including the upper and lower limits unless the context clearly dictates otherwise.

It is noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those having skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### A. Compounds

The methods of the invention further comprise administration to a subject of the compounds that follow. In the groups, radicals, or moieties defined in this “Compounds” section, the number of carbon atoms is often specified preceding the group, for example, C₁₋₆ alkyl means an alkyl group or radical having 1 to 6 carbon atoms. In general, for groups comprising two or more subgroups which are disclosed in this “Compounds” section, the last named group is the radical attachment point, for example, “thioalkyl” means a monovalent radical of the formula HS-Alk-. Unless otherwise specified below, conventional definitions of terms control and conventional stable atom valences are presumed and achieved in all formulas and groups.

An embodiment of the invention further comprises administration to a subject of the compounds of formula 1, wherein

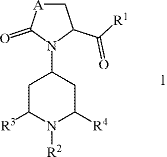

A is CH₂, O or N-C₁₋₆-alkyl;
R¹ is selected from
   NHR^{1.1}, NMeR^{1.1}.,
   NHR^{1.2}, NMeR^{1.2};
   NHCH₂-R^{1.3};
   NH-C₃₋₆-cycloalkyl, whereas optionally one carbon atom is replaced by a nitrogen atom, whereas the ring is optionally substituted with one or two residues selected from the group consisting of C₁₋₆-alkyl, O-C₁₋₆-alkyl, NHSO₂-phenyl, NHCONH-phenyl, halogen, CN, SO₂-C₁₋₆-alkyl, COO-C₁₋₆-alkyl;
   a C₉ ₒᵣ ₁₀-bicyclic-ring, whereas one or two carbon atoms are replaced by nitrogen atoms and the ring system is bound via a nitrogen atom to the basic structure of formula 1 and whereas the ring system is optionally substituted with one or two residues selected from the group consisting of C₁₋₆-alkyl, COO-C₁₋₆-alkyl, C₁₋₆-haloalkyl, O-C₁₋₆-alkyl, NO₂, halogen, CN, NHSO₂-C₁₋₆-alkyl, methoxy-phenyl;
   a group selected from NHCH(pyridinyl)CH₂COO-C₁₋₆-alkyl, NHCH(CH₂O-C₁₋₆-alkyl)-benzoimidazolyl, optionally substituted with halogen or CN;
   or 1-aminocyclopentyl, optionally substituted with methyl-oxadiazole
   R^{1.1} is phenyl, optionally substituted with one or two residues selected from the group consisting of C₁₋₆-alkyl, C₂₋₆-alkenyl, C₂₋₆-alkynyl, C₁₋₆-haloalkyl, C₁₋₆-alkylene-OH, C₂₋₆-alkenylene-OH, C₂₋₆-alkynylene-OH, CH₂CON(C₁₋₆-alkyl)₂, CH₂NHCONH-C₃₋₆-cycloalkyl, CN, CO-pyridinyl, CONR^{1.1.1}_{R}^{1.1.2}, COO-C₁₋₆-alkyl, N(SO₂-C₁₋₆-alkyl)(CH₂CON(C₁₋₄-alkyl)₂) O-C₁₋₆-alkyl, O-pyridinyl, SO₂-C₁₋₆-alkyl, SOa-C₁₋₆-alkylen-OH, SO₂-C₃₋₆-cycloalkyl, SO₂-piperidinyl, SO₂NH-C₁₋₆-alkyl, SO₂N(C₁₋₆-alkyl)₂, halogen, CN, CO-morpholinyl, CH₂-pyridinyl or a heterocyclic ring optionally substituted with one or two residues selected from the group consisting of C₁₋₆-alkyl, NHC₁₋₆-alkyl and =0;
      R^{1.1.1} H, C₁₋₆-alkyl, C₃₋₆-cycloalkyl, C₁₋₆-haloalkyl, CH₂CON(C₁₋₆-alkyl,)₂, CH₂CO-azetindinyl, C₁₋₆-alkylen-C₃₋₆-cycloalkyl, CH₂-pyranyl, CH₂-tetrahydrofuranyl, CH₂-furanyl, C₁₋₆-alkylen-OH or thiadiazolyl, optionally substituted with C₁₋₆-alkyl;
      R^{1.1.2} H, C₁₋₆-alkyl, SO₂C₁₋₆-alkyl;
      or R^{1.1.1} and R^{1.1.2} together are forming a four-, five- or six-membered carbocyclic ring, optionally containing one N or O, replacing a carbon atom of the ring, optionally substituted with one or two residues selected from the group consisting of C₁₋₆-alkyl, C₁₋₄-alkylene-OH, OH, =O; or
   R^{1.1} is phenyl, wherein two adjacent residues are together forming a five- or six-membered carbocyclic aromatic or non-aromatic ring, optionally containing independently from each other one or two N, S, or SO₂, replacing a carbon atom of the ring, wherein the ring is optionally substituted with C₁-₄-alkyl or =O;
   R^{1.2} is selected from
      heteroaryl, optionally substituted with one or two residues selected from the group consisting of C₁₋₆-alkyl, C₂₋₆-alkenyl, C₂₋₆-alkynyl, C₃₋₆-cycloalkyl, CH₂COO-C₁₋₆-alkyl, CONR^{1.2.1}R^{1.2.2}, COR^{1.2.3}, COO-C₁₋₆-alkyl, CONH₂, O-C₁₋₆-alkyl, halogen, CN, SO₂N(C₁₋₆-alkyl)₂ or heteroaryl optionally substituted with one or two residues selected from the group consisting of C₁₋₆-alkyl;
      heteroaryl, optionally substituted with a five- or six-membered carbocyclic non-aromatic ring containing independently from each other two N, O, S, or SO₂, replacing a carbon atom of the ring;
      a aromatic or non-aromatic C₉ ₒᵣ ₁₀-bicyclic-ring, whereas one or two carbon atoms are replaced by N, O or S each optionally substituted with one or two residues selected from the group consisting of N(C₁₋₆-alkyl)₂, CONH-C₁₋₆-alkyl, ═O;
      a heterocyclic non-aromatic ring, optionally substituted with pyridinyl;
      4,5-dihydro-naphtho[2,1-d]thiazole, optionally substituted with NHCO-C₁₋₆-alkyl,
      R^{1.2.1} H, C₁₋₆-alkyl, C₁₋₆-alkylene-C₃₋₆-cycloalkyl, C₁₋₄-alkylene-phenyl, C ₁₋₄-alkylene-furanyl, C₃₋₆-cycloalkyl, C₁₋₄-alkylene-O-C₁₋₄-alkyl, C₁₋₆-haloalkyl or a five- or six-membered carbocyclic non-aromatic ring, optionally containing independently from each other one or two N, O, S, or SO₂, replacing a carbon atom of the ring, optionally substituted with 4-cyclopropylmethyl-piperazinyl
      R^{1.2.2} H, C₁₋₆-alkyl;
      R^{1.2.3} a five- or six-membered carbocyclic non-aromatic ring, optionally containing independently from each other one or two N, O, S, or SO₂, replacing a carbon atom of the ring;
   R^{1.3} is selected from phenyl, heteroaryl or indolyl, each optionally substituted with one or two residues selected from the group consisting of C₁₋₆-alkyl, C₃₋₆-cycloalkyl, O-C₁₋₆-alkyl, O-C₁₋₆-haloalkyl, phenyl, heteroaryl;
R² is selected from the group consisting of C₁₋₆-alkylene-phenyl, C₁₋₆-alkylene-naphthyl, and C₁₋₆-alkylene-heteroaryl; each optionally substituted with one, two or three residues selected from the group consisting of C₁₋₆-alkyl, C₁₋₆-haloalkyl, O-C₁₋₆-alkyl, O-C₁₋₆-haloalkyl, halogen;
R³ is H, C₁₋₆-alkyl;
R⁴ is H, C₁₋₆-alkyl;
   or R³ and R⁴ together are forming a CH₂—CH₂ group.

Another embodiment of the present invention further comprises administration to a subject of the compounds of formula 1 (above), wherein
A is CH₂, O or N-C₁₋₄-alkyl;
R¹ is selected from
   NHR^{1.1}, NMeR^{1.1};
   NHR^{1.2}, NMeR^{1.2};
   NHCH₂-R^{1.3};
   R^{1.1} is phenyl, optionally substituted with one or two residues selected from the group consisting of C₁₋₆-alkyl, C₂₋₆-alkenyl, C₂₋₆-alkynyl, C₁₋₆-haloalkyl, C₁₋₆-alkylene-OH, C₂₋₆-alkenylene-OH, C₂₋₆-alkynylene-OH, CH₂CON(C₁₋₆-alkyl)₂, CH₂NHCONH-C₃₋₆-cycloalkyl, CN, CO-pyridinyl, CONR^{1.1.1}_{R}^{1.1.2}, COO-C₁₋₆-alkyl, N(SO₂-C₁₋₆-alkyl)(CH₂CON(C₁₋₄-alkyl)₂) O-C₁₋₆-alkyl, O-pyridinyl, SO₂-C₁₋₆-alkyl, SOa-C₁₋₆-alkylen-OH, SO₂-C₃₋₆-cycloalkyl, SO₂-piperidinyl, SO₂NH-C₁₋₆-alkyl, SO₂N(C₁₋₆-alkyl)₂, halogen, CN, CO-morpholinyl, CH₂-pyridinyl or a heterocyclic ring optionally substituted with one or two residues selected from the group consisting of C₁₋₆-alkyl, NHC₁₋₆-alkyl,, =O;
      R^{1.1.1} H, C₁₋₆-alkyl, C₃₋₆-cycloalkyl, C₁₋₆-haloalkyl, CH₂CON(C₁₋₆-alkyl,)₂, CH₂CO-azetindinyl, C₁₋₆-alkylen-C₃₋₆-cycloalkyl, CH₂-pyranyl, CH₂-tetrahydrofuranyl, CH₂-furanyl, C₁₋₆-alkylen-OH or thiadiazolyl, optionally substituted with C₁₋₆-alkyl;
      R^{1.1.2} H, C₁₋₆-alkyl, SO₂C₁₋₆-alkyl;
      or R^{1.1.1} and R^{1.1.2} together are forming a four-, five- or six-membered carbocyclic ring, optionally containing one N or O, replacing a carbon atom of the ring, optionally substituted with one or two residues selected from the group consisting of C₁₋₆-alkyl, C₁₋₄-alkylene-OH, OH, ═O; or
   R^{1.1} is phenyl, wherein two adjacent residues are together forming a five- or six-membered carbocyclic aromatic or non-aromatic ring, optionally containing independently from each other one or two N, S, or SO₂, replacing a carbon atom of the ring, wherein the ring is optionally substituted with C₁-₄-alkyl or ═O;
   R^{1.2} is selected from
      heteroaryl, optionally substituted with one or two residues selected from the group consisting of C₁₋₆-alkyl, C₂₋₆-alkenyl, C₂₋₆-alkynyl, C₃₋₆-cycloalkyl, CH₂COO-C₁₋₆-alkyl, CONR^{1.2.1}R^{1.2.2}, COR^{1.2.3}, COO-C₁₋₆-alkyl, CONH₂, O-C₁₋₆-alkyl, halogen, CN, SO₂N(C₁₋₄-alkyl)₂ or heteroaryl optionally substituted with one or two residues selected from the group consisting of C₁₋₆-alkyl;
      heteroaryl, optionally substituted with a five- or six-membered carbocyclic non-aromatic ring containing independently from each other two N, O, S, or SO₂, replacing a carbon atom of the ring;
      R^{1.2.1} H, C₁₋₆-alkyl, C₁₋₆-alkylene-C₃₋₆-cycloalkyl, C₁₋₄-alkylene-phenyl, C ₁₋₄-alkylene-furanyl, C₃₋₆-cycloalkyl, C₁₋₄-alkylene-O-C₁₋₄-alkyl, C₁₋₆-haloalkyl or a five- or six-membered carbocyclic non-aromatic ring, optionally containing independently from each other one or two N, O, S, or SO₂, replacing a carbon atom of the ring, optionally substituted with 4-cyclopropylmethyl-piperazinyl
      R^{1.2.2} H, C₁₋₆-alkyl;
      R^{1.2.3} a five- or six-membered carbocyclic non-aromatic ring, optionally containing independently from each other one or two N, O, S, or SO₂, replacing a carbon atom of the ring;
   R^{1.3} is selected from phenyl, heteroaryl or indolyl, each optionally substituted with one or two residues selected from the group consisting of C₁₋₆-alkyl, C₃₋₆-cycloalkyl, O-C₁₋₆-alkyl,O-C₁₋₆-haloalkyl, phenyl, heteroaryl; where in some instances R^{1.3} is selected from phenyl, pyrazolyl, isoxazolyl, pyridinyl, pyrimidinyl, indolyl or oxadiazolyl, each optionally substituted with one or two residues selected from the group consisting of C₁₋₆-alkyl, C₃₋₆-cycloalkyl, O-C₁₋₆-alkyl, O-C₁₋₆-haloalkyl, phenyl, pyrrolidinyl;
R² is selected from the group consisting of C₁₋₆-alkylene-phenyl, C₁₋₆-alkylene-naphthyl, and C₁₋₆-alkylene-thiophenyl; each optionally substituted with one, two or three residues selected from the group consisting of C₁₋₆-alkyl, C₁₋₆-haloalkyl, O-C₁₋₆-alkyl, O-C₁₋₆-haloalkyl, halogen;
R³ is H, C₁₋₄-alkyl;
R⁴ is H, C₁₋₄-alkyl;
   or R³ and R⁴ together are forming a CH₂—CH₂ group.

Another embodiment of the present invention further comprises administration to a subject of the compounds of formula 1 (above), wherein
A is CH₂, O or N-C₁₋₄-alkyl;
R¹ is selected from
   NHR^{1.1}, NMeR^{1.1};
   R^{1.1} is phenyl, optionally substituted with one or two residues selected from the group consisting of C₁₋₆-alkyl, C₂₋₆-alkenyl, C₂₋₆-alkynyl, C₁₋₆-haloalkyl, C₁₋₆-alkylene-OH, C₂₋₆-alkenylene-OH, C₂₋₆-alkynylene-OH, CH₂CON(C₁₋₆-alkyl)₂, CH₂NHCONH-C₃₋₆-cycloalkyl, CN, CO-pyridinyl, CONR^{1.1.1}_{R}^{1.1.2}, COO-C₁₋₆-alkyl, N(SO₂-C₁₋₆-alkyl)(CH₂CON(C₁₋₄-alkyl)₂) O-C₁₋₆-alkyl, O-pyridinyl, SO₂-C₁₋₆-alkyl, SOa-C₁₋₆-alkylen-OH, SO₂-C₃₋₆-cycloalkyl, SO₂-piperidinyl, SO₂NH-C₁₋₆-alkyl, SO₂N(C₁₋₆-alkyl)₂, halogen, CN, CO-morpholinyl, CH₂-pyridinyl or a heterocyclic ring optionally substituted with one or two residues selected from the group consisting of C₁₋₆-alkyl, NHC₁₋₆-alkyl,, =O;
      R^{1.1.1} H, C₁₋₆-alkyl, C₃₋₆-cycloalkyl, C₁₋₆-haloalkyl, CH₂CON(C₁₋₆-alkyl,)₂, CH₂CO-azetindinyl, C₁₋₆-alkylen-C₃₋₆-cycloalkyl, CH₂-pyranyl, CH₂-tetrahydrofuranyl, CH₂-furanyl, C₁₋₆-alkylen-OH or thiadiazolyl, optionally substituted with C₁₋₆-alkyl;
      R^{1.1.2} H, C₁₋₆-alkyl, SO₂C₁₋₆-alkyl;
      or R^{1.1.1} and R^{1.1.2} together are forming a four-, five- or six-membered carbocyclic ring, optionally containing one N or O, replacing a carbon atom of the ring, optionally substituted with one or two residues selected from the group consisting of C₁₋₆-alkyl, C₁₋₄-alkylene-OH, OH, ═O; or
   R^{1.1} is phenyl, wherein two adjacent residues are together forming a five- or six-membered carbocyclic aromatic or non-aromatic ring, optionally containing independently from each other one or two N, S, or SO₂, replacing a carbon atom of the ring, wherein the ring is optionally substituted with C₁-₄-alkyl or ═O;
R² is selected from the group consisting of C₁₋₆-alkylene-phenyl, C₁₋₆-alkylene-naphthyl, and C₁₋₆-alkylene-thiophenyl; each optionally substituted with one, two or three residues selected from the group consisting of C₁₋₆-alkyl, C₁₋₆-haloalkyl, O-C₁₋₆-alkyl, O-C₁₋₆-haloalkyl, halogen;
R³ is H, C₁₋₄-alkyl;
R⁴ is H, C₁₋₄-alkyl;
   or R³ and R⁴ together are forming a CH₂—CH₂ group.

Another embodiment of the present invention further comprises administration to a subject of the compounds of formula 1, wherein
A is CH₂, O or N-C₁₋₄-alkyl;
R¹ is selected from
   NHR^{1.2}, NMeR^{1.2};
   R^{1.2} is selected from
      heteroaryl, optionally substituted with one or two residues selected from the group consisting of C₁₋₆-alkyl, C₂₋₆-alkenyl, C₂₋₆-alkynyl, C₃₋₆-cycloalkyl, CH₂COO-C₁₋₆-alkyl, CONR^{1.2.1}R^{1.2.2}, COR^{1.2.3}, COO-C₁₋₆-alkyl, CONH₂, O-C₁₋₆-alkyl, halogen, CN, SO₂N(C₁₋₄-alkyl)₂ or heteroaryl optionally substituted with one or two residues selected from the group consisting of C₁₋₆-alkyl;
      heteroaryl, optionally substituted with a five- or six-membered carbocyclic non-aromatic ring containing independently from each other two N, O, S, or SO₂, replacing a carbon atom of the ring;
      benzothiazolyl, indazolyl, dihydro-indolyl, indanyl, tetrahydro-quinolinyl, each optionally substituted with one or two residues selected from the group consisting of N(C₁₋₆-alkyl)₂, CONH-C₁₋₆-alkyl, ═O;
      piperidinyl, optionally substituted with pyridinyl;
      4,5-dihydro-naphtho[2,1-d]thiazole, optionally substituted with NHCO-C₁₋₆-alkyl,
      R^{1.2.1} H, C₁₋₆-alkyl, C₁₋₆-alkylene-C₃₋₆-cycloalkyl, C₁₋₄-alkylene-phenyl, C ₁₋₄-alkylene-furanyl, C₃₋₆-cycloalkyl, C₁₋₄-alkylene-O-C₁₋₄-alkyl, C₁₋₆-haloalkyl or a five- or six-membered carbocyclic non-aromatic ring, optionally containing independently from each other one or two N, O, S, or SO₂, replacing a carbon atom of the ring, optionally substituted with 4-cyclopropylmethyl-piperazinyl
      R^{1.2.2} H, C₁₋₆-alkyl;
      R^{1.2.3} a five- or six-membered carbocyclic non-aromatic ring, optionally containing independently from each other one or two N, O, S, or SO₂, replacing a carbon atom of the ring;
R² is selected from the group consisting of C₁₋₆-alkylene-phenyl, C₁₋₆-alkylene-naphthyl, and C₁₋₆-alkylene-thiophenyl; each optionally substituted with one, two or three residues selected from the group consisting of C₁₋₆-alkyl, C₁₋₆-haloalkyl, O-C₁₋₆-alkyl, O-C₁₋₆-haloalkyl, halogen;
R³ is H, C₁₋₄-alkyl;
R⁴ is H, C₁₋₄-alkyl;
   or R³ and R⁴ together are forming a CH₂—CH₂ group.

Another embodiment of the present invention further comprises administration to a subject of the compounds of formula 1 (above), wherein
A is CH₂, O or N-C₁₋₄-alkyl;
R¹ is selected from
   NHR^{1.2}, NMeR^{1.2};
   R^{1.2} is selected from
      heteroaryl, optionally substituted with one or two residues selected from the group consisting of C₁₋₆-alkyl, C₂₋₆-alkenyl, C₂₋₆-alkynyl, C₃₋₆-cycloalkyl, CH₂COO-C₁₋₆-alkyl, CONR^{1.2.1}R^{1.2.2}, COR^{1.2.3}, COO-C₁₋₆-alkyl, CONH₂, O-C₁₋₆-alkyl, halogen, CN, SO₂N(C₁₋₄-alkyl)₂ or heteroaryl optionally substituted with one or two residues selected from the group consisting of C₁₋₆-alkyl;
      heteroaryl, optionally substituted with a five- or six-membered carbocyclic non-aromatic ring containing independently from each other two N, O, S, or SO₂, replacing a carbon atom of the ring;
      R^{1.2.1} H, C₁₋₆-alkyl, C₁₋₆-alkylene-C₃₋₆-cycloalkyl, C₁₋₄-alkylene-phenyl, C ₁₋₄-alkylene-furanyl, C₃₋₆-cycloalkyl, C₁₋₄-alkylene-O-C₁₋₄-alkyl, C₁₋₆-haloalkyl or a five- or six-membered carbocyclic non-aromatic ring, optionally containing independently from each other one or two N, O, S, or SO₂, replacing a carbon atom of the ring, optionally substituted with 4-cyclopropylmethyl-piperazinyl
      R^{1.2.2} H, C₁₋₆-alkyl;
      R^{1.2.3} a five- or six-membered carbocyclic non-aromatic ring, optionally containing independently from each other one or two N, O, S, or SO₂, replacing a carbon atom of the ring;
R² is selected from the group consisting of C₁₋₆-alkylene-phenyl, C₁₋₆-alkylene-naphthyl, and C₁₋₆-alkylene-thiophenyl; each optionally substituted with one, two or three residues selected from the group consisting of C₁₋₆-alkyl, C₁₋₆-haloalkyl, O-C₁₋₆-alkyl, O-C₁₋₆-haloalkyl, halogen;
R³ is H, C₁₋₄-alkyl;
R⁴ is H, C₁₋₄-alkyl;
   or R³ and R⁴ together are forming a CH₂—CH₂ group.

Another embodiment of the present invention further comprises administration to a subject of the compounds of formula 1, wherein
A is CH₂, O or N-C₁₋₄-alkyl;
R¹ is selected from
   NHCH₂-R^{1.3};
   R^{1.3} is selected from phenyl, pyrazolyl, isoxazolyl, pyridinyl, pyrimidinyl, indolyl or oxadiazolyl, each optionally substituted with one or two residues selected from the group consisting of C₁₋₆-alkyl, C₃₋₆-cycloalkyl, O-C₁₋₆-alkyl, O-C₁₋₆-haloalkyl, phenyl, pyrrolidinyl;
R² is selected from the group consisting of C₁₋₆-alkylene-phenyl, C₁₋₆-alkylene-naphthyl, and C₁₋₆-alkylene-thiophenyl; each optionally substituted with one, two or three residues selected from the group consisting of C₁₋₆-alkyl, C₁₋₆-haloalkyl, O-C₁₋₆-alkyl, O-C₁₋₆-haloalkyl, halogen;
R³ is H, C₁₋₄-alkyl;
R⁴ is H, C₁₋₄-alkyl;
   or R³ and R⁴ together are forming a CH₂—CH₂ group.

Another embodiment of the present invention further comprises administration to a subject of the compounds of formula 1, wherein
A is CH₂, O or N-C₁₋₄-alkyl;
R¹ is selected from
   NHR^{1.1}, NMeR^{1.1};
   NHR^{1.2}, NMeR^{1.2};
   NHCH₂-R^{1.3};
   NH-C₃₋₆-cycloalkyl, whereas optionally one carbon atom is replaced by a nitrogen atom, whereas the ring is optionally substituted with one or two residues selected from the group consisting of C₁₋₆-alkyl, O-C₁₋₆-alkyl, NHSO₂-phenyl, NHCONH-phenyl, halogen, CN, SO₂-C₁₋₆-alkyl, COO-C₁₋₆-alkyl;
   a C₉ ₒᵣ ₁₀-bicyclic-ring, whereas one or two carbon atoms are replaced by nitrogen atoms and the ring system is bound via a nitrogen atom to the basic structure of formula 1 and whereas the ring system is optionally substituted with one or two residues selected from the group consisting of C₁₋₆-alkyl, COO-C₁₋₆-alkyl, C₁₋₆-haloalkyl, O-C₁₋₆-alkyl, NO₂, halogen, CN, NHSO₂-C₁₋₆-alkyl, m-methoxyphenyl;
   a group selected from NHCH(pyridinyl)CH₂COO-C₁₋₆-alkyl, NHCH(CH₂O-C₁₋₆-alkyl)-benzoimidazolyl, optionally substituted with C1;
   or 1-aminocyclopentyl, optionally substituted with methyl-oxadiazolyl;
   R^{1.1} is phenyl, optionally substituted with one or two residues selected from the group consisting of C₁₋₆-alkyl, C₁₋₆-haloalkyl, CH₂CON(C₁₋₆-alkyl)₂, CH₂NHCONH-C₃₋₆-cycloalkyl, CN, CONR^{1.1.1}_{R}^{1.1.2},COO-C₁₋₆-alkyl, O-C₁₋₆-alkyl, SO₂-C₁₋₆-alkyl, SOa-C₁₋₆-alkylen-OH, SO₂-C₃₋₆-cycloalkyl, SO₂-piperidinyl, SO₂NH-C₁₋₆-alkyl, SO₂N(C₁₋₆-alkyl)₂, halogen, CN, CO-morpholinyl, CH₂-pyridinyl or a heterocyclic ring optionally substituted with one or two residues selected from the group consisting of C₁₋₆-alkyl, NHC₁₋₆-alkyl,, =O;
      R^{1.1.1} H, C₁₋₆-alkyl, C₃₋₆-cycloalkyl, C₁₋₆-haloalkyl, CH₂CON(C₁₋₆-alkyl,)₂, CH₂CO-azetindinyl, C₁₋₆-alkylen-C₃₋₆-cycloalkyl, CH₂-pyranyl, CH₂-tetrahydrofuranyl, CH₂-furanyl, C₁₋₆-alkylen-OH or thiadiazolyl, optionally substituted with C₁₋₆-alkyl;
      R^{1.1.2} H, C₁₋₆-alkyl, SO₂C₁₋₆-alkyl;
      or R^{1.1.1} and R^{1.1.2} together are forming a four-, five- or six-membered carbocyclic ring, optionally containing one O, replacing a carbon atom of the ring, optionally substituted with one or two residues selected from the group consisting of CH₂OH
   R^{1.2} is selected from
      heteroaryl, optionally substituted with one or two residues selected from the group consisting of C₁₋₆-alkyl, C₃₋₆-cycloalkyl, CH₂COO-C₁₋₆-alkyl, CONR^{1.2.1}R^{1.2.2}, COO-C₁₋₆-alkyl, CONH₂, O-C₁₋₆-alkyl, halogen, CN, CO-pyrrolidinyl, CO-morpholinyl or heteroaryl optionally substituted with one or two residues selected from the group consisting of C₁₋₆-alkyl;
      benzothiazolyl, indazolyl, dihydro-indolyl, indanyl, tetrahydro-quinolinyl, each optionally substituted with one or two residues selected from the group consisting of N(C₁₋₆-alkyl)₂, CONH-C₁₋₆-alkyl, ═O;
      piperidinyl, optionally substituted with pyridinyl;
      4,5-dihydro-naphtho[2,1-d]thiazole, optionally substituted with NHCO-C₁₋₆-alkyl,
      R^{1.2.1} H, C₁₋₆-alkyl;
      R^{1.2.2} H, C₁₋₆-alkyl;
   R^{1.3} is selected from phenyl, pyrazolyl, isoxazolyl, pyrimidinyl, indolyl or oxadiazolyl, each optionally substituted with one or two residues selected from the group consisting of C₁₋₆-alkyl, C₃₋₆-cycloalkyl, O-C₁₋₆-alkyl, O-C₁₋₆-haloalkyl;
R² is selected from C₁₋₆-alkylene-phenyl or C₁₋₆-alkylene-naphthyl, both optionally substituted with one or two residues selected from the group consisting of C₁₋₆-alkyl, C₁₋₆-haloalkyl, O-C₁₋₆-alkyl, O-C₁₋₆-haloalkyl, halogen; or CH₂-thiophenyl, optionally substituted with one or two residues selected from the group consisting of halogen;
R³ is H, C₁₋₄-alkyl;
R⁴ is H, C₁₋₄-alkyl;
   or R³ and R⁴ together are forming a CH₂—CH₂ group.

Another embodiment of the present invention further comprises administration to a subject of the compounds of formula 1, wherein
A is CH₂, O or NMe;
R¹ is selected from
   NHR^{1.1}, NMeR^{1.1};
   NHR^{1.2}, NMeR^{1.2;}
   NHCH₂-R^{1.3};
   NH-cyclohexyl, optionally substituted with one or two residues selected from the group consisting of C₁₋₄-alkyl, NHSO₂-phenyl, NHCONH-phenyl, halogen;
   NH-pyrrolidinyl, optionally substituted with one or two residues selected from the group consisting of SO₂-C₁₋₄-alkyl, COO-C₁₋₄-alkyl;
   piperidinyl, optionally substituted with one or two residues selected from the group consisting of NHSO₂-C₁₋₄-alkyl, m-methoxyphenyl;
   dihydro-indolyl, dihydro-isoindolyl, tetrahydro-quinolinyl or tetrahydro-isoquinolinyl, optionally substituted with one or two residues selected from the group consisting of C₁₋₄-alkyl, COO-C₁₋₄-alkyl, C₁₋₄-haloalkyl, O-C₁₋₄-alkyl, NO₂, halogen;
   a group selected from NHCH(pyridinyl)CH₂COO-C₁₋₄-alkyl, NHCH(CH₂O-C₁₋₄-alkyl)-benzoimidazolyl, optionally substituted with Cl;
   or 1-aminocyclopentyl, optionally substituted with methyl-oxadiazolyl;
   R^{1.1} is phenyl, optionally substituted with one or two residues selected from the group consisting of C₁₋₄-alkyl, C₁₋₄-haloalkyl, CH₂CON(C₁₋₄-alkyl)₂, CH₂NHCONH-C₃₋₆-cycloalkyl, CN, CONR^{1.1.1}R^{1.1.2}, COO-C₁₋₄-alkyl, O-C₁₋₄-alkyl, SO₂-C₁₋₄-alkyl, SO₂-C₁₋₄-alkylen-OH, SO₂-C₃₋₆-cycloalkyl, SO₂-piperidinyl, SO₂NH-C₁₋₄-alkyl, SO₂N(C₁₋₄-alkyl)₂, halogen, CO-morpholinyl, CH₂-pyridinyl, or imidazolidinyl, piperidinyl, oxazinanyl, pyrazolyl, triazolyl, tetrazolyl, oxazolyl, oxadiazolyl, thiazolyl, pyridinyl, pyrimidinyl, each optionally substituted with one or two residues selected from the group consisting of C₁₋₄-alkyl, NHC₁₋₄-alkyl,, =O;
      R^{1.1.1} H, C₁₋₆-alkyl, C₃₋₆-cycloalkyl, C₁₋₄-haloalkyl, CH₂CON(C₁₋₄-alkyl,)₂, CH₂CO-azetindinyl, C₁₋₄-alkylen-C₃₋₆-cycloalkyl, CH₂-pyranyl, CH₂-tetrahydrofuranyl, CH₂-furanyl, C₁₋₄-alkylen-OH or thiadiazolyl, optionally substituted with C₁₋₄-alkyl;
      R^{1.1.2} H, C₁₋₄-alkyl, SO₂C₁₋₄-alkyl;
      or R^{1.1.1} and R^{1.1.2} together are forming a four-, five- or six-membered carbocyclic ring, optionally containing one O, replacing a carbon atom of the ring, optionally substituted with one or two residues selected from the group consisting of CH₂OH
   R^{1.2} is selected from
      pyridinyl, pyridazinyl, pyrrolyl, pyrazolyl, isoxazolyl, thiazolyl, thiadiazolyl, optionally substituted with one or two residues selected from the group consisting of C₁₋₄-alkyl, C₃₋₆-cycloalkyl, CH₂COO-C₁₋₄-alkyl, CONR^{1.2.1}R^{1.2.2}, COO-C₁₋₄-alkyl, CONH₂, O-C₁₋₄-alkyl, halogen, CO-pyrrolidinyl, CO-morpholinyl or pyrazolyl, triazolyl, tetrazolyl, isoxazolyl, oxadiazolyl, each optionally substituted with one or two residues selected from the group consisting of C₁₋₄-alkyl;
      benzothiazolyl, indazolyl, dihydro-indolyl, indanyl, tetrahydro-quinolinyl, each optionally substituted with one or two residues selected from the group consisting of N(C₁₋₄-alkyl)₂, CONH-C₁₋₄-alkyl, =O;
      piperidinyl, optionally substituted with pyridinyl;
      4,5-dihydro-naphtho[2,1-d]thiazole, optionally substituted with NHCO-C₁₋₄-alkyl,
      R^{1.2.1} H, C₁₋₄-alkyl;
      R^{1.2.2} H, C₁₋₄-alkyl;
   R^{1.3} is selected from phenyl, pyrazolyl, isoxazolyl, pyrimidinyl, indolyl or oxadiazolyl, each optionally substituted with one or two residues selected from the group consisting of C₁₋₄-alkyl, C₃₋₆-cycloalkyl, O-C₁₋₄-alkyl, O-C₁₋₄-haloalkyl;
R² is selected from C₁₋₆-alkylene-phenyl or C₁₋₆-alkylene-naphthyl, both optionally substituted with one or two residues selected from the group consisting of C₁₋₄-alkyl, C₁₋₄-haloalkyl, O-C₁₋₄-haloalkyl, halogen; or CH₂-thiophenyl, optionally substituted with one or two residues selected from the group consisting of halogen;
R³ is H;
R⁴ is H;
   or R³ and R⁴ together are forming a CH₂—CH₂ group.

Another embodiment of the present invention further comprises administration to a subject of the compounds of formula 1, wherein
A is CH₂, O or NMe;
R¹ is selected from
   NHR^{1.1}, NMeR^{1.1};
   NHR^{1.2}, NMeR^{1.2;}
   NHCH₂-R^{1.3};
   NH-piperidinyl, optionally substituted with pyridinyl;
   NH-cyclohexyl, optionally substituted with one or two residues selected from the group consisting of t-Bu, NHSO₂-phenyl, NHCONH-phenyl, F;
   NH-pyrrolidinyl, optionally substituted with one or two residues selected from the group consisting of SO₂Me, COO-t-Bu;
   piperidinyl, optionally substituted with one or two residues selected from the group consisting of NHSO₂-n-Bu, m-methoxyphenyl;
   dihydro-indolyl, dihydro-isoindolyl, tetrahydro-quinolinyl or tetrahydro-isoquinolinyl, optionally substituted with one or two residues selected from the group consisting of Me, COOMe, CF₃, OMe, NO₂, F, Br;
   a group selected from NHCH(pyridinyl)CH₂COOMe, NHCH(CH₂OMe)-benzoimidazolyl, optionally substituted with Cl;
   or 1-aminocyclopentyl, optionally substituted with methyl-oxadiazolyl;
   R^{1.1} is phenyl, optionally substituted with one or two residues selected from the group consisting of Me, Et, t-Bu, CF₃, CH₂CONMe₂, CH₂NHCONH-cyclohexyl, CN, CONR^{1.1.1}R^{1.1.2}, COOMe, COOEt, OMe, SO₂Me, SO₂CH₂CH₂OH, SO₂Et, SO₂-cyclopropyl, SO₂-piperidinyl, SO₂NHEt, SO₂NMeEt, F, Cl, CO-morpholinyl, CH₂-pyridinyl, or imidazolidinyl, piperidinyl, oxazinanyl, pyrazolyl, triazolyl, tetrazolyl, oxazolyl, oxadiazolyl, thiazolyl, pyridinyl, pyrimidinyl, each optionally substituted with one or two residues selected from the group consisting of Me, NHMe, =O;
      R^{1.1.1} H, Me, Et, t-Bu, i-Pr, cyclopropyl, CH₂-i-Pr, CH₂-t-Bu, CH(CH₃)CH₂CH₃, CH₂CHF₂, CH₂CONMe₂, CH₂CO-azetindinyl, CH₂-cyclopropyl, CH₂-cyclobutyl, CH₂-pyranyl, CH₂-tetrahydrofuranyl, CH₂-furanyl, CH₂CH₂OH or thiadiazolyl, optionally substituted with Me;
      R^{1.1.2} H, Me, Et, SO₂Me, SO₂Et or R^{1.1.1} and R^{1.1.2} together are forming a four-, five- or six-membered carbocyclic ring, optionally containing one O, replacing a carbon atom of the ring, optionally substituted with one or two residues selected from the group consisting of CH₂OH
   R^{1.2} is selected from
      pyridinyl, pyrrolyl, pyrazolyl, isoxazolyl, thiazolyl, thiadiazolyl, optionally substituted with one or two residues selected from the group consisting of Me, Et, Pr, Bu, cyclopropyl, CH₂COOEt, CONR^{1.2.1}R^{1.2.2}, COOMe, COOEt, CONH₂, OMe, Cl, Br CO-pyrrolidinyl, CO-morpholinyl or pyrazolyl, triazolyl, tetrazolyl, isoxazolyl, oxadiazolyl, each optionally substituted Me;
      benzothiazolyl, indazolyl, dihydro-indolyl, indanyl, tetrahydro-quinolinyl, each optionally substituted with one or two residues selected from the group consisting of NMe₂, CONHMe, ═O;
      4,5-dihydro-naphtho[2,1-d]thiazole, optionally substituted with NHCOMe,
      R^{1.2.1} H, Me;
      R^{1.2.2} H, Me;
   R^{1.3} is selected from phenyl, pyrazolyl, isoxazolyl, pyrimidinyl, indolyl or oxadiazolyl, each optionally substituted with one or two residues selected from the group consisting of Me, Et, Pr, cyclopentyl, OMe, OCHF₂;
R² is selected from CH₂-phenyl or CH₂-naphthyl, both optionally substituted with one or two residues selected from the group consisting of CH₃, CF₃, OCF₃, F, Cl, Br, Et; or CH₂-thiophenyl, optionally substituted with one or two residues selected from the group consisting of Cl, Br;
R³ is H;
R⁴ is H;
   or R³ and R⁴ together are forming a CH₂—CH₂ group.

Another embodiment of the present invention further comprises administration to a subject of the compounds of formula 1, wherein
A is CH₂, O or NMe;
R¹ is selected from
   NHR^{1.1}
   NHR^{1.2},
   R^{1.1} is phenyl, optionally substituted with one or two residues selected from the group consisting of Me, Et, Pr, Bu, CF₃, CH₂CONMe₂, CH₂NHCONH-cyclohexyl, CN, CONR^{1.1.1}R^{1.1.2}, COOMe, COOEt, OMe, SO₂Me, SO₂CH₂CH₂OH, SO₂Et, SO₂-cyclopropyl, SO₂-piperidinyl, SO₂NHEt, SO₂NMeEt, F, Cl, CO-morpholinyl, CH₂-pyridinyl, or imidazolidinyl, piperidinyl, oxazinanyl, pyrazolyl, triazolyl, tetrazolyl, oxazolyl, oxadiazolyl, thiazolyl, pyridinyl, pyrimidinyl, each optionally substituted with one or two residues selected from the group consisting of Me, NHMe, ═O;
      R^{1.1.1} H, Me, Et, t-Bu, i-Pr, cyclopropyl, CH₂-i-Pr, CH₂-t-Bu, CH(CH₃)CH₂CH₃, CH₂CHF₂, CH₂CONMe₂, CH₂CO-azetindinyl, CH₂-cyclopropyl, CH₂-cyclobutyl, CH₂-pyranyl, CH₂-tetrahydrofuranyl, CH₂-furanyl, CH₂CH₂OH or thiadiazolyl, optionally substituted with Me;
      R^{1.1.2} H, Me, Et, SO₂Me, SO₂Et
      or R^{1.1.1} and R^{1.1.2} together are forming a four-, five- or six-membered carbocyclic ring, optionally containing one O, replacing a carbon atom of the ring, optionally substituted with one or two residues selected from the group consisting of CH₂OH
   R^{1.2} is selected from
      pyridinyl, pyrrolyl, pyrazolyl, isoxazolyl, thiazolyl, thiadiazolyl, optionally substituted with one or two residues selected from the group consisting of Me, Et, Pr, Bu, cyclopropyl, CH₂COOEt, CONR^{1.2.1}R^{1.2.2}, COOMe, COOEt, CONH₂, OMe, Cl, Br CO-pyrrolidinyl, CO-morpholinyl or pyrazolyl, triazolyl, tetrazolyl, isoxazolyl, oxadiazolyl, each optionally substituted Me;
      benzothiazolyl, indazolyl, dihydro-indolyl, indanyl, tetrahydro-quinolinyl, each optionally substituted with one or two residues selected from the group consisting of NMe₂, CONHMe, =O;
      4,5-dihydro-naphtho[2,1-d]thiazole, optionally substituted with NHCOMe,
      R^{1.2.1} H, Me;
      R^{1.2.2} H, Me;
R² is selected from CH₂-phenyl or CH₂-naphthyl, both optionally substituted with one or two residues selected from the group consisting of CH₃, CF₃, OCF₃, F, Cl, Br, Et
R³ is H;
R⁴ is H.

Another embodiment of the present invention further comprises administration to a subject of the compounds of formula 1, wherein
A is CH₂, O or NMe;
R¹ is selected from
   NHR^{1.1}, NMeR^{1.1};
   NHR^{1.2}, NMeR^{1.2};
   NHCH₂-R^{1.3};
   R¹¹ is phenyl, optionally substituted with one or two residues selected from the group consisting of Me, Et, Pr, Bu, CF₃, CH₂CONMe₂, CH₂NHCONH-cyclohexyl, CN, CONR^{1.1.1}R^{1.1.2}, COOMe, COOEt, OMe, SO₂Me, SO₂CH₂CH₂OH, SO₂Et, SO₂-cyclopropyl, SO₂-piperidinyl, SO₂NHEt, SO₂NMeEt, F, Cl, CO-morpholinyl, CH₂-pyridinyl, or imidazolidinyl, piperidinyl, oxazinanyl, pyrazolyl, triazolyl, tetrazolyl, oxazolyl, oxadiazolyl, thiazolyl, pyridinyl, pyrimidinyl, each optionally substituted with one or two residues selected from the group consisting of Me, NHMe, ═O;
   R^{1.1.1} H, Me, Et, Pr, Bu, cyclopropyl, CH₂—Pr, CH₂—Bu, CH(CH₃)CH₂CH₃, CH₂CHF₂, CH₂CONMe₂, CH₂CO-azetindinyl, CH₂-cyclopropyl, CH₂-cyclobutyl, CH₂-pyranyl, CH₂-tetrahydrofuranyl, CH₂-furanyl, CH₂CH₂OH or thiadiazolyl, optionally substituted with Me;
   R^{1.1.2} H, Me, Et, SO₂Me, SO₂Et or R^{1.1.1} and R^{1.1.2} together are forming a four-, five- or six-membered carbocyclic ring, optionally containing one O, replacing a carbon atom of the ring, optionally substituted with one or two residues selected from the group consisting of CH₂OH
   R^{1.2} is selected from
      pyridinyl, pyrrolyl, pyrazolyl, isoxazolyl, thiazolyl, thiadiazolyl, optionally substituted with one or two residues selected from the group consisting of Me, Et, Pr, Bu, cyclopropyl, CH₂COOEt, CONR^{1.2.1}R^{1.2.2}, COOMe, COOEt, CONH₂, OMe, Cl, Br CO-pyrrolidinyl, CO-morpholinyl or pyrazolyl, triazolyl, tetrazolyl, isoxazolyl, oxadiazolyl, each optionally substituted Me;
      benzothiazolyl, indazolyl, dihydro-indolyl, indanyl, tetrahydro-quinolinyl, each optionally substituted with one or two residues selected from the group consisting of NMe₂, CONHMe, =O;
      4,5-dihydro-naphtho[2,1-d]thiazole, optionally substituted with NHCOMe,
      R^{1.2.1} H, Me;
      R^{1.2.2} H, Me;
   R^{1.3} is selected from phenyl, pyrazolyl, isoxazolyl, pyrimidinyl, indolyl or oxadiazolyl, each optionally substituted with one or two residues selected from the group consisting of Me, Et, Pr, cyclopentyl, OMe, OCHF₂;
R² is selected from CH₂-phenyl or CH₂-naphthyl, both optionally substituted with one or two residues selected from the group consisting of CH₃, CF₃, OCF₃, F, Cl, Br, Et; or CH₂-thiophenyl, optionally substituted with one or two residues selected from the group consisting of Cl, Br;
R³ is H;
R⁴ is H;
   or R³ and R⁴ together are forming a CH₂—CH₂ group.

Another embodiment of the present invention further comprises administration to a subject of the compounds of formula 1, wherein
A is CH₂, O or NMe;
R¹ is selected from
   NHR^{1.1}, NMeR^{1.1};
   R^{1.1} is phenyl, optionally substituted with one or two residues selected from the group consisting of Me, Et, t-Bu, CF₃, CH₂CONMe₂, CH₂NHCONH-cyclohexyl, CN, CONR^{1.1.1}R^{1.1.2}, COOMe, COOEt, OMe, SO₂Me, SO₂CH₂CH₂OH, SO₂Et, SO₂-cyclopropyl, SO₂-piperidinyl, SO₂NHEt, SO₂NMeEt, F, Cl, CO-morpholinyl, CH₂-pyridinyl, or imidazolidinyl, piperidinyl, oxazinanyl, pyrazolyl, triazolyl, tetrazolyl, oxazolyl, oxadiazolyl, thiazolyl, pyridinyl, pyrimidinyl, each optionally substituted with one or two residues selected from the group consisting of Me, NHMe, ═O;
      R^{1.1.1} H, Me, Et, Bu, Pr, cyclopropyl, CH₂—Pr, CH₂—Bu, CH(CH₃)CH₂CH₃, CH₂CHF₂, CH₂CONMe₂, CH₂CO-azetindinyl, CH₂-cyclopropyl, CH₂-cyclobutyl, CH₂-pyranyl, CH₂-tetrahydrofuranyl, CH₂-furanyl, CH₂CH₂OH or thiadiazolyl, optionally substituted with Me;
      R^{1.1.2} H, Me, Et, SO₂Me, SO₂Et or R^{1.1.1} and R^{1.1.2} together are forming a four-, five- or six-membered carbocyclic ring, optionally containing one O, replacing a carbon atom of the ring, optionally substituted with one or two residues selected from the group consisting of CH₂OH;
R² is selected from CH₂-phenyl or CH₂-naphthyl, both optionally substituted with one or two residues selected from the group consisting of CH₃, CF₃, OCF₃, F, Cl, Br, Et; or CH₂-thiophenyl, optionally substituted with one or two residues selected from the group consisting of Cl, Br;
R³ is H;
R⁴ is H;
   or R³ and R⁴ together are forming a CH₂—CH₂ group.

Another embodiment of the present invention further comprises administration to a subject of the compounds of formula 1, wherein
A is CH₂, O or NMe;
R¹ is selected from
   NHR^{1.1}, NMeR^{1.1};
   R^{1.1} is phenyl, optionally substituted with one or two residues selected from the group consisting of Me, Et, t-Bu, CF₃, CH₂CONMe₂, CH₂NHCONH-cyclohexyl, CN, CONR^{1.1.1}R^{1.1.2}, COOMe, COOEt, OMe, SO₂Me, SO₂CH₂CH₂OH, SO₂Et, SO₂-cyclopropyl, SO₂-piperidinyl, SO₂NHEt, SO₂NMeEt, F, Cl, CO-morpholinyl, CH₂-pyridinyl, or imidazolidinyl, piperidinyl, oxazinanyl, pyrazolyl, triazolyl, tetrazolyl, oxazolyl, oxadiazolyl, thiazolyl, pyridinyl, pyrimidinyl, each optionally substituted with one or two residues selected from the group consisting of Me, NHMe, =O;
      R^{1.1.1} H, Me, Et, Bu, Pr, cyclopropyl, CH₂—Pr, CH₂—Bu, CH(CH₃)CH₂CH₃, CH₂CHF₂, CH₂CONMe₂, CH₂CO-azetindinyl, CH₂-cyclopropyl, CH₂-cyclobutyl, CH₂-pyranyl, CH₂-tetrahydrofuranyl, CH₂-furanyl, CH₂CH₂OH or thiadiazolyl, optionally substituted with Me;
      R^{1.1.2} H, Me, Et, SO₂Me, SO₂Et
      or R^{1.1.1} and R^{1.1.2} together are forming a four-, five- or six-membered carbocyclic ring, optionally containing one O, replacing a carbon atom of the ring, optionally substituted with one or two residues selected from the group consisting of CH₂OH;
R² is defined as in Table 1 shown below;
R³ is H;
R⁴ is H;

Another embodiment of the present invention further comprises administration to a subject of the compounds of formula 1, wherein
A is CH₂, O or NMe;
R¹ is selected from
   NHR^{1.1}, NMeR^{1.1};
   R^{1.1} is phenyl, optionally substituted with one or two residues selected from the group consisting of Me, Et, t-Bu, CF₃, CH₂CONMe₂, CH₂NHCONH-cyclohexyl, CN, CONR^{1.1.1}R^{1.1.2}, COOMe, COOEt, OMe, SO₂Me, SO₂CH₂CH₂OH, SO₂Et, SO₂-cyclopropyl, SO₂-piperidinyl, SO₂NHEt, SO₂NMeEt, F, Cl, CO-morpholinyl, CH₂-pyridinyl, or imidazolidinyl, piperidinyl, oxazinanyl, pyrazolyl, triazolyl, tetrazolyl, oxazolyl, oxadiazolyl, thiazolyl, pyridinyl, pyrimidinyl, each optionally substituted with one or two residues selected from the group consisting of Me, NHMe, =O;
      and R^{1.1.1} and R^{1.1.2} together are forming a four-, five- or six-membered carbocyclic ring, optionally containing one O, replacing a carbon atom of the ring, optionally substituted with one or two residues selected from the group consisting of CH₂OH;
R² is defined as in Table 1 shown below;
R³ is H;
R⁴ is H;

Another embodiment of the present invention further comprises administration to a subject of the compounds of formula 1, wherein
A is CH₂, O or NMe;
R¹ is selected from
   NHR^{1.1}, NMeR^{1.1};
   R^{1.1} is phenyl, optionally substituted with one or two residues selected from the group consisting of Me, Et, t-Bu, CF₃, CH₂CONMe₂, CH₂NHCONH-cyclohexyl, CN, CONR^{1.1.1}R^{1.1.2}, COOMe, COOEt, OMe, F, Cl;
      R^{1.1.1} H, Me, Et, Bu, Pr, cyclopropyl, CH₂-Pr, CH₂-Bu, CH(CH₃)CH₂CH₃, CH₂CHF₂, CH₂CONMe₂, CH₂CO-azetindinyl, CH₂-cyclopropyl, CH₂-cyclobutyl, CH₂-pyranyl, CH₂-tetrahydrofuranyl, CH₂-furanyl, CH₂CH₂OH or thiadiazolyl, optionally substituted with Me;
      R^{1.1.2} H, Me, Et, SO₂Me, SO₂Et
R² is defined as in Table 1 shown below;
R³ is H;
R⁴ is H;

Another embodiment of the present invention further comprises administration to a subject of the compounds of formula 1, wherein
A is CH₂, O or NMe;
R¹ is selected from
   NHR^{1.1}, NMeR^{1.1};
   R^{1.1} is phenyl, optionally substituted with one or two residues selected from the group consisting of SO₂Me, SO₂CH₂CH₂OH, SO₂Et, SO₂-cyclopropyl, SO₂-piperidinyl, SO₂NHEt, SO₂NMeEt;
      R^{1.1.1} H, Me, Et, Bu, Pr, cyclopropyl, CH₂-Pr, CH₂-Bu, CH(CH₃)CH₂CH₃, CH₂CHF₂, CH₂CONMe₂, CH₂CO-azetindinyl, CH₂-cyclopropyl, CH₂-cyclobutyl, CH₂-pyranyl, CH₂-tetrahydrofuranyl, CH₂-furanyl, CH₂CH₂OH or thiadiazolyl, optionally substituted with Me;
      R^{1.1.2} H, Me, Et, SO₂Me, SO₂Et
R² is defined as in Table 1 shown below;
R³ is H;
R⁴ is H;

Another embodiment of the present invention further comprises administration to a subject of the compounds of formula 1, wherein
A is CH₂, O or NMe;
R¹ is selected from
   NHR^{1.1}, NMeR^{1.1};
   R^{1.1} is phenyl, optionally substituted with one residue selected from the group consisting of Me, Et, t-Bu, CF₃, CH₂CONMe₂, CH₂NHCONH-cyclohexyl, CN, CONR^{1.1.1}R^{1.1.2}, COOMe, COOEt, OMe, SO₂Me, SO₂CH₂CH₂OH, SO₂Et, SO₂-cyclopropyl, SO₂-piperidinyl, SO₂NHEt, SO₂NMeEt, F, Cl, and additionally with one residue selected from the group consiting of CO-morpholinyl, CH₂-pyridinyl, or imidazolidinyl, piperidinyl, oxazinanyl, pyrazolyl, triazolyl, tetrazolyl, oxazolyl, oxadiazolyl, thiazolyl, pyridinyl, pyrimidinyl, each optionally substituted with one or two residues selected from the group consisting of Me, NHMe, =O;
   R^{1.1.1} H, Me, Et, Bu, Pr, cyclopropyl, CH₂—Pr, CH₂—Bu, CH(CH₃)CH₂CH₃, CH₂CHF₂, CH₂CONMe₂, CH₂CO-azetindinyl, CH₂-cyclopropyl, CH₂-cyclobutyl, CH₂-pyranyl, CH₂-tetrahydrofuranyl, CH₂-furanyl, CH₂CH₂OH or thiadiazolyl, optionally substituted with Me;
   R^{1.1.2} H, Me, Et, SO₂Me, SO₂Et
R² is defined as in Table 1 shown below;
R³ is H;
R⁴ is H;

Another embodiment of the present invention further comprises administration to a subject of the compounds of formula 1, wherein
A is CH₂, O or NMe;
R¹ is selected from
   NHR^{1.2}, NMeR^{1.2};
   R ^{1.2} is selected from
      pyridinyl, pyridazinyl, pyrrolyl, pyrazolyl, isoxazolyl, thiazolyl, thiadiazolyl, optionally substituted with one or two residues selected from the group consisting of Me, Et, Pr, Bu, cyclopropyl, CH₂COOEt, CONR^{1.2.1}R^{1.2.2}, COOMe, COOEt, CONH₂, OMe, Cl, Br CO-pyrrolidinyl, CO-morpholinyl or pyrazolyl, triazolyl, tetrazolyl, isoxazolyl, oxadiazolyl, each optionally substituted Me;
      benzothiazolyl, indazolyl, dihydro-indolyl, indanyl, tetrahydro-quinolinyl, each optionally substituted with one or two residues selected from the group consisting of NMe₂, CONHMe, =O;
      4,5-dihydro-naphtho[2,1-d]thiazole, optionally substituted with NHCOMe,
      R^{1.2.1} H, Me;
      R^{1.2.2} H, Me;
R² is selected from CH₂-phenyl or CH₂-naphthyl, both optionally substituted with one or two residues selected from the group consisting of CH₃, CF₃, OCF₃, F, Cl, Br, Et; or CH₂-thiophenyl, optionally substituted with one or two residues selected from the group consisting of Cl, Br;
R³ is H;
R⁴ is H;
   or R³ and R⁴ together are forming a CH₂—CH₂ group.

Another embodiment of the present invention further comprises administration to a subject of the compounds of formula 1, wherein
A is CH₂, O or NMe;
R¹ is selected from
   NHR^{1.2}, NMeR^{1.2};
   R^{1.2} is selected from pyridinyl, pyridazinyl, pyrrolyl, pyrazolyl, isoxazolyl, thiazolyl, thiadiazolyl, optionally substituted with one or two residues selected from the group consisting of Me, Et, n-Pr, i-Pr, Bu, cyclopropyl, CH₂COOEt, CONR^{1.2.1}R^{1.2.2}, COOMe, COOEt, CONH₂, OMe, Cl, Br CO-pyrrolidinyl, CO-morpholinyl or pyrazolyl, triazolyl, tetrazolyl, isoxazolyl, oxadiazolyl, each optionally substituted Me;
      R^{1.2.1} H, Me;
      R^{1.2.2} H, Me;
R² is selected from CH₂-phenyl or CH₂-naphthyl, both optionally substituted with one or two residues selected from the group consisting of CH₃, CF₃, OCF₃, F, Cl, Br, Et; or CH₂-thiophenyl, optionally substituted with one or two residues selected from the group consisting of Cl, Br;
R³ is H;
R⁴ is H;
   or R³ and R⁴ together are forming a CH₂—CH₂ group.

Another embodiment of the present invention further comprises administration to a subject of the compounds of formula 1, wherein
A is CH₂, O or NMe;
R¹ is selected from
   NHCH₂-R^{1.3};
   R^{1.3} is selected from phenyl, pyrazolyl, isoxazolyl, pyrimidinyl, indolyl or oxadiazolyl, each optionally substituted with one or two residues selected from the group consisting of Me, Et, Pr, cyclopentyl, OMe, OCHF₂;
R² is selected from CH₂-phenyl or CH₂-naphthyl, both optionally substituted with one or two residues selected from the group consisting of CH₃, CF₃, OCF₃, F, Cl, Br, Et; or CH₂-thiophenyl, optionally substituted with one or two residues selected from the group consisting of Cl, Br;
R³ is H;
R⁴ is H;
   or R³ and R⁴ together are forming a CH₂—CH₂ group.

Another embodiment of the present invention further comprises administration to a subject of the compounds of formula 1, wherein
A is CH₂, O or NMe;
R¹ is selected from
   NH-piperidinyl, optionally substituted with pyridinyl;
   NH-cyclohexyl, optionally substituted with one or two residues selected from the group consisting of t-Bu, NHSO₂-phenyl, NHCONH-phenyl, F;
   NH-pyrrolidinyl, optionally substituted with one or two residues selected from the group consisting of SO₂Me, COO-t-Bu;
   piperidinyl, optionally substituted with one or two residues selected from the group consisting of NHSO₂-n-Bu, m-methoxyphenyl;
   dihydro-indolyl, dihydro-isoindolyl, tetrahydro-quinolinyl or tetrahydro-isoquinolinyl, optionally substituted with one or two residues selected from the group consisting of Me, COOMe, CF₃, OMe, NO₂, F, Br;
   a group selected from NHCH(pyridinyl)CH₂COOMe, NHCH(CH₂OMe)-benzoimidazolyl, optionally substituted with Cl;
   or 1-aminocyclopentyl, optionally substituted with Methyl-Oxadiazolyl;
R² is selected from CH₂-phenyl or CH₂-naphthyl, both optionally substituted with one or two residues selected from the group consisting of CH₃, CF₃, OCF₃, F, Cl, Br, Et; or CH₂-thiophenyl, optionally substituted with one or two residues selected from the group consisting of Cl, Br;
R³ is H;
R⁴ is H;
   or R³ and R⁴ together are forming a CH₂—CH₂ group.

Another embodiment of the present invention further comprises administration to a subject of the compounds of formula 1, wherein A is CH₂, O or NMe, R¹ is selected from NHR^{1.2}, NMeR^{1.2}; R² is defined as in Table 1 shown below; R³ is H; R⁴ is and R^{1.2} is selected from
pyridinyl, optionally substituted with one or two residues selected from the group consisting of Me, Et, i-Pr, n-Bu, cyclopropyl, CONR^{1.2.1}R^{1.2.2}, COOMe, COOEt, CONH₂, OMe, Cl, Br CO-pyrrolidinyl, CO-morpholinyl or pyrazolyl, triazolyl, tetrazolyl, isoxazolyl, oxadiazolyl, each optionally substituted Me;
pyrrolyl, optionally substituted with one or two residues selected from the group consisting of Me, Et, COOMe, COOEt;
pyrazolyl, optionally substituted with one or two residues selected from the group consisting of Me, Et, cyclopropyl, COOEt, CO-pyrrolidinyl;
isoxazolyl, optionally substituted with one or two residues selected from the group consisting of t-Bu, COOEt;
thiazolyl, optionally substituted with one or two residues selected from the group consisting of Me, n-Pr, i-Pr, Bu, COOMe, COOEt, CH₂COOEt, CONR^{1.2.1}R^{1.2.2};
thiadiazolyl, optionally substituted with one or two residues selected from the group consisting of COOEt;
benzothiazolyl, indazolyl, dihydro-indolyl, indanyl, tetrahydro-quinolinyl, each optionally substituted with one or two residues selected from the group consisting of NMe₂, CONHMe, =O;
4,5-dihydro-naphtho[2,1-d]thiazole, optionally substituted with NHCOMe,
and
R^{1.2.1} is H or Me;
R^{1.2.2} is H or Me.

Another embodiment of the present invention further comprises administration to a subject of the compounds of formula 1, wherein A is CH₂, O or NMe, R¹ is selected from NHR^{1.2}, NMeR^{1.2}; R² is defined as in Table 1 shown below; R³ is H; R⁴ is and R^{1.2} is selected from
pyridinyl, optionally substituted with one or two residues selected from the group consisting of Me, Et, i-Pr, n-Bu, CONR^{1.2.1}R^{1.2.2}, COOMe, COOEt, CONH₂, OMe, Cl, Br;
pyrrolyl, optionally substituted with one or two residues selected from the group consisting of Me, Et, COOMe, COOEt;
pyrazolyl, optionally substituted with one or two residues selected from the group consisting of Me, Et, cyclopropyl, COOEt, CO-pyrrolidinyl;
isoxazolyl, optionally substituted with one or two residues selected from the group consisting of t-Bu, COOEt;
thiazolyl, optionally substituted with one or two residues selected from the group consisting of Me, n-Pr, i-Pr, Bu, COOMe, COOEt, CONR^{1.2.1}R^{1.2.2};
thiadiazolyl, optionally substituted with one or two residues selected from the group consisting of COOEt;
benzothiazolyl, indazolyl, dihydro-indolyl, indanyl, tetrahydro-quinolinyl, each optionally substituted with one or two residues selected from the group consisting of NMe₂, CONHMe, =O; and

R^{1.2.1} is H or Me;
R^{1.2.2} is H or Me.

Another embodiment of the present invention further comprises administration to a subject of the compounds of formula 1, wherein
A is CH₂, O or NMe, R¹ is selected from NHR^{1.2}, NMeR^{1.2}; R² is defined as in Table 1 shown below; R³ is H; R⁴ is H; R^{1.2} is pyridinyl, optionally substituted with one or two residues selected from the group consisting of Me, Et, i-Pr, n-Bu, CONR^{1.2.1}R^{1.2.2}, COOMe, COOEt, CONH₂, OMe, Cl, Br; R^{1.2.1} is H or Me and R^{1.2.2} is H or Me.
A is CH₂, O or NMe, R¹ is selected from NHR^{1.2}, NMeR^{1.2}; R² is defined as in Table 1 shown below; R³ is H; R⁴ is H; R^{1.2} is pyrrolyl, optionally substituted with one or two residues selected from the group consisting of Me, Et, COOMe, COOEt; R^{1.2.1} is H or Me and R^{1.2.2} is H or Me.
A is CH₂, O or NMe, R¹ is selected from NHR^{1.2}, NMeR^{1.2}; R² is defined as in Table 1 shown below; R³ is H; R⁴ is H; R^{1.2} is pyrazolyl, optionally substituted with one or two residues selected from the group consisting of Me, Et, cyclopropyl, COOEt, CO-pyrrolidinyl_{;} R^{1.2.1} is H or Me and R^{1.2.2} is H or Me.
A is CH₂, O or NMe, R¹ is selected from NHR^{1.2}, NMeR^{1.2}; R² is defined as in Table 1 shown below; R³ is H; R⁴ is H; R^{1.2} is isoxazolyl, optionally substituted with one or two residues selected from the group consisting of t-Bu, COOE; R^{1.2.1} is H or Me and R^{1.2.2} is H or Me.
A is CH₂, O or NMe, R¹ is selected from NHR^{1.2}, NMeR^{1.2}; R² is defined as in Table 1 shown below; R³ is H; R⁴ is H; R^{1.2} is thiazolyl, optionally substituted with one or two residues selected from the group consisting of Me, n-Pr, i-Pr, Bu, COOMe, COOEt, CONR^{1.2.1}R^{1.2.2}; ; R^{1.2.1} is H or Me and R^{1.2.2} is H or Me.
A is CH₂, O or NMe, R¹ is selected from NHR^{1.2}, NMeR^{1.2}; R² is defined as in Table 1 shown below; R³ is H; R⁴ is H; R^{1.2} is thiadiazolyl, optionally substituted with one or two residues selected from the group consisting of COOEt; R^{1.2.1} is H or Me and R^{1.2.2} is H or Me.
A is CH₂, O or NMe, R¹ is selected from NHR^{1.2}, NMeR^{1.2}; R² is defined as in Table 1 shown below; R³ is H; R⁴ is H; R^{1.2} is benzothiazolyl, indazolyl, dihydro-indolyl, indanyl, tetrahydro-quinolinyl, each optionally substituted with one or two residues selected from the group consisting of NMe₂, CONHMe, =O; R^{1.2.1} is H or Me and R^{1.2.2} is H or Me.

Another embodiment of the present invention further comprises administration to a subject of the compounds of formula 1, wherein all groups are defined as above except R^{1.3} is selected from
phenyl, optionally substituted with OCHF₂;
pyrazolyl, optionally substituted with Me or Et;
isoxazolyl, optionally substituted with Pr;
pyrimidinyl, optionally substituted with two OMe;
indolyl;
oxadiazolyl, optionally substituted with cyclopentyl.

Another embodiment of the present invention further comprises administration to a subject of the compounds of formula 1, wherein all groups are defined as above except A is CH₂.

Another embodiment of the present invention further comprises administration to a subject of the compounds of formula 1, wherein all groups are defined as above except A is O.

Another embodiment of the present invention further comprises administration to a subject of the compounds of formula 1, wherein all groups are defined as above except A is NMe.

Another embodiment of the present invention are compounds of formula 1, wherein
A is CH₂, O or NMe;
R¹ is selected from

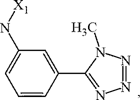

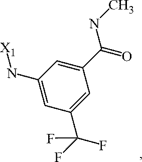

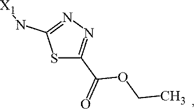

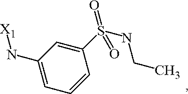

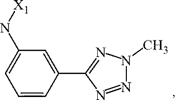

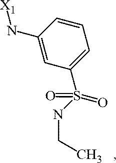

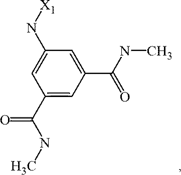

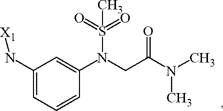

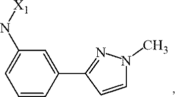

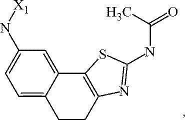

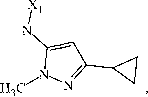

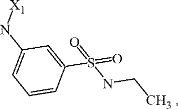

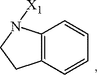

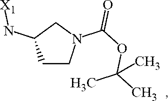

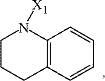

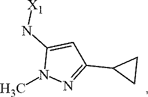

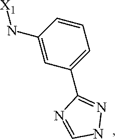

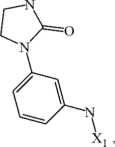

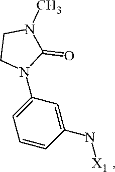

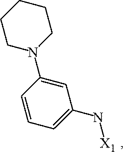

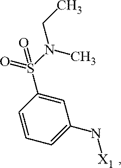

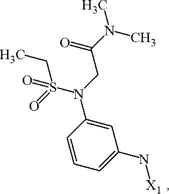

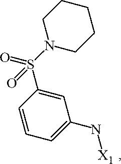

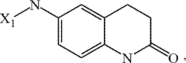

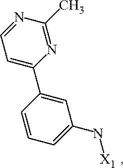

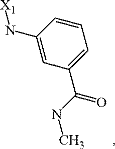

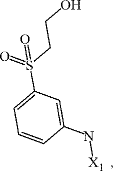

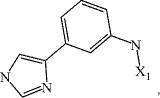

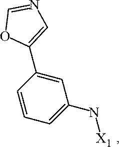

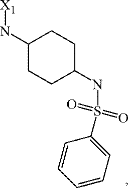

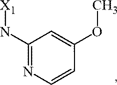

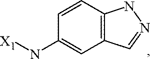

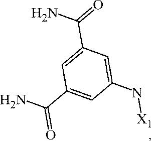

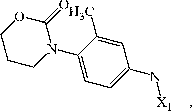

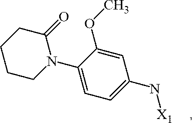

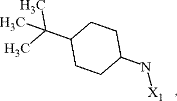

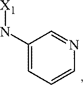

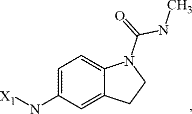

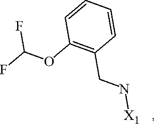

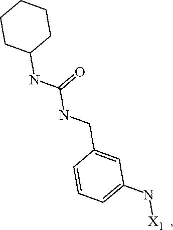

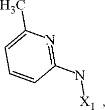

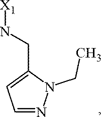

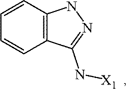

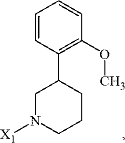

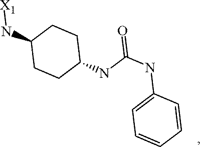

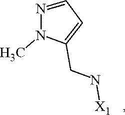

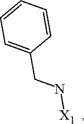

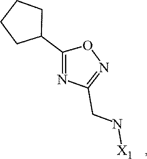

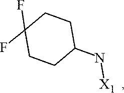

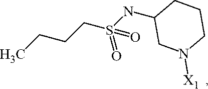

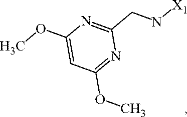

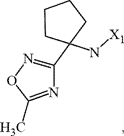

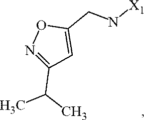

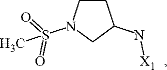

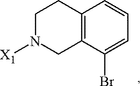

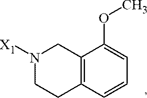

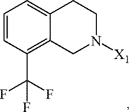

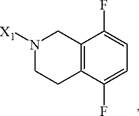

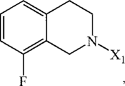

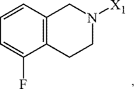

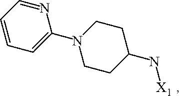

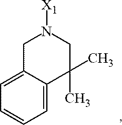

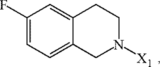

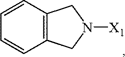

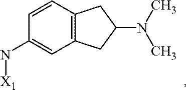

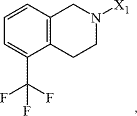

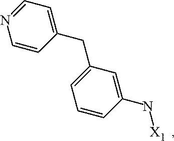

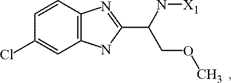

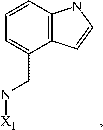

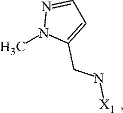

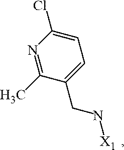

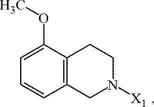

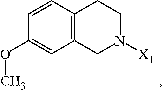

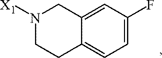

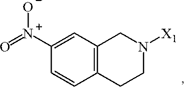

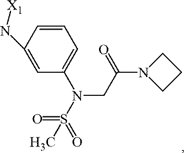

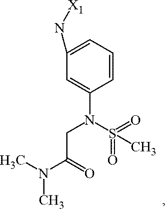

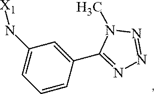

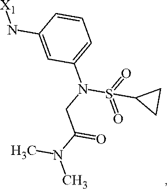

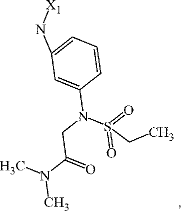

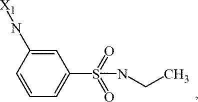

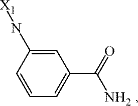

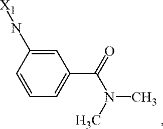

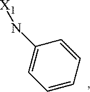

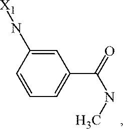

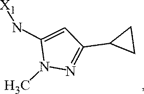

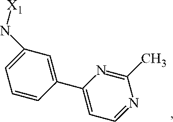

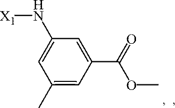

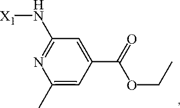

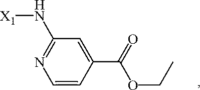

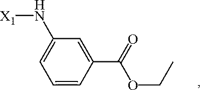

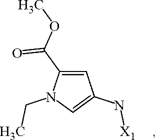

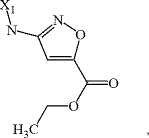

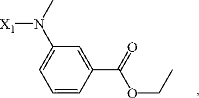

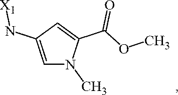

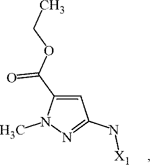

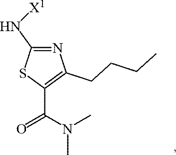

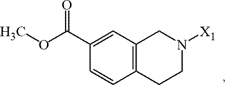

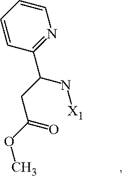

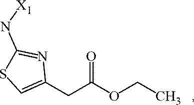

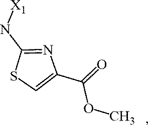

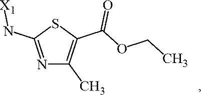

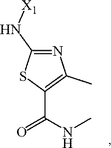

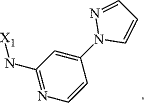

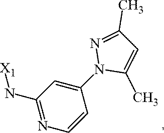

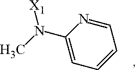

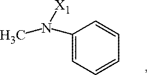

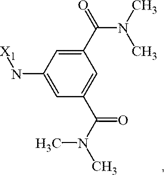

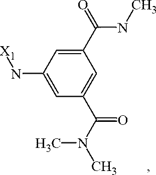

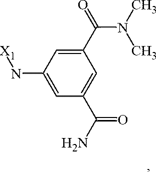

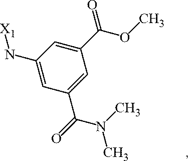

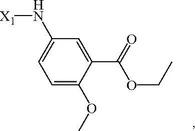

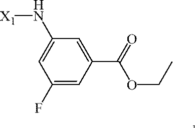

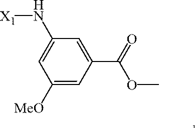

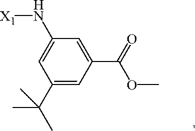

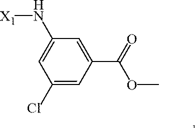

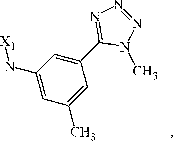

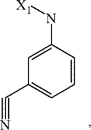

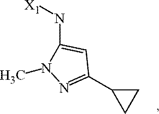

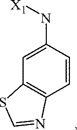

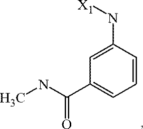

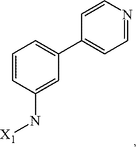

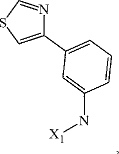

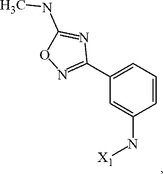

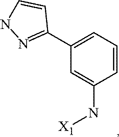

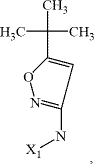

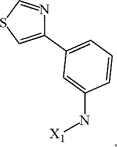

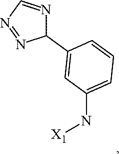

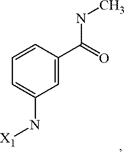

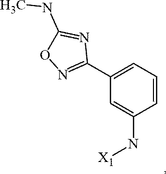

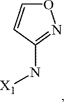

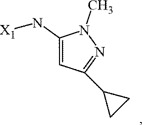

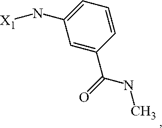

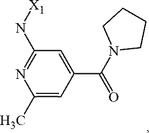

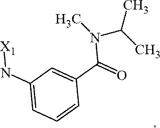

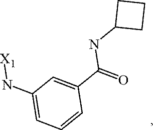

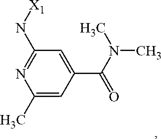

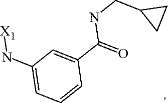

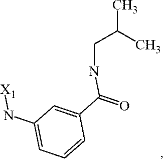

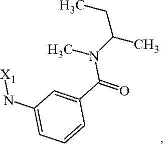

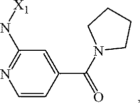

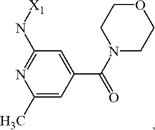

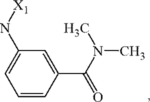

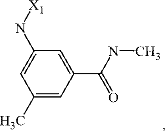

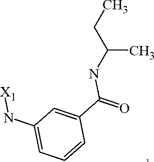

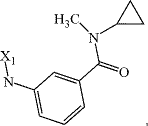

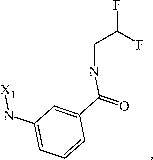

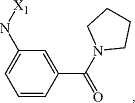

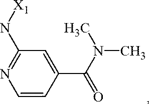

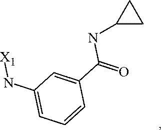

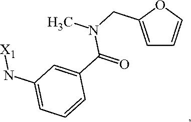

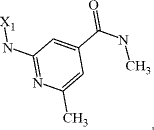

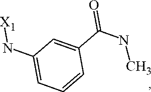

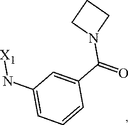

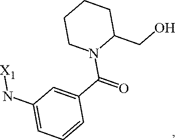

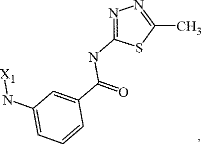

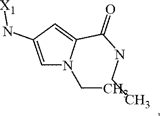

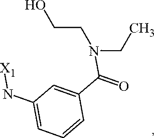

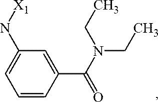

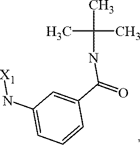

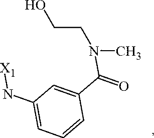

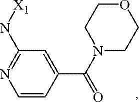

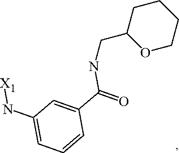

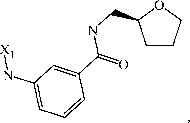

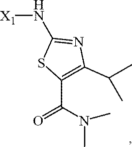

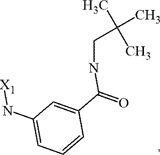

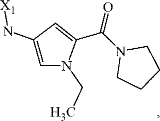

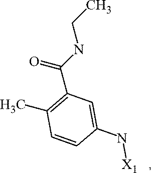

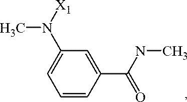

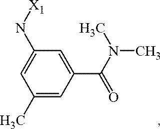

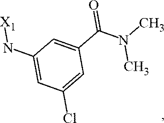

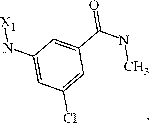

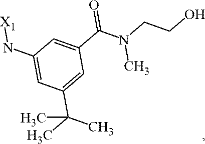

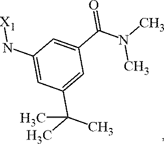

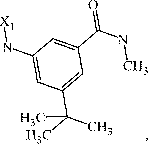

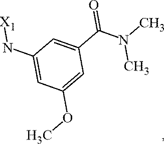

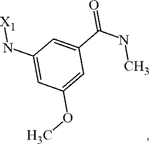

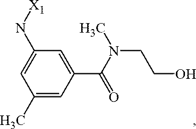

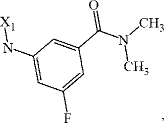

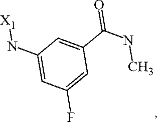

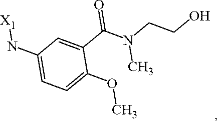

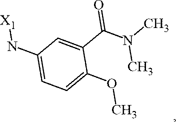

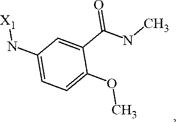

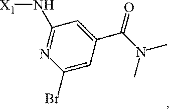

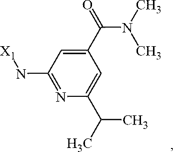

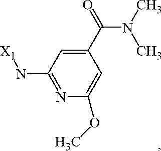

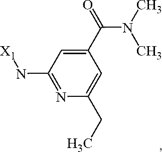

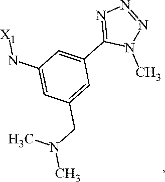

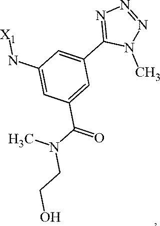

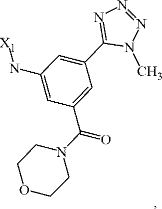

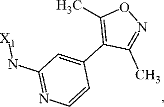

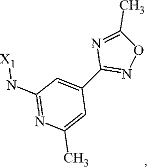

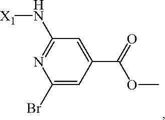

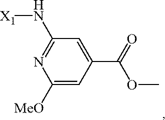

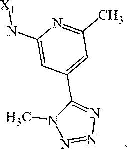

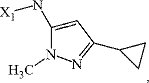

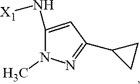

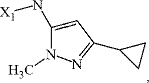

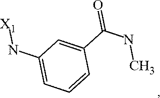

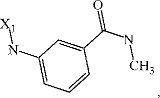

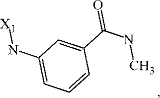

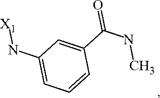

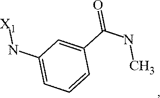

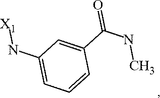

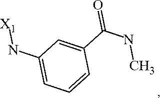

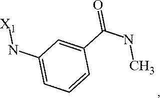

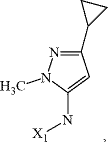

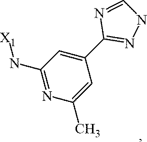

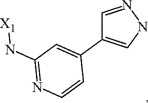

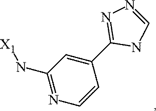

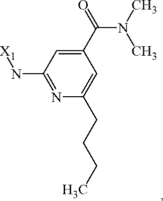

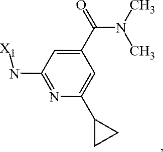

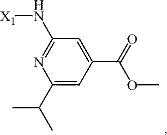

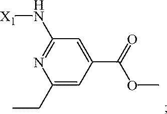

R² is selected from

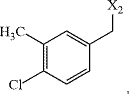

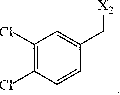

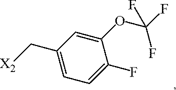

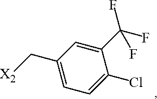

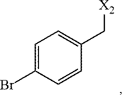

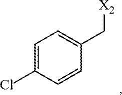

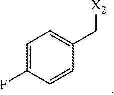

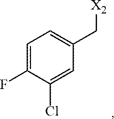

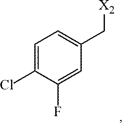

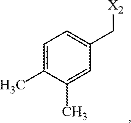

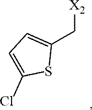

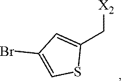

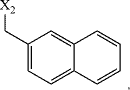

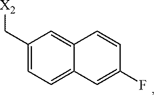

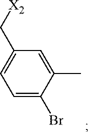

R³ is H;
R⁴ is H;
   or R³ and R⁴ together are forming a CH₂—CH₂ group.

Another embodiment of the present invention are compounds of formula 1, wherein A is defined as above; R³ is H; R⁴ is H; and R² is defined as in Table 1 shown below; and R¹ is selected from

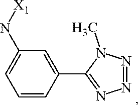

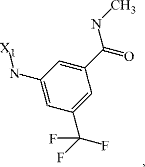

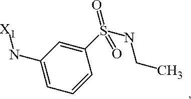

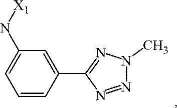

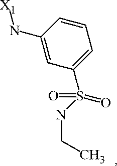

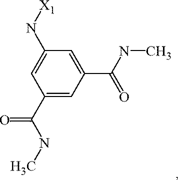

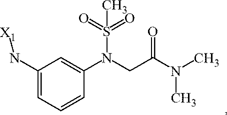

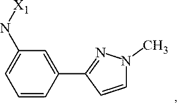

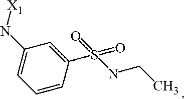

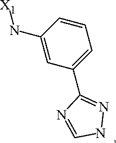

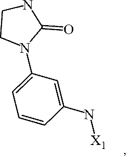

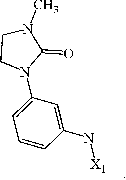

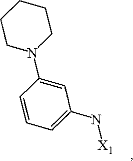

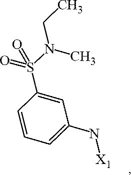

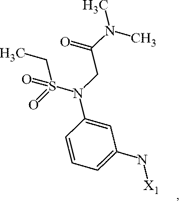

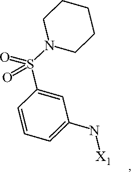

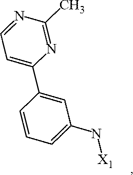

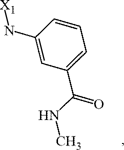

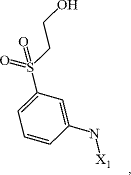

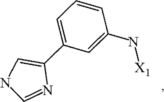

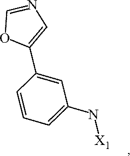

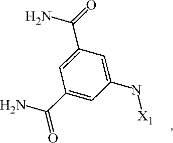

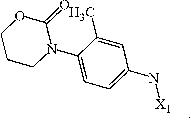

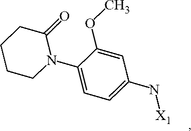

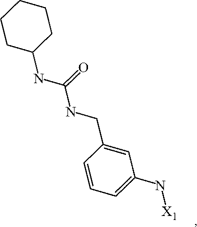

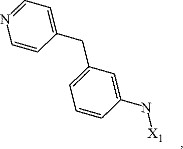

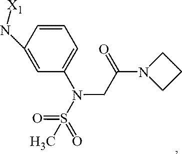

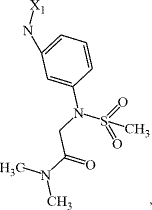

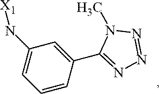

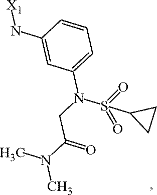

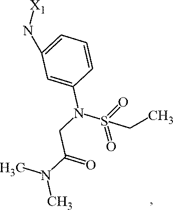

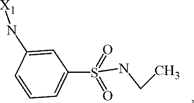

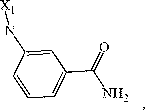

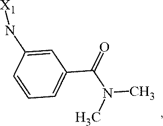

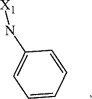

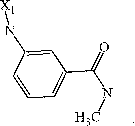

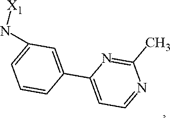

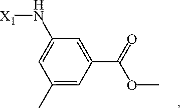

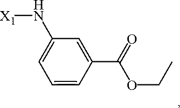

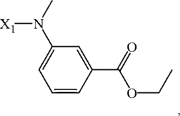

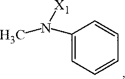

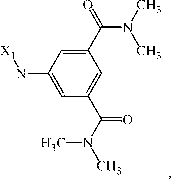

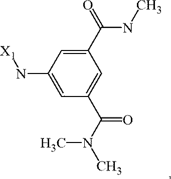

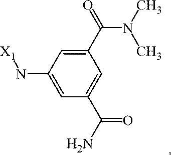

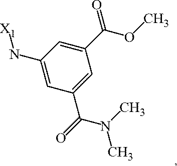

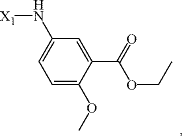

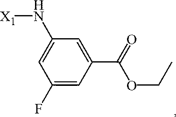

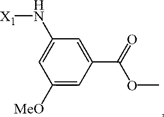

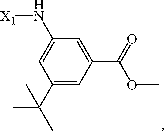

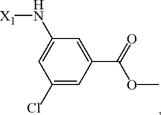

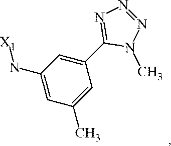

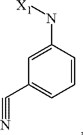

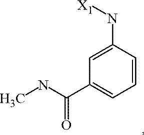

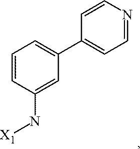

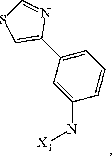

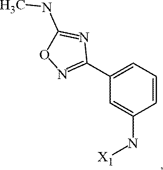

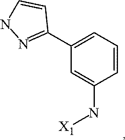

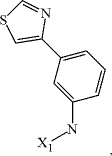

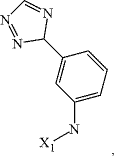

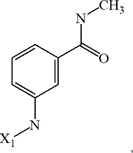

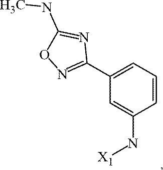

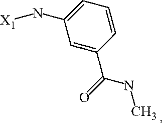

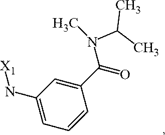

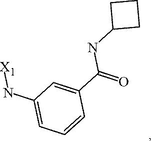

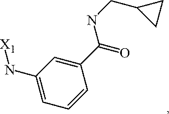

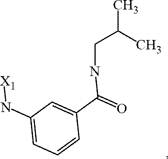

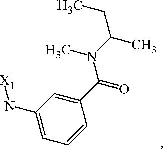

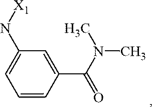

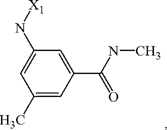

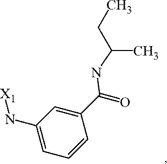

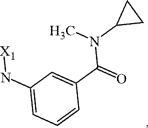

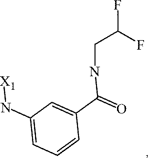

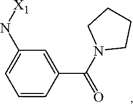

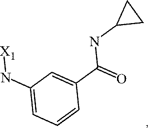

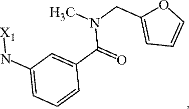

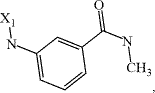

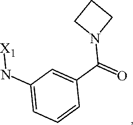

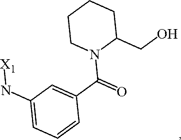

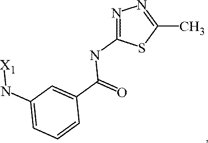

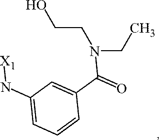

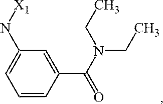

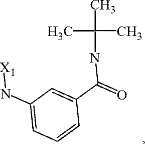

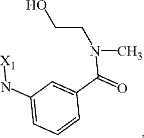

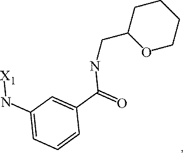

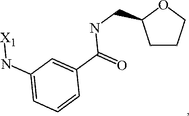

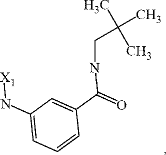

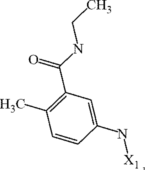

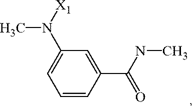

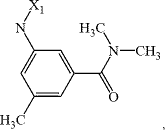

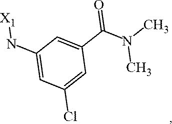

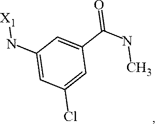

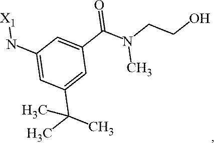

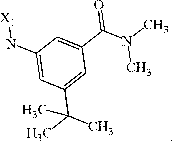

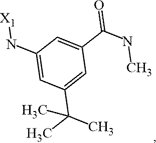

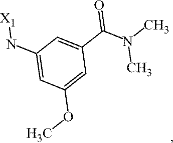

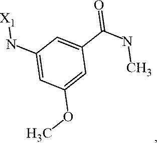

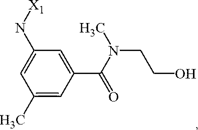

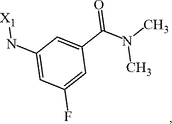

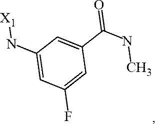

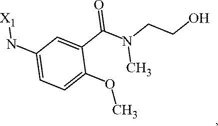

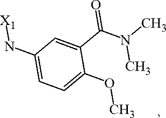

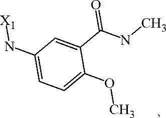

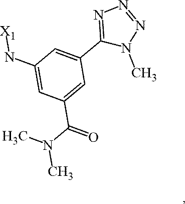

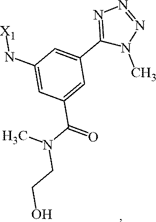

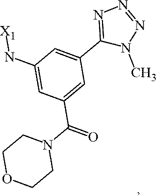

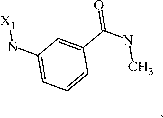

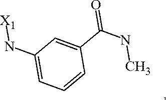

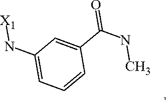

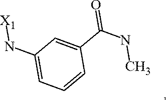

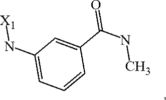

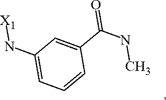

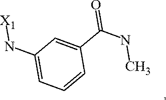

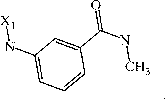

Another embodiment of the present invention further comprises administration to a subject of the compounds of formula 1, wherein A is defined as above; R³ is H; R⁴ is H; and R² is defined as in Table 1 shown below; and R¹ is selected from

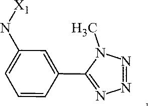

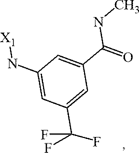

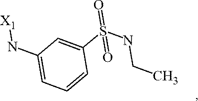

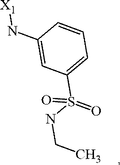

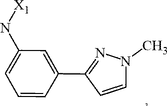

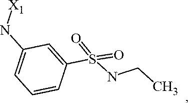

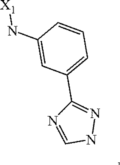

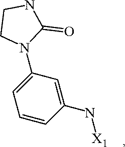

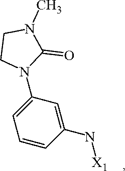

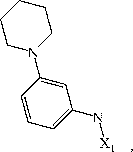

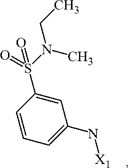

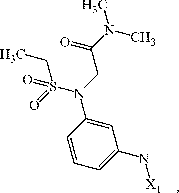

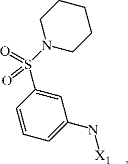

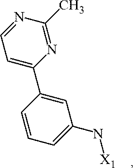

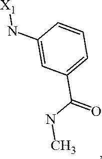

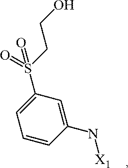

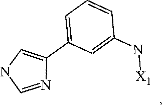

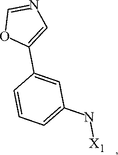

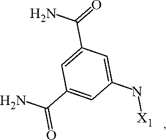

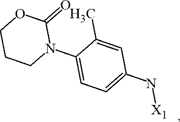

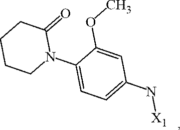

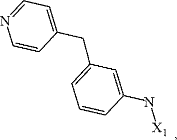

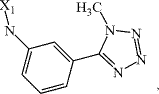

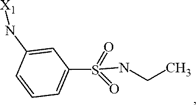

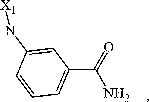

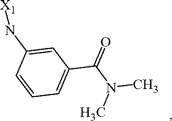

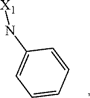

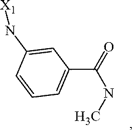

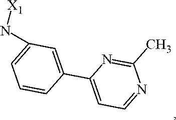

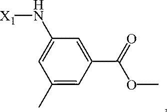

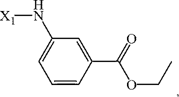

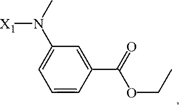

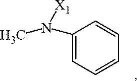

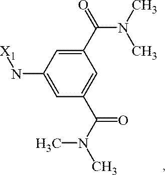

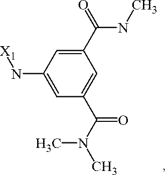

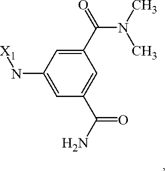

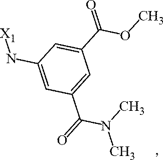

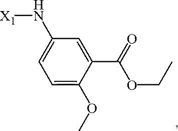

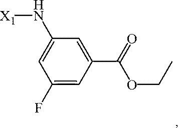

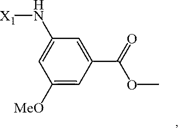

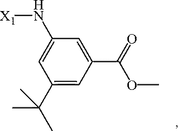

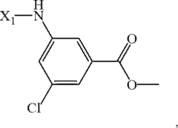

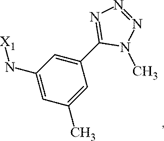

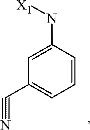

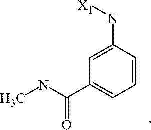

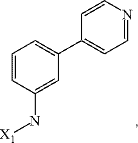

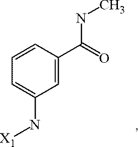

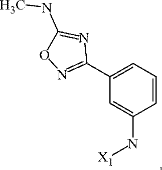

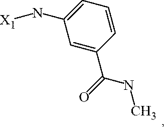

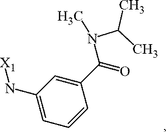

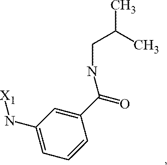

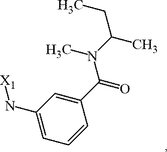

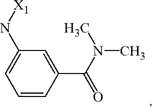

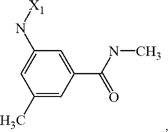

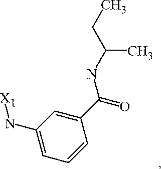

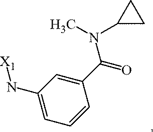

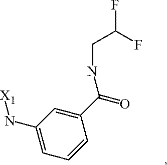

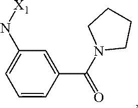

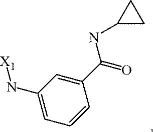

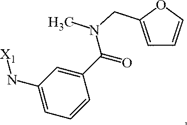

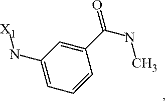

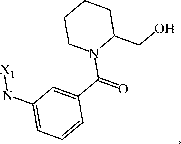

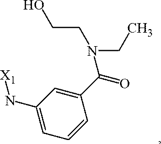

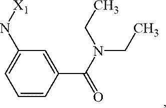

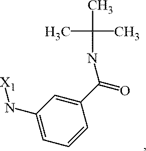

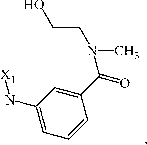

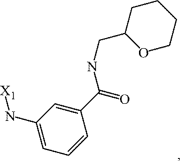

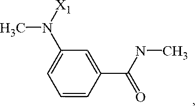

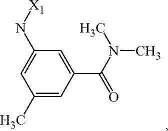

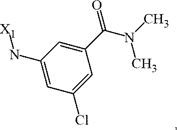

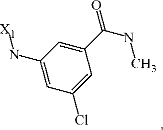

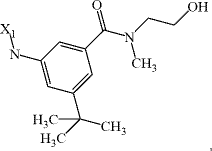

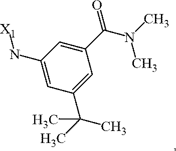

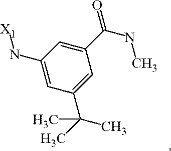

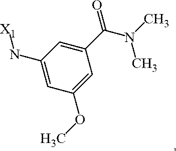

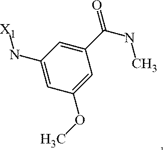

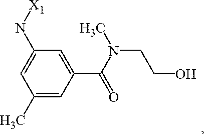

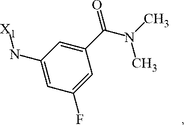

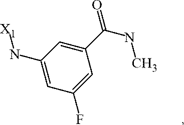

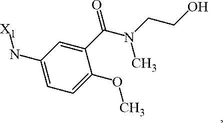

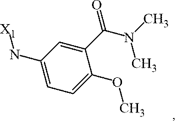

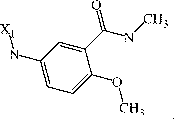

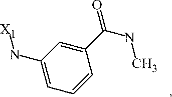

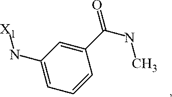

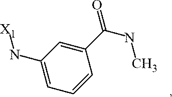

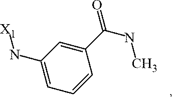

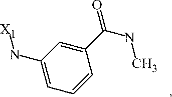

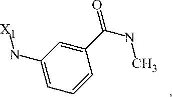

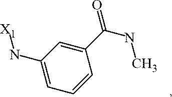

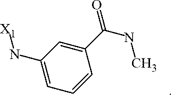

Another embodiment of the present invention further comprises administration to a subject of the compounds of formula 1, wherein A is defined as above; R³ is H; R⁴ is H; and R² is defined as in Table 1 shown below; R¹ is selected from

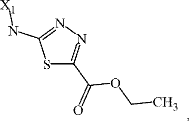

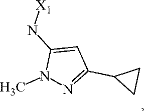

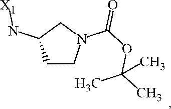

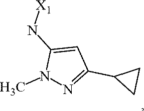

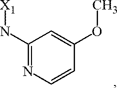

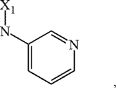

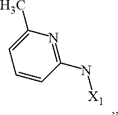

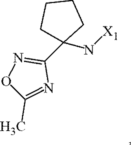

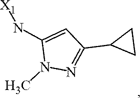

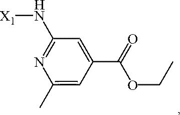

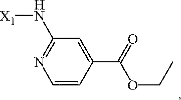

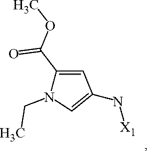

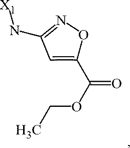

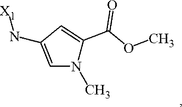

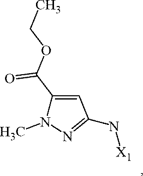

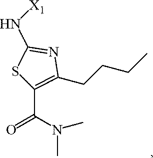

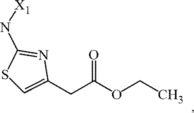

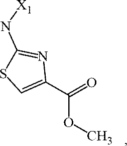

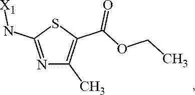

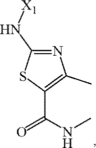

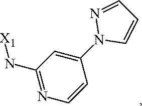

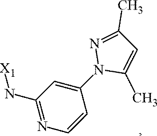

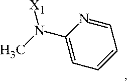

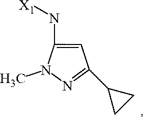

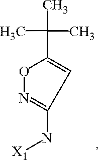

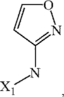

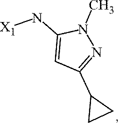

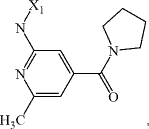

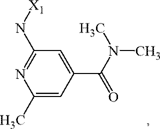

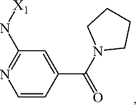

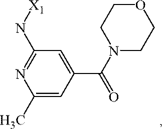

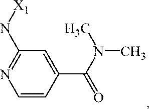

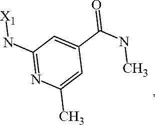

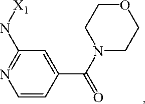

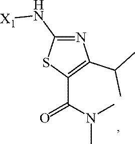

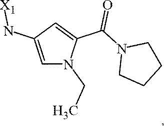

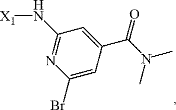

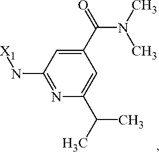

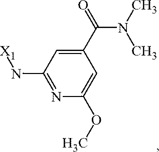

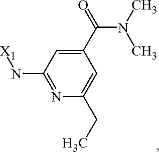

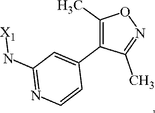

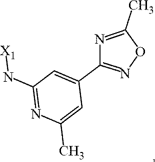

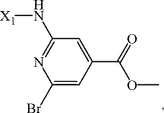

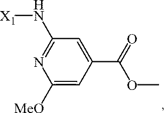

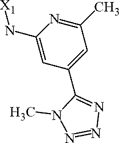

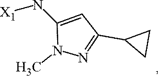

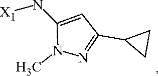

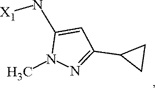

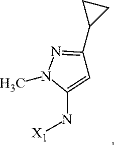

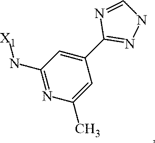

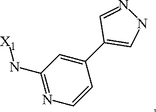

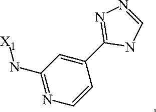

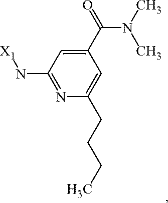

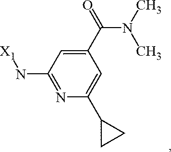

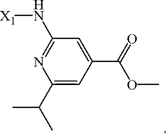

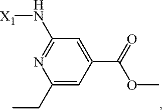

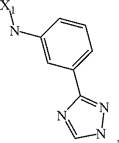

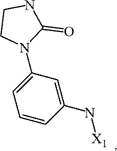

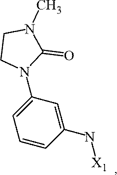

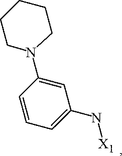

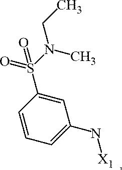

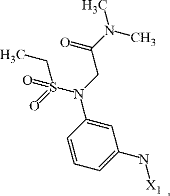

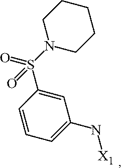

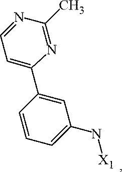

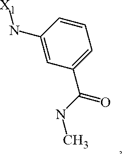

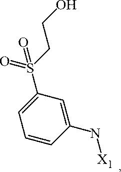

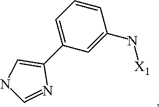

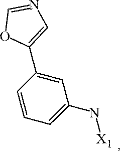

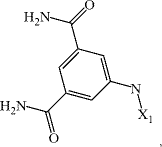

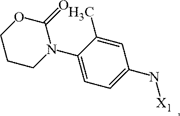

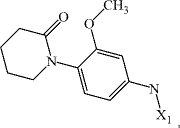

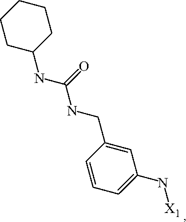

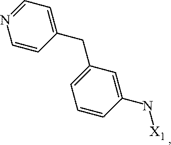

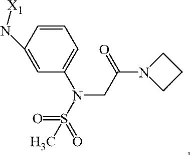

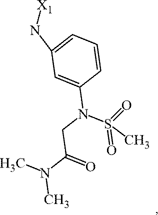

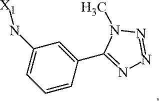

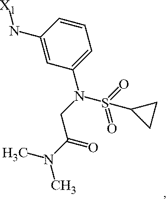

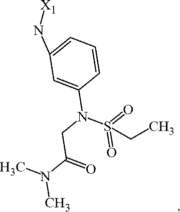

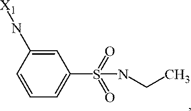

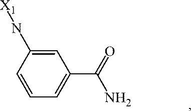

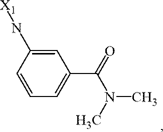

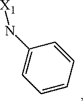

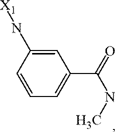

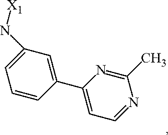

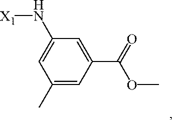

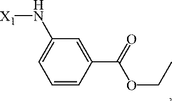

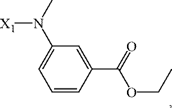

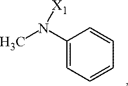

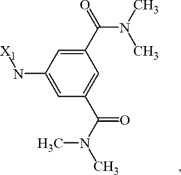

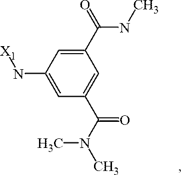

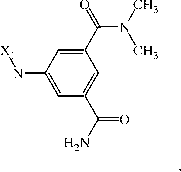

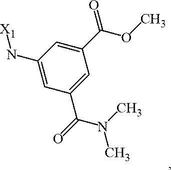

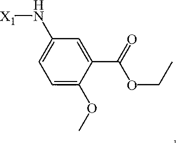

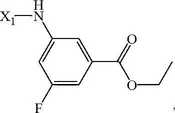

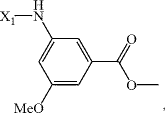

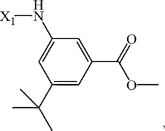

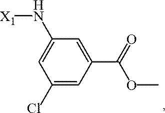

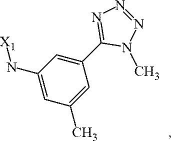

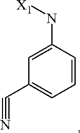

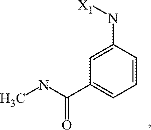

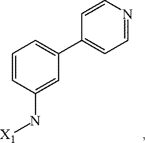

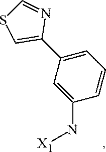

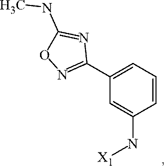

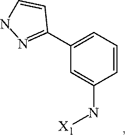

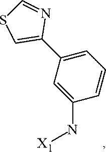

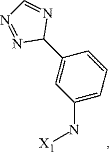

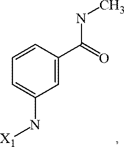

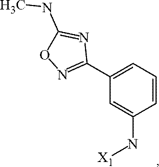

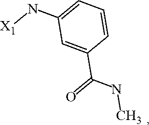

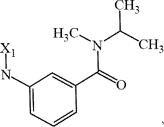

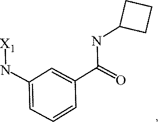

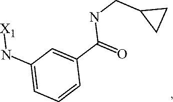

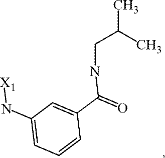

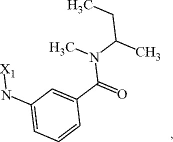

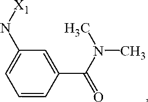

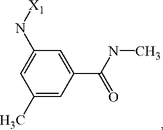

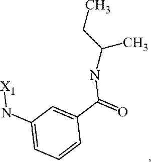

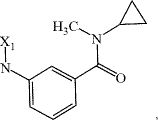

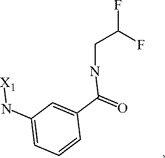

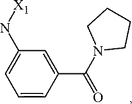

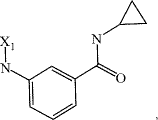

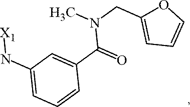

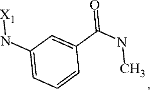

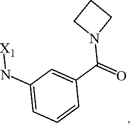

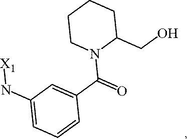

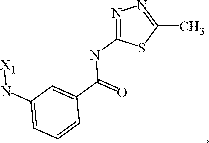

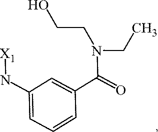

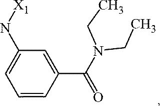

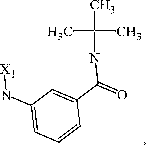

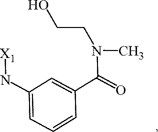

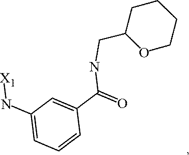

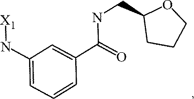

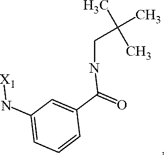

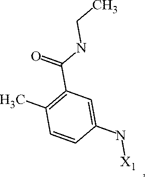

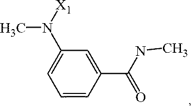

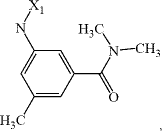

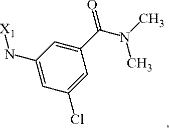

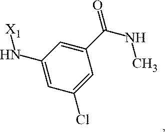

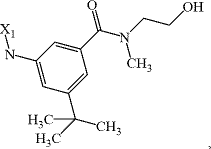

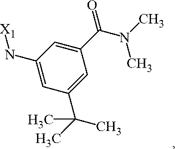

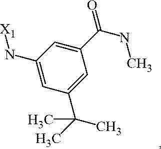

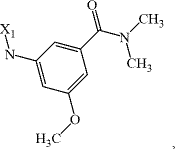

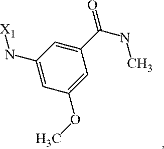

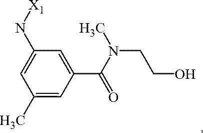

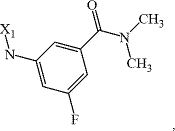

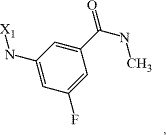

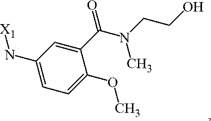

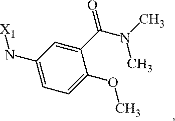

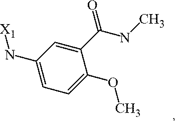

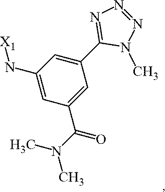

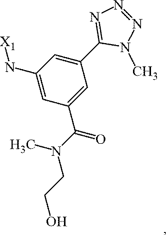

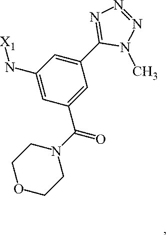

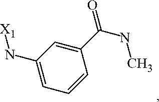

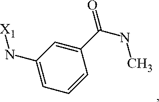

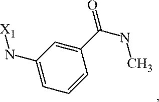

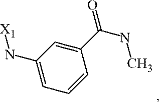

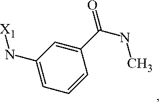

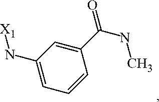

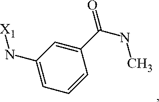

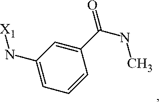

Another embodiment of the present invention further comprises administration to a subject of the compounds of formula 1, wherein A is defined as above; R³ is H; R⁴ is H; and R² is defined as in Table 1 shown below; and R¹ is selected from

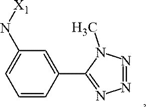

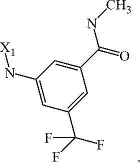

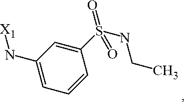

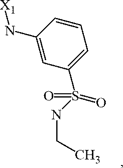

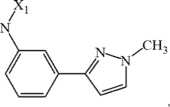

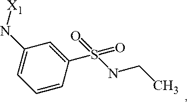

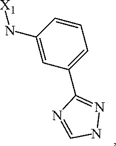

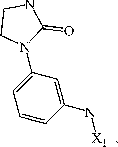

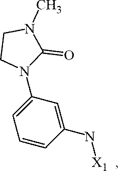

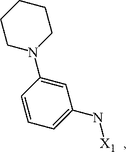

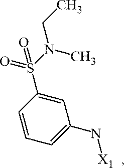

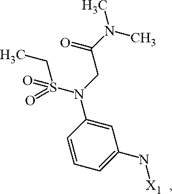

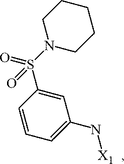

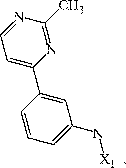

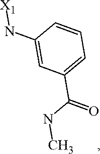

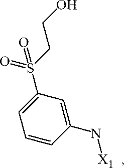

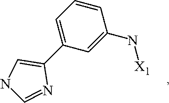

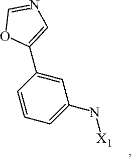

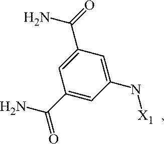

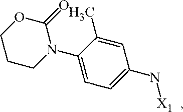

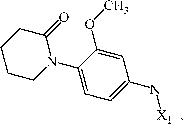

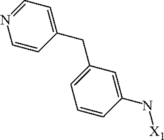

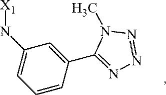

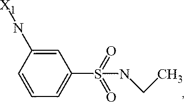

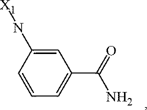

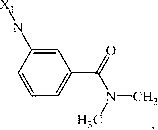

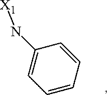

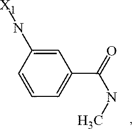

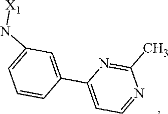

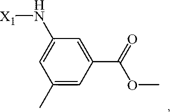

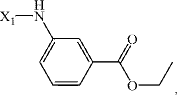

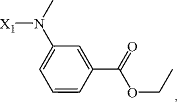

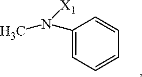

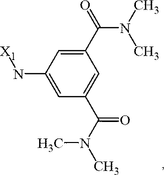

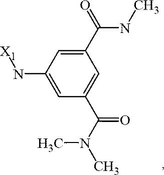

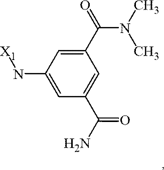

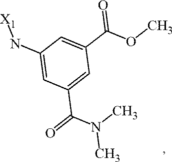

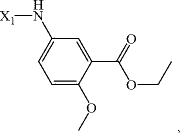

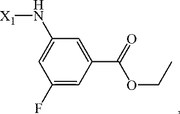

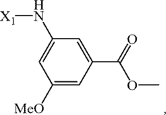

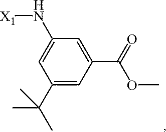

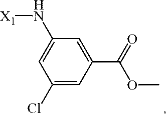

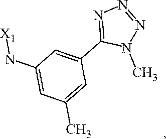

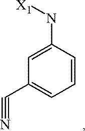

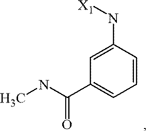

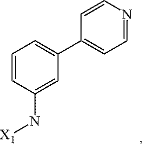

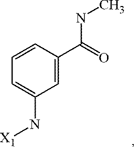

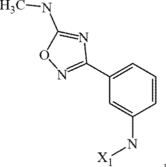

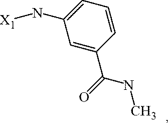

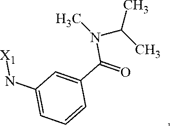

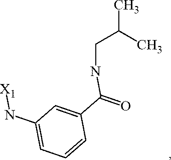

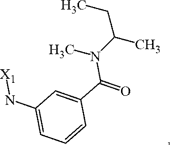

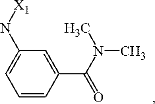

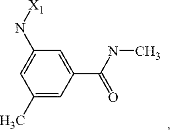

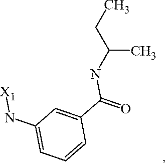

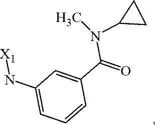

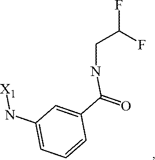

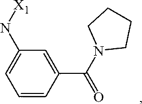

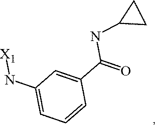

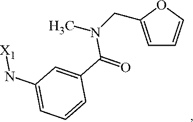

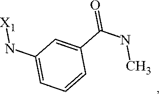

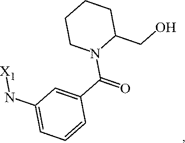

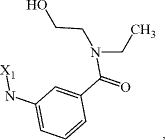

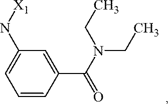

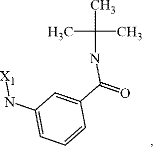

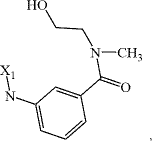

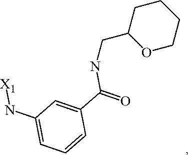

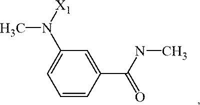

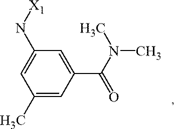

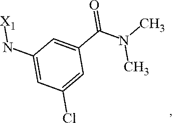

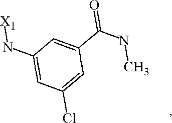

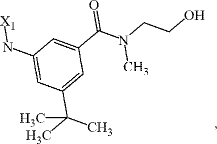

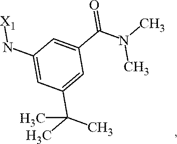

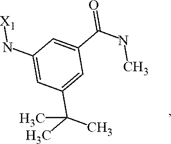

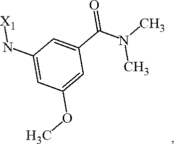

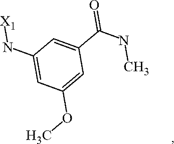

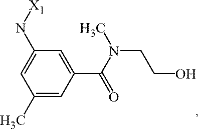

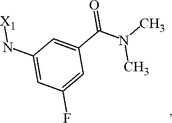

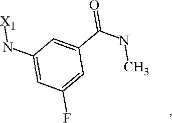

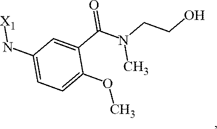

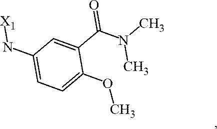

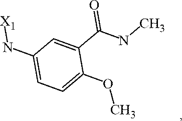

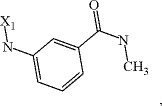

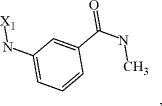

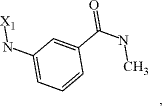

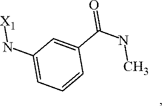

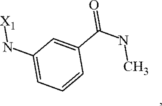

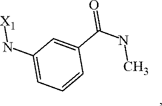

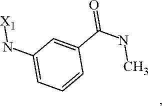

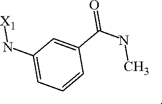

Another embodiment of the present invention further comprises administration to a subject of the compounds of formula 1, wherein A is defined as above; R³ is H; R⁴ is H; and R² is defined as in Table 1 shown below; R¹ is selected from

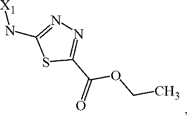

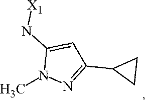

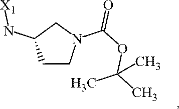

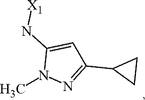

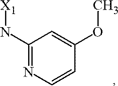

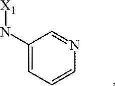

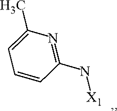

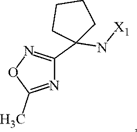

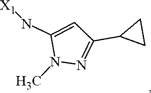

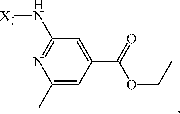

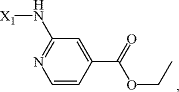

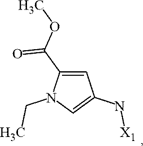

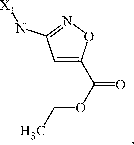

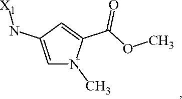

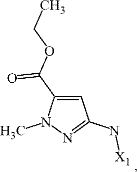

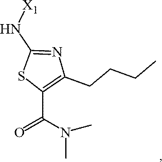

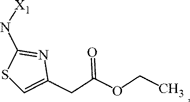

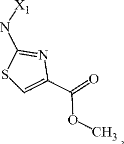

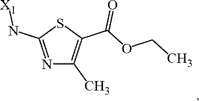

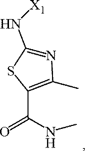

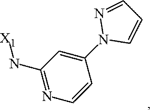

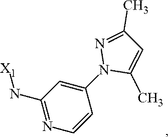

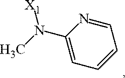

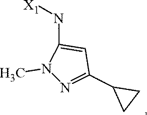

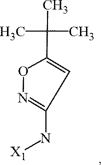

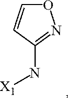

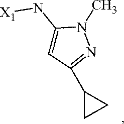

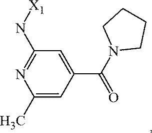

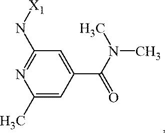

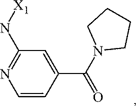

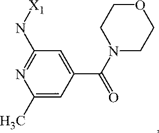

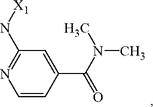

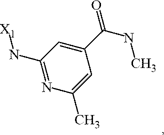

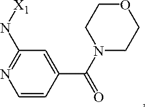

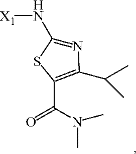

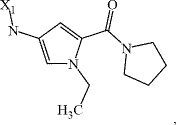

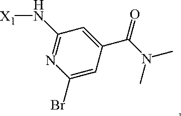

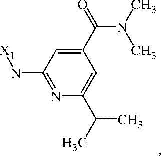

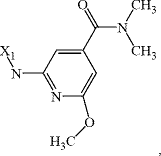

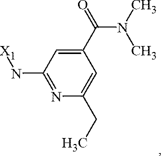

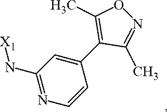

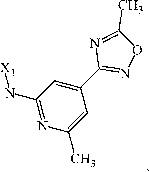

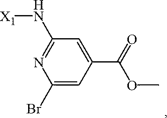

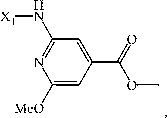

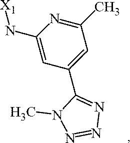

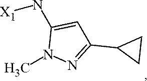

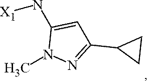

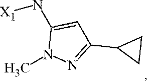

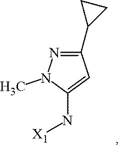

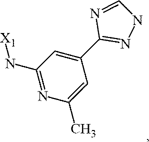

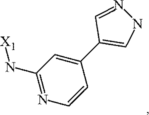

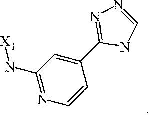

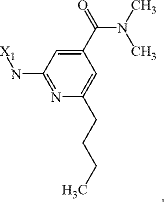

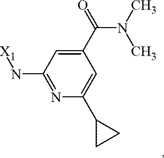

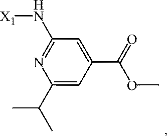

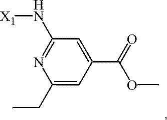

TABLE 1

R² is defined as one of the groups shown below in the definitions 1 to 4

| | | | | | | | |
|---|---|---|---|---|---|---|---|
| Definition 1 | 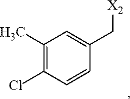 | | 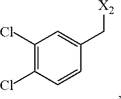 | | | 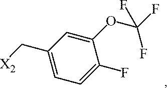 | |
| | 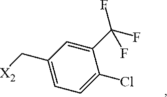 | | 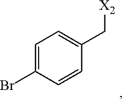 | | 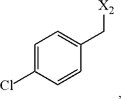 | | 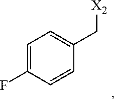 |
| | 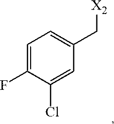 | 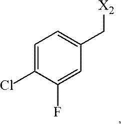 | | 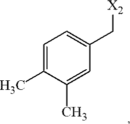 | | 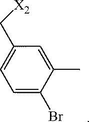 | |
| Definition 2 | | | | 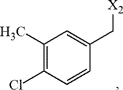 | 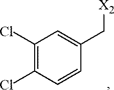 | 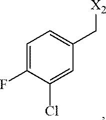 | 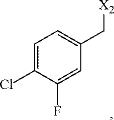 |
| | | | | 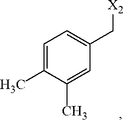 | 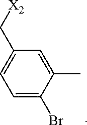 | | |
| Definition 3 | | | | 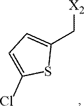 | 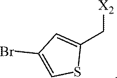 | | |
| Definition 4 | | | | 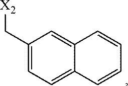 | 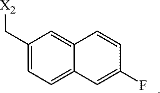 | | |

Another embodiment of the present invention further comprises administration to a subject of the compounds of formula 1, wherein the compounds of formula 1 are present in the form of the individual optical isomers, mixtures of the individual enantiomers or racemates, e.g., in the form of the enantiomerically pure compounds.

Another embodiment of the present invention further comprises administration to a subject of the compounds of formula 1, wherein the compounds of formula 1 are present in the form of the acid addition salts thereof with pharmacologically acceptable acids as well as optionally in the form of the solvates and/or hydrates.

### B. Co-Crystals and Salts

Additional embodiments of the present invention further comprise administration to a subject of the co-crystals of the compounds of formula 2 (below). In general, for groups comprising two or more subgroups in this “Co-Crystals and Salts” section, the first named subgroup is the radical attachment point, for example, the substituent “C₁₋₃-alkyl-aryl” means an aryl group which is bound to a C1-3-alkyl-group, the latter of which is bound to the core or to the group to which the substituent is attached.

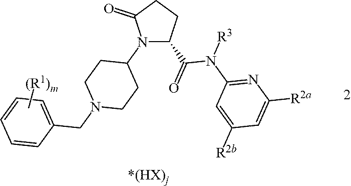

wherein
R¹ is C₁₋₆-alkyl, C₁₋₆-haloalkyl, O-C₁₋₆-haloalkyl, halogen;
m is 1, 2 or 3; and in some instances 1 or 2;
R^{2a} and R^{2b} are each independently selected from H, C₁₋₆-alkyl, C₁₋₆-alkenyl, C₁₋₆-alkynyl, C₃₋₆-cycloalkyl, COO-C₁₋₆-alkyl, O-C₁₋₆-alkyl, CONR^{2b.1}R^{2b.2}, halogen;
   R^{2b.1} is H, C₁₋₆-alkyl, C₀₋₄-alkyl-C₃₋₆-cycloalkyl, C₁₋₆-haloalkyl;
   R^{2b.2} is H, C₁₋₆-alkyl;
   or R^{2b.1} and R^{2b.2} are together a C₃₋₆-alkylene group forming with the nitrogen atom a heterocyclic ring, wherein optionally one carbon atom or the ring is replaced by an oxygen atom
R³ is H, C₁₋₆-alkyl;
X is an anion selected from the group consisting of chloride, bromide, iodide, sulphate, phosphate, methanesulphonate, nitrate, maleate, acetate, benzoate, citrate, salicylate, fumarate, tartrate, dibenzoyltartrate, oxalate, succinate, benzoate and p-toluenesulphonate; and in some instances chloride or dibenzoyltartrate
j is 0, 0.5, 1, 1.5 or 2; and in some instances 1 or 2;
with a co-crystal former selected from the group consisting of orotic acid, hippuric acid, L-pyroglutamic acid, D-pyroglutamic acid, nicotinic acid, L-(+)-ascorbic acid, saccharin, piperazine, 3-hydroxy-2-naphtoic acid, mucic (galactaric) acid, pamoic (embonic) acid, stearic acid, cholic acid, deoxycholic acid, nicotinamide, isonicotinamide, succinamide, uracil, L-lysine, L-proline, D-valine, L-arginine, glycine, in some instances ascorbic acid, mucic acid, pamoic acid, succinamide, nicotinic acid, nicotinamide, isonicotinamide, l-lysine, l-proline.

Another aspect of the present invention further comprises administration to a subject of the co-crystals of the compounds of formula 2, wherein
R^{2a} is H, C₁₋₆-alkyl, C₁₋₆-alkenyl, C₁₋₆-alkynyl, C₃₋₆-cycloalkyl, O-C₁₋₆-alkyl, CONR^{2a.1}R^{2a.2,}
   R^{2a.1} is H, C₁₋₆-alkyl, C₁₋₆-haloalkyl;
   R^{2a.2} is H, C₁₋₆-alkyl;
R^{2b} is H, C₁₋₆-alkyl, C₁₋₆-alkenyl, C₁₋₆-alkynyl, C₃₋₆-cycloalkyl, COO-C₁₋₆-alkyl, O-C₁₋₆-alkyl, CONR^{2b.1}R^{2b.2}, halogen;
   R^{2b.1} is H, C₁₋₆-alkyl, C₀₋₄-alkyl-C₃₋₆-cycloalkyl, C₁₋₆-haloalkyl;
   R^{2b.2} is H, C₁₋₆-alkyl;
   or R^{2b.1} and R^{2b.2} are together a C₃₋₆-alkylene group forming with the nitrogen atom a heterocyclic ring, wherein optionally one carbon atom or the ring is replaced by an oxygen atom and the remaining residues are defined as above.

Another aspect of the present invention further comprises administration to a subject of the co-crystals of the compounds of formula 2, wherein
R^{2a} is H, C₁₋₆-alkyl, C₁₋₆-alkynyl, C₃₋₆-cycloalkyl, O-C₁₋₆-alkyl, CONR^{2a.1}R^{2a.2};
   R^{2a.1} is C₁₋₆-alkyl;
   R^{2a.2} is H;
R^{2b} is H, C₁₋₆-alkyl, O-C₁₋₆-alkyl, CONR^{2b.1}R^{2b.2},
   R^{2b.1} is C₁₋₆-alkyl, C₀₋₄-alkyl-C₃₋₆-cycloalkyl, C₁₋₆-haloalkyl;
   R^{2b.2} is H, C₁₋₆-alkyl;
   or R^{2b.1} and R^{2b.2} are together a C₃₋₆-alkylene group forming with the nitrogen atom a heterocyclic ring, wherein optionally one carbon atom or the ring is replaced by an oxygen atom and the remaining residues are defined as above.

Another aspect of the present invention further comprises administration to a subject of the co-crystals of the compounds of formula 2, wherein
R^{2a} is H, C₁₋₄-alkyl, C₁₋₄-alkynyl, C₃₋₆-cycloalkyl, O-C₁₋₄-alkyl, CONR^{2a.1}R^{2a.2};
   R^{2a.1} is C₁₋₄-alkyl;
   R^{2a.2} is H;
R^{2b} is H, C₁₋₄-alkyl, O-C₁₋₄-alkyl, CONR^{2b.1}R^{2b.2},
   R^{2b.1} is C₁₋₄-alkyl, C₀₋₄-alkyl-C₃₋₆-cycloalkyl, C₁₋₄-haloalkyl;
   R^{2b.2} is H, C₁₋₄-alkyl;
   or R^{2b.1} and R^{2b.2} are together a C₃₋₆-alkylene group forming with the nitrogen atom a heterocyclic ring, wherein optionally one carbon atom or the ring is replaced by an oxygen atom and the remaining residues are defined as above.

Another aspect of the present invention further comprises administration to a subject of the co-crystals of the compounds of formula 2, wherein
R^{2a} is H, C₁₋₄-alkyl,
R^{2b} is H, CONR^{2b.1}R^{2b.2};
   R^{2b.1} is C₁₋₄-alkyl, C₀₋₄-alkyl-C₃₋₆-cycloalkyl, C₁₋₄-haloalkyl;
   R^{2b.2} is H, C₁₋₄-alkyl;
   or R^{2b.1} and R^{2b.2} are together a C₃₋₆-alkylene group forming with the nitrogen atom a heterocyclic ring, wherein optionally one carbon atom or the ring is replaced by an oxygen atom and the remaining residues are defined as above.

Another aspect of the present invention further comprises administration to a subject of the co-crystals of the compounds of formula 2, wherein
R¹ is C₁₋₆-alkyl, C₁₋₆-haloalkyl, O-C₁₋₆-haloalkyl, halogen;
m is 1 or 2;
R^{2a} is H, C₁₋₄-alkyl;
R^{2b} is H, CONR^{2b.1}R^{2b.2};
   R^{2b.1} is C₁₋₄-alkyl, C₀₋₄-alkyl-C₃₋₆-cycloalkyl, C₁₋₄-haloalkyl;
   R^{2b.2} is H, C₁₋₄-alkyl;
   or R^{2b.1} and R^{2b.2} are together a C₃₋₆-alkylene group forming with the nitrogen atom a heterocyclic ring, wherein optionally one carbon atom or the ring is replaced by an oxygen atom
R³ is H, C₁₋₆-alkyl;
X is an anion selected from the group consisting of chloride or dibenzoyltartrate
j is 1 or 2.

Another aspect of the present invention further comprises administration to a subject of the co-crystals of the compounds of formula 2, wherein
R^{2a} is H, C₁₋₄-alkyl; in some instances Methyl, Ethyl, Propyl;
R^{2b} is H, CONR^{2b.1}R^{2b.2};
   R^{2b.1} is C₁₋₄-alkyl; in some instances Methyl, Ethyl, Propyl;
   R^{2b.2} is C₁₋₄-alkyl; in some instances Methyl, Ethyl, Propyl;
   and the remaining residues are defined as above.

Another aspect of the present invention further comprises administration to a subject of the co-crystals of the compounds of formula 2, wherein
R^{2a} is H, C₁₋₄-alkyl; in some instances Methyl, Ethyl, Propyl;
R^{2b} is H, CONR^{2b.1}R^{2b.2};
   R^{2b.1} is C₀₋₄-alkyl-C₃₋₆-cycloalkyl;
   R^{2b.2} is H, C₁₋₄-alkyl; in some instances H, Methyl, Ethyl, Propyl;
   and the remaining residues are defined as above.

Another aspect of the present invention further comprises administration to a subject of the co-crystals of the compounds of formula 2, wherein
R^{2a} is H, C₁₋₄-alkyl; in some instances Methyl, Ethyl, Propyl;
R^{2b} is H, CONR^{2b.1}R^{2b.2};
   R^{2b.1} is C₁₋₄-haloalkyl;
   R^{2b.2} is H, C₁₋₄-alky; in some instances H, Methyl, Ethyl, Propyl;
   and the remaining residues are defined as above.

Another aspect of the present invention further comprises administration to a subject of the co-crystals of the compounds of formula 2, wherein

R^{2b.1} and R^{2b.2} are together a C₃₋₆-alkylene group forming with the nitrogen atom a heterocyclic ring, wherein optionally one carbon atom or the ring is replaced by an oxygen atom and the remaining residues are defined as above.

Another aspect of the present invention further comprises administration to a subject of the co-crystals of the compounds of formula 2, wherein R¹, m, R^{2a}, R^{2b}, R³, X and j are defined as above and the co-crystal former is selected from the group consisting of ascorbic acid, mucic acid, pamoic acid, succinamide, nicotinic acid, nicotinamide, isonicotinamide, l-lysine, l-proline, or hydrates or hydrochlorides of the same.

Another aspect of the present invention further comprises administration to a subject of the co-crystals of the compounds of formula 2a, wherein R^{2a}, R^{2b}, R³, X and j are defined as above

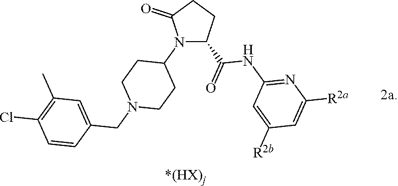

Another aspect of the present invention further comprises administration to a subject of the co-crystals of the compounds of formula 2a, wherein
R^{2a} is H, C₁₋₄-alkyl; in some instances Methyl, Ethyl, Propyl;
R^{2b} is H, CONR^{2b.1}R^{2b.2};
   R^{2b.1} is C₁₋₄-alkyl; in some instances Methyl, Ethyl, Propyl;
   R^{2b.2} is C₁₋₄-alkyl; in some instances Methyl, Ethyl, Propyl; and the remaining residues are defined as above.

Another aspect of the present invention further comprises administration to a subject of the co-crystals of the compounds of formula 2a, wherein
R^{2a} is H, C₁₋₄-alkyl; in some instances Methyl, Ethyl, Propyl;
R^{2b} is H, CONR^{2b.1}R^{2b.2};
   R^{2b.1} is C₀₋₄-alkyl-C₃₋₆-cycloalkyl;
   R^{2b.2} is H, C₁₋₄-alkyl; in some instances H, Methyl, Ethyl, Propyl; and the remaining residues are defined as above.

Another aspect of the present invention further comprises administration to a subject of the co-crystals of the compounds of formula 2a, wherein
R^{2a} is H, C₁₋₄-alkyl; in some instances Methyl, Ethyl, Propyl;
R^{2b} is H, CONR^{2b.1}R^{2b.2};
   R^{2b.1} is C₁₋₄-haloalkyl;
   R^{2b.2} is H, C₁₋₄-alky; in some instances H, Methyl, Ethyl, Propyl; and the remaining residues are defined as above.

Another aspect of the present invention further comprises administration to a subject of the co-crystals of the compounds of formula 2a, wherein
R^{2b.1} and R^{2b.2} are together a C₃₋₆-alkylene group forming with the nitrogen atom a heterocyclic ring, wherein optionally one carbon atom or the ring is replaced by an oxygen atom
and the remaining residues are defined as above.

The free bases of compounds of formula 2 (j = 0) are often amorphous and are used for a process of manufacturing co-crystal, nevertheless salts of compounds of formula 2 are employed in some instances for a process of manufacturing co-crystal. Thus, another aspect of the invention are salts of compounds of formula 2 wherein R¹, m, R^{2a}, R^{2b}, R³ are defined as for the co-crystals above and
X is an anion selected from the group consisting of chloride, bromide, iodide, sulphate, phosphate, methanesulphonate, nitrate, maleate, acetate, benzoate, citrate, salicylate, fumarate, tartrate, dibenzoyltartrate, oxalate, succinate, benzoate and p-toluenesulphonate; in some instances chloride, or dibenzoyltartrate
j is 0, 0.5, 1, 1.5 or 2; in some instances 1 or 2.

Another aspect of the present invention further comprises administration to a subject of the co-crystals of the compounds of formula 2, wherein R¹, m, R^{2a}, R^{2b}, R³ are defined as for the co-crystals above and
X is an anion selected from the group consisting of chloride or dibenzoyltartrate
j is 1 or 2.

Another aspect of the present invention further comprises administration to a subject of the salts of the compounds of formula 2, wherein R¹, m, R^{2a}, R^{2b}, R³ are defined as for the salts above and X is chloride and j is 2.

Another aspect of the present invention further comprises administration to a subject of the salts of the compounds of formula 2, wherein R¹, m, R^{2a}, R^{2b}, R³ are defined as for the salts above and X is dibenzoyltartrate and j is 1.

Another aspect of the present invention further comprises administration to a subject of the salts of the compounds of formula 2a, wherein R^{2a}, R^{2b}, R³, X and j are defined as above

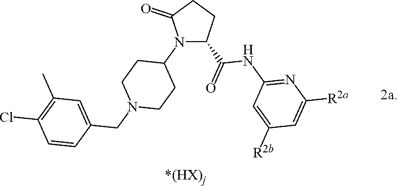

Another aspect of the present invention further comprises administration to a subject of the salts of the compounds of formula 2a, wherein
R^{2a} is H, C₁₋₄-alkyl; in some instances Methyl, Ethyl, Propyl;
R^{2b} is H, CONR^{2b.1}R^{2b.2};
   R^{2b.1} is C₁₋₄-alkyl; in some instances Methyl, Ethyl, Propyl;
   R^{2b.2} is C₁₋₄-alkyl; in some instances Methyl, Ethyl, Propyl;
and the remaining residues are defined as above.

Another aspect of the present invention further comprises administration to a subject of the salts of the compounds of formula 2a, wherein
R^{2a} is H, C₁₋₄-alkyl; in some instances Methyl, Ethyl, Propyl;
R^{2b} is H, CONR^{2b.1}R^{2b.2};
   R^{2b.1} is C₀₋₄-alkyl-C₃₋₆-cycloalkyl;
   R^{2b.2} is H, C₁₋₄-alkyl; in some instances H, Methyl, Ethyl, Propyl;
and the remaining residues are defined as above.

Another aspect of the present invention further comprises administration to a subject of the salts of the compounds of formula 2a, wherein
R^{2a} is H, C₁₋₄-alkyl; in some instances Methyl, Ethyl, Propyl;
R^{2b} is H, CONR^{2b.1}R^{2b.2};
   R^{2b.1} is C₁₋₄-haloalkyl;
   R^{2b.2} is H, C₁₋₄-alky; in some instances H, Methyl, Ethyl, Propyl;
and the remaining residues are defined as above.

Another aspect of the present invention further comprises administration to a subject of the salts of the compounds of formula 2a, wherein

R^{2b.1} and R^{2b.2} are together a C₃₋₆-alkylene group forming with the nitrogen atom a heterocyclic ring, wherein optionally one carbon atom or the ring is replaced by an oxygen atom and the remaining residues are defined as above.

Another aspect of the present invention further comprises administration to a subject of the salts of the compounds of formula 2a, wherein R¹, m, R^{2a}, R^{2b}, R³ are defined as for the salts above and X is chloride and j is 2.

Another aspect of the present invention further comprises administration to a subject of the salts of the compounds of formula 2a, wherein R¹, m, R^{2a}, R^{2b}, R³ are defined as for the salts above and X is dibenzoyltartrate and j is 1. Another aspect of the invention are salts of compounds of formula 2a, wherein R¹, m, R^{2a}, R^{2b}, R³ are defined as for the salts above and X is (S)-(S)-(+)-2,3-dibenzoyl-tartrate and j is 1.

### C. Formulations

Additional embodiments of the present invention further comprise administration to a subject of a pharmaceutical composition containing compounds of formula 3

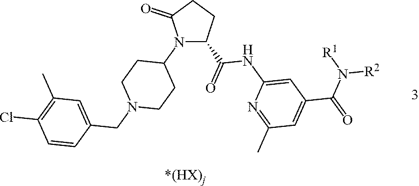

wherein
R¹ is H, C₁₋₆-alkyl, C₀₋₄-alkyl-C₃₋₆-cycloalkyl, C₁₋₆-haloalkyl;
R² is H, C₁₋₆-alkyl;
X is an anion selected from the group consisting of chloride or ½ dibenzoyltartrate
j is 1 or 2.

An embodiment of the present invention further comprises administration to a subject of a pharmaceutical composition containing compounds of formula 3 wherein
R¹ is H, C₁₋₆-alkyl;
R² is H, C₁₋₆-alkyl;
X is an anion selected from the group consisting of chloride or ½ dibenzoyltartrate
j is 1 or 2.

An embodiment of the present invention further comprises administration to a subject of a pharmaceutical composition containing compounds of formula 3 wherein
R¹ is H, Methyl, Ethyl, Propyl, Butyl;
R² is H, Methyl, Ethyl, Propyl, Butyl;
X is an anion selected from the group consisting of chloride or ½ dibenzoyltartrate, such as chloride;
j is 1 or 2, in some instances 2.

An embodiment of the present invention further comprises administration to a subject of a pharmaceutical composition containing compounds of formula 3 wherein
R¹ is H, Methyl, Ethyl, Propyl, Butyl;
R² is H, Methyl;
X is an anion selected from the group consisting of chloride or ½ dibenzoyltartrate, such as chloride;
j is 1 or 2, in some instances 2.

An embodiment of the present invention further comprises administration to a subject of a pharmaceutical composition containing compounds of formula 3 wherein
R¹ is H, Methyl;
R² is H, Methyl;
X is an anion selected from the group consisting of chloride or ½ dibenzoyltartrate, such as chloride;
j is 1 or 2, in some instances 2.

An embodiment of the present invention further comprises administration to a subject of a pharmaceutical composition containing compounds described in Table 2 as a hydrochloride. An additional embodiment of the present invention further comprises administration to a subject of a pharmaceutical composition containing compounds describe in Table 2 as a di-hydrochloride.

TABLE 2

| # | Structure |
|---|---|
| 1 | 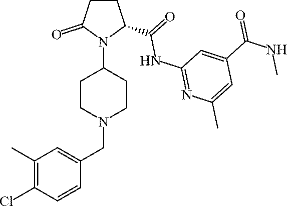 |
| 2 | 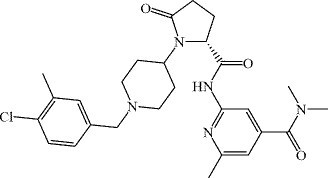 [SMILES: Cc1cc(C(=O)N(C)C)cc(NC(=O)[C@H]2CCC(=O)N2C2CCN(Cc3ccc(Cl)c(C)c3)CC2)n1] |
| 3 | 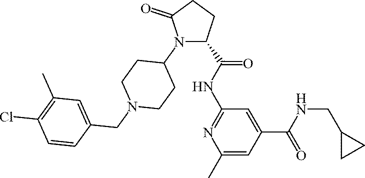 [SMILES: Cc1cc(C(=O)NCC2CC2)cc(NC(=O)[C@H]2CCC(=O)N2C2CCN(Cc3ccc(Cl)c(C)c3)CC2)n1] |
| 4 | 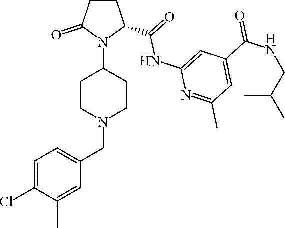 [SMILES: Cc1cc(C(=O)NCC(C)C)cc(NC(=O)[C@H]2CCC(=O)N2C2CCN(Cc3ccc(Cl)c(C)c3)CC2)n1] |
| 5 | 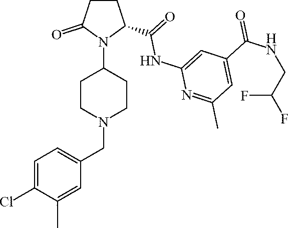 [SMILES: Cc1cc(C(=O)NCC(F)F)cc(NC(=O)[C@H]2CCC(=O)N2C2CCN(Cc3ccc(Cl)c(C)c3)CC2)n1] |
| 6 | 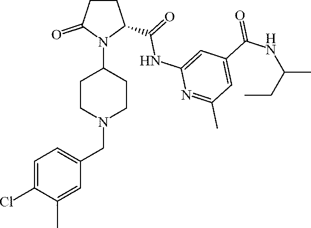 [SMILES: CCC(C)NC(=O)c1cc(C)nc(NC(=O)[C@H]2CCC(=O)N2C2CCN(Cc3ccc(Cl)c(C)c3)CC2)c1] |
| 7 | 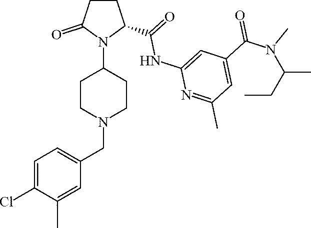 [SMILES: CCC(C)N(C)C(=O)c1cc(C)nc(NC(=O)[C@H]2CCC(=O)N2C2CCN(Cc3ccc(Cl)c(C)c3)CC2)c1] |
| 8 | 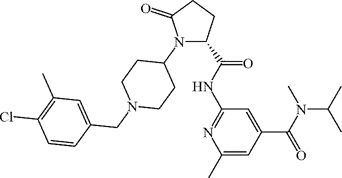 [SMILES: Cc1cc(C(=O)N(C)C(C)C)cc(NC(=O)[C@H]2CCC(=O)N2C2CCN(Cc3ccc(Cl)c(C)c3)CC2)n1] |
| 9 | 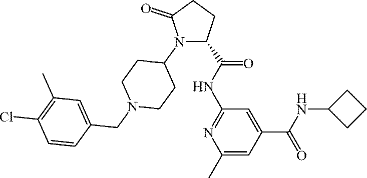 [SMILES: Cc1cc(C(=O)NC2CCC2)cc(NC(=O)[C@H]2CCC(=O)N2C2CCN(Cc3ccc(Cl)c(C)c3)CC2)n1] |
| 10 | 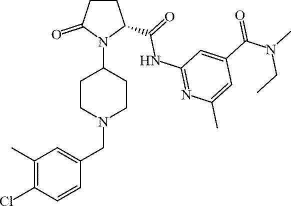 |

Another object of the present invention is administration to a subject of a pharmaceutical dosage form of the compounds described above, wherein the dosage is an orally deliverable dosage form. Yet another object of the present invention is administration to a subject of a pharmaceutical dosage form of the compounds described above, which is in the form of a tablet, capsule, pellets, powder or granules. Another object of the present invention is administration to a subject of the pharmaceutical dosage forms described above for use as medicament.

Yet another object of the present invention is the use of the above pharmaceutical dosage forms for the preparation of a medicament for the treatment of a skin disorder dition selected from xerosis, dermatitis, dyshydrotic dermatitis, drug reactions, urticaria, atopic dermatitis/neurodermatitis, seborrheic dermatitis, psoriasis, palmoplantar pustulosis, lichen planus, pityriasis rubra pilaris, darier disease, Hailey-Hailey disease, Grover’s disease, polymorphic light eruptions, bullous pemphigoid, acquired epidermolysis bullosa, dermatitis herpetiformis, pemphigus vulgaris, dermatomyositis, systemic sclerosis, Sjögren syndrome, Herpes simplex, Herpes zoster, tineas, candidal intertrigo; malassezia folliculitis, Ofuji’s disease, scabies, lice, cutaneous larva migrans, insect bites/arthropod reactions, rosacea, mastocytosis, cutaneous lymphomas, mycosis fungoides, and Sezary syndrome.

Another object of the present invention is a process for the treatment and/or prevention of a disease or symptoms selected from xerosis, dermatitis, dyshydrotic dermatitis, drug reactions, urticaria, atopic dermatitis/neurodermatitis, seborrheic dermatitis, psoriasis, palmoplantar pustulosis, lichen planus, pityriasis rubra pilaris, darier disease, Hailey-Hailey disease, Grover’s disease, polymorphic light eruptions, bullous pemphigoid, acquired epidermolysis bullosa, dermatitis herpetiformis, pemphigus vulgaris, dermatomyositis, systemic sclerosis, Sjögren syndrome, Herpes simplex, Herpes zoster, tineas, candidal intertrigo; malassezia folliculitis, Ofuji’s disease, scabies, lice, cutaneous larva migrans, insect bites/arthropod reactions, rosacea, mastocytosis, cutaneous lymphomas, mycosis fungoides, and Sezary syndrome, characterized in that an effective amount of the above defined pharmaceutical dosage form is administered orally, intravenously, or topically to a subject or patient once, twice, thrice or several times daily.

### D. Dosage Forms/Ingredients

Solid pharmaceutical compositions ready for use/ingestion made from a compound of formula 3 comprise powders, granules, pellets, tablets, capsules, chewable tablets, dispersible tables, troches and lozenges. In detail:
Capsule formulations according to the invention comprise the powdery intermediate of a compound of formula 3, an intermediate blend comprising the powdery intermediate, pellets or granules obtained by conventional wet-, dry or hot-melt granulation or hot-melt extrusion or spray-drying of a suitable intermediate blend, filled in conventional capsules, e.g. hard gelatin or HPMC capsules.
The Capsule formulations from above may also comprise the powdery intermediate of a compound of formula 3 in a compacted form.
Capsule formulations according to the invention comprise the compound of formula 3 suspended or diluted in a liquid or mixture of liquids.
Tablet formulations according to the invention comprise such tablets obtained by direct compression of a suitable final blend or by tableting of pellets or granules obtained by conventional wet-, dry or hot-melt granulation or hot-melt extrusion or spray-drying of a suitable intermediate blend.

Another object of the present invention is a dosage form where a pH-adjusting or buffering agent is added for stability improvement of the active ingredient. The pH-adjusting / buffering agent may be a basic amino acid, which has an amino group and alkaline characteristics (isoelectric point, pI: 7.59-10.76), such as e.g. L-arginine, L-lysine or L-histidine. A buffering agent within the meaning of this invention is L-arginine. L-arginine has a particular suitable stabilizing effect on the compositions of this invention, e.g. by suppressing chemical degradation of compounds of formula 3.

Thus, in an embodiment, the present invention is directed to a pharmaceutical composition (e.g. an oral solid dosage form, particularly a tablet) comprising a compound of formula 3 and L-arginine for stabilizing the composition, particularly against chemical degradation; as well as one or more pharmaceutical excipients.

Suitably the pharmaceutical excipients used within this invention are conventional materials such as cellulose and its derivates, D-mannitol, corn starch, pregelatinized starch as a filler, copovidone as a binder, crospovidone as disintegrant, magnesium stearate as a lubricant, colloidal anhydrous silica as a glidant, hypromellose as a film-coating agent, polyethylene glycol as a plasticizer, titanium dioxide, iron oxide red/yellow as a pigment, and talc, etc.

In detail pharmaceutical excipients can be a first and second diluent, a binder, a disintegrant and a lubricant; an additional disintegrant and an additional glidant are a further option.
Diluents suitable for a pharmaceutical composition according to the invention are cellulose powder, microcrystalline cellulose, lactose in various crystalline modifications, dibasic calciumphosphate anhydrous, dibasic calciumphosphate dihydrate, erythritol, low substituted hydroxypropyl cellulose, mannitol, starch or modified starch (eg. pregelatinized or partially hydrolysed) or xylitol. Among those diluents mannitol and microcrystalline cellulose are employed in some instances.
Diluents that find use as the second diluent are the above mentioned diluents mannitol and microcrystalline cellulose.
Lubricants suitable for a pharmaceutical composition according to the invention are talc, polyethyleneglycol, calcium behenate, calcium stearate, sodium stearylfumarate, hydrogenated castor oil or magnesium stearate. The lubricant in some instances is magnesium stearate.
Binders suitable for a pharmaceutical composition according to the invention are copovidone (copolymerisates of vinylpyrrolidon with other vinylderivates), hydroxypropyl methylcellulose (HPMC), hydroxypropylcellulose (HPC), polyvinylpyrrolidon (povidone), pregelatinized starch, stearic-palmitic acid, low-substituted hydroxypropylcellulose (L-HPC), copovidone and pregelatinized starch being employed in some formulations. The above mentioned binders pregelatinized starch and L-HPC show additional diluent and disintegrant properties and can also be used as the second diluent or the disintegrant.
Disintegrants suitable for a pharmaceutical composition according to the present invention are corn starch, crospovidone, polacrilin potassium, croscarmellose sodium, low-substituted hydroxypropylcellulose (L-HPC) or pregelatinized starch; such as croscarmellose sodium.
As an optional glidant colloidal silicon dioxide can be used.

An exemplary composition according to the present invention comprises the diluent mannitol, microcrystalline cellulose as a diluent with additional disintegrating properties, the binder copovidone, the disintegrant croscarmellose sodium, and magnesium stearate as the lubricant.

Typical pharmaceutical compositions comprise (% by weight)

| | |
|---|---|
| 10-50% | active ingredient |
| 20-88% | diluent 1, |
| 5-50% | diluent 2, |
| 1-5% | binder, |
| 1-15% | disintegrant, and |
| 0.1-5% | lubricant. |

Pharmaceutical compositions according to some embodiments comprise (% by weight)

| | |
|---|---|
| 10-50% | active ingredient |
| 20-75% | diluent 1, |
| 5-30% | diluent 2, |
| 2-30% | binder, |
| 1-12% | disintegrant, and |
| 0.1-3% | lubricant |

Pharmaceutical compositions according to some embodiments comprise (% by weight)

| | |
|---|---|
| 10-90% | active ingredient |
| 5-70% | diluent 1, |
| 5-30% | diluent 2, |
| 0-30% | binder, |
| 1-12% | disintegrant, and |
| 0.1-3% | lubricant |

Pharmaceutical compositions according to some embodiments comprise (% by weight)

| | |
|---|---|
| 10-50% | active ingredient |
| 20-75% | diluent 1, |
| 5-30% | diluent 2, |
| 2-30% | binder, |
| 0.5-20% | buffering agent, |
| 1-12 % | disintegrant, and |
| 0.1-3% | lubricant |

Pharmaceutical compositions according to some embodiments comprise (% by weight)

| | |
|---|---|
| 30-70% | active ingredient |
| 20-75% | diluent 1, |
| 5-30% | diluent 2, |
| 2-30% | binder, |
| 0.5-20 % | buffering agent, |
| 1-12% | disintegrant, and |
| 0.1-3% | lubricant |

Pharmaceutical compositions containing 10-90% of active ingredient, such as 30-70% active ingredient (% by weight) are employed in some instances.

A tablet formulation according to the invention may be uncoated or coated, e.g. film-coated, using suitable coatings known not to negatively affect the dissolution properties of the final formulation. For instance the tablets can be provided with a seal coat for protection of the patients environment and clinical staff as well as for moisture protection purposes by dissolving a high molecular weight polymer as polyvinylpyrrolidone or hydroxypropyl-methylcellulose together with plasticizers, lubricants and optionally pigments and tensides in water or organic solvent as acetone and spraying this mixture on the tablet cores inside a coating equipment as a pan coater or a fluidized bed coater with wurster insert.

Additionally, agents such as beeswax, shellac, cellulose acetate phthalate, polyvinyl acetate phthalate, zein, film forming polymers such as hydroxypropyl cellulose, ethylcellulose and polymeric methacrylates can be applied to the tablets, provided that the coating has no substantial effect on the disintegration/dissolution of the dosage form and that the coated dosage form is not affected in its stability.

After the dosage form is film-coated, a sugar coating may be applied onto the sealed pharmaceutical dosage form. The sugar coating may comprise sucrose, dextrose, sorbitol and the like or mixtures thereof. If desired, colorants or opacifiers may be added to the sugar solution.

Solid formulations of the present invention tend to be hygroscopic. They may be packaged using PVC-blisters, PVDC-blisters or a moisture-proof packaging material such as aluminum foil blister packs, alu/alu blister, transparent or opaque polymer blister with pouch, polypropylene tubes, glass bottles and HDPE bottles optionally containing a child-resistant feature or may be tamper evident. The primary packaging material may comprise a desiccant such as molecular sieve or silica gel to improve chemical stability of the API. Opaque packaging such as colored blister materials, tubes, brown glass bottles or the like can be used to prolong shelf life of the API by reduction of photo degradation.

### E. Dosages

A dosage range of the compound of formula 3 is usually between 100 and 1000 mg, in particular between 200 and 900 mg, 300 and 900 mg or 350 and 850 mg or 390 and 810 mg. It is possible to give one or two tablets, where in some instances two tablets for a daily oral dosage of 100, 200, 300, 350, 400, 450, 500, 550, 600, 650, 700, 750, 800, 850, 900 mg, and in some instances 350, 400, 450, 750, 800, 850 are employed.

The dosages range can be achieved by one tablet or by two tablets; in some instances two tablets are administered, each containing half of the dosage.

The application of the active ingredient may occur up to three times a day, such as one or two times a day. Particular dosage strengths are 400 mg or 800 mg.

### F. Used Terms and Definitions

Terms not specifically defined herein should be given the meanings that would be given to them by one of skill in the art in light of the disclosure and the context. As used in the specification, however, unless specified to the contrary, the following terms have the meaning indicated and the following conventions are adhered to.

The term, “about” means 5% more or less of the specified value. Thus, about 100 minutes could also be read as from 95 to 105 minutes.

In case a compound of the present invention is depicted in form of a chemical name and as a formula in case of any discrepancy the formula shall prevail. An asterisk is may be used in sub-formulas to indicate the bond which is connected to the core molecule as defined.

Unless specifically indicated, throughout the specification and the appended claims, a given chemical formula or name shall encompass tautomers and all stereo, optical and geometrical isomers (e.g. enantiomers, diastereomers, E/Z isomers etc...) and racemates thereof as well as mixtures in different proportions of the separate enantiomers, mixtures of diastereomers, or mixtures of any of the foregoing forms where such isomers and enantiomers exist, as well as salts, including pharmaceutically acceptable salts thereof and solvates thereof such as for instance hydrates including solvates of the free compounds or solvates of a salt of the compound.

The term “substituted” as used herein, means that any one or more hydrogens on the designated atom is replaced with a selection from the indicated group, provided that the designated atom’s normal valence is not exceeded, and that the substitution results in a stable compound.

By the term “optionally substituted” is meant within the scope of the invention the above-mentioned group, optionally substituted by a lower-molecular group. Examples of lower-molecular groups regarded as chemically meaningful are groups consisting of 1-200 atoms. Of interest are such groups that have no negative effect on the pharmacological efficacy of the compounds. For example the groups may comprise:
Straight-chain or branched carbon chains, optionally interrupted by heteroatoms, optionally substituted by rings, heteroatoms or other common functional groups.
Aromatic or non-aromatic ring systems consisting of carbon atoms and optionally heteroatoms, which may in turn be substituted by functional groups.
A number of aromatic or non-aromatic ring systems consisting of carbon atoms and optionally heteroatoms which may be linked by one or more carbon chains, optionally interrupted by heteroatoms, optionally substituted by heteroatoms or other common functional groups.

The compounds disclosed herein can exist as therapeutically acceptable salts. The present invention includes compounds listed above in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley- VCHA, Zurich, Switzerland, 2002).

The term “therapeutically acceptable salt,” as used herein, represents salts or zwitterionic forms of the compounds disclosed herein which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L- ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, Succinate, Sulfonate, tartrate, L-tartrate, trichloroacetate, trifluoroacetate, phosphate, glutamate, bicarbonate, para- toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl Sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds disclosed herein, and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N, N-dibenzylphenethylamine, 1-ephenamine, and N,P-dibenzylethylenediamine. Other representative organic amines useful for the formation of base addition salts include ethylenediamine, ethanolamine, diethanolamine, piperidine, and piperazine.

While it may be possible for the compounds of the subject invention to be administered as the raw chemical, it is also possible to present them as a pharmaceutical formulation. Accordingly, provided herein are pharmaceutical formulations which comprise one or more of certain compounds disclosed herein, or one or more pharmaceutically acceptable salts, esters, prodrugs, amides, or solvates thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art; e.g., in Remington’s Pharmaceutical Sciences. The pharmaceutical compositions disclosed herein may be manufactured in any manner known in the art, e.g., by means of conventional mixing, dissolving, granulating, dragee-making, levigating, emulsifying, encapsulating, entrapping or compression processes.

“Heterocyclic rings” (“het”) include five-, six- or seven-membered, saturated or unsaturated heterocyclic rings or 5-10 membered, bicyclic hetero rings which may contain one, two or three heteroatoms, selected from among oxygen, sulphur and nitrogen; the ring may be linked to the molecule by a carbon atom or, if present, by a nitrogen atom. The following are examples of five-, six- or seven-membered, saturated or unsaturated heterocyclic rings:

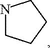

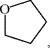

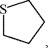

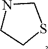

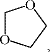

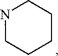

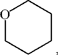

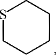

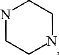

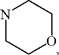

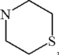

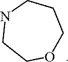

Unless stated otherwise, a heterocyclic ring may be provided with a keto group. Examples include:

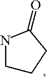

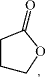

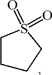

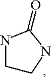

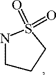

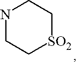

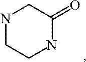

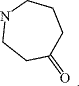

Examples of 5-10-membered bicyclic hetero rings are pyrrolizine, indole, indolizine, isoindole, indazole, purine, quinoline, isoquinoline, benzimidazole, benzofurane, benzopyrane, benzothiazole, benzoisothiazole, pyridopyrimidine, pteridine, pyrimidopyrimidine,

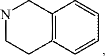

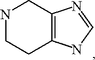

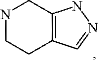

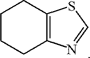

Although the term heterocyclic ring includes heterocyclic aromatic groups, the term heterocyclic aromatic groups (“hetaryl”) denotes five- or six-membered heterocyclic aromatic groups or 5-10 membered, bicyclic hetaryl rings which may contain one, two or three heteroatoms, selected from among oxygen, sulphur and nitrogen, which contain sufficient conjugated double bonds that an aromatic system is formed. The ring may be linked to the molecule through a carbon atom or if present through a nitrogen atom. The following are examples of five- or six-membered heterocyclic aromatic groups:

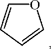

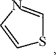

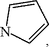

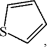

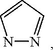

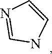

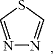

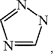

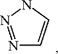

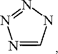

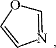

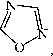

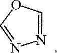

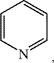

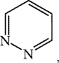

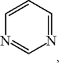

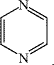

Examples of 5-10-membered bicyclic hetaryl rings include pyrrolizine, indole, indolizine, isoindole, indazole, purine, quinoline, isoquinoline, benzimidazole, benzofuran, benzopyrane, benzothiazole, benzoisothiazole, pyridopyrimidine, pteridine, pyrimidopyrimidine.

The term “halogen” as used herein means a halogen substituent selected from fluoro, chloro, bromo or iodo.

By the term “C₁₋₆-alkyl” (including those which are part of other groups) are meant branched and unbranched alkyl groups with 1 to 6 carbon atoms, and by the term “C₁₋₄ -alkyl” are meant branched and unbranched alkyl groups with 1 to 4 carbon atoms. Alkyl groups with 1 to 4 carbon atoms are present in some instances. Examples of these include: methyl, ethyl, n-propyl, iso-propyl, n-butyl, iso-butyl, sec-butyl, tert-butyl, n-pentyl, iso-pentyl, neo-pentyl or hexyl. The abbreviations Me, Et, n-Pr, i-Pr, n-Bu, i-Bu, t-Bu, etc. may optionally also be used for the above-mentioned groups. Unless stated otherwise, the definitions propyl, butyl, pentyl and hexyl include all the possible isomeric forms of the groups in question. Thus, for example, propyl includes n-propyl and iso-propyl, butyl includes iso-butyl, sec-butyl and tert-butyl etc.

By the term “C₁₋₆-alkylene” (including those which are part of other groups) are meant branched and unbranched alkylene groups with 1 to 6 carbon atoms and by the term “C₁₋₄-alkylene” are meant branched and unbranched alkylene groups with 1 to 4 carbon atoms. Alkylene groups with 1 to 4 carbon atoms are present in some instances. Examples include: methylene, ethylene, propylene, 1-methylethylene, butylene, 1-methylpropylene, 1,1-dimethylethylene, 1,2-dimethylethylene, pentylene, 1,1-dimethylpropylene, 2,2-dimethylpropylene, 1,2-dimethylpropylene, 1,3-dimethylpropylene or hexylene. Unless stated otherwise, the definitions propylene, butylene, pentylene and hexylene also include all the possible isomeric forms of the relevant groups with the same number of carbons. Thus for example propyl also includes 1-methylethylene and butylene includes 1-methylpropylene, 1,1-dimethylethylene, 1,2-dimethylethylene.

The term “ C₂₋₆-alkenyl ” (including those which are part of other groups) denotes branched and unbranched alkenyl groups with 2 to 6 carbon atoms and the term “C₂₋₄-alkenyl” denotes branched and unbranched alkenyl groups with 2 to 4 carbon atoms, provided that they have at least one double bond. Employed in some instances are alkenyl groups with 2 to 4 carbon atoms. Examples include: ethenyl or vinyl, propenyl, butenyl, pentenyl, or hexenyl. Unless otherwise stated, the definitions propenyl, butenyl, pentenyl and hexenyl include all possible isomeric forms of the groups in question. Thus, for example, propenyl includes 1-propenyl and 2-propenyl, butenyl includes 1-, 2- and 3-butenyl, 1-methyl-1-propenyl, 1-methyl-2-propenyl etc.

By the term “C₂₋₆-alkenylene” (including those which are part of other groups) are meant branched and unbranched alkenylene groups with 2 to 6 carbon atoms and by the term “C₂₋₄-alkenylene” are meant branched and unbranched alkylene groups with 2 to 4 carbon atoms. Alkenylene groups with 2 to 4 carbon atoms are present in some instances. Examples include: ethenylene, propenylene, 1-methylethenylene, butenylene, 1-methylpropenylene, 1,1-dimethylethenylene, 1,2-dimethylethenylene, pentenylene, 1,1-dimethylpropenylene, 2,2-dimethylpropenylene, 1,2-dimethylpropenylene, 1,3-dimethylpropenylene or hexenylene. Unless stated otherwise, the definitions propenylene, butenylene, pentenylene and hexenylene include all the possible isomeric forms of the respective groups with the same number of carbons. Thus, for example, propenyl also includes 1-methylethenylene and butenylene includes 1-methylpropenylene, 1,1-dimethylethenylene, 1,2-dimethylethenylene.

By the term “C₂₋₆-alkynyl” (including those which are part of other groups) are meant branched and unbranched alkynyl groups with 2 to 6 carbon atoms and by the term “C₂₋₄-alkynyl” are meant branched and unbranched alkynyl groups with 2 to 4 carbon atoms, provided that they have at least one triple bond. Alkynyl groups with 2 to 4 carbon atoms are present in some instances. Examples include: ethynyl, propynyl, butynyl, pentynyl, or hexynyl. Unless stated otherwise, the definitions propynyl, butynyl, pentynyl and hexynyl include all the possible isomeric forms of the respective groups. Thus, for example, propynyl includes 1-propynyl and 2-propynyl, butynyl includes 1-, 2- and 3-butynyl, 1-methyl-1-propynyl, 1-methyl-2-propynyl etc.

By the term “C₂₋₆-alkynylene” (including those which are part of other groups) are meant branched and unbranched alkynylene groups with 2 to 6 carbon atoms and by the term “C₂₋₄-alkynylene” are meant branched and unbranched alkylene groups with 2 to 4 carbon atoms. Alkynylene groups with 2 to 4 carbon atoms are present in some instances. Examples include: ethynylene, propynylene, 1-methylethynylene, butynylene, 1-methylpropynylene, 1,1-dimethylethynylene, 1,2-dimethylethynylene, pentynylene, 1,1-dimethylpropynylene, 2,2-dimethylpropynylene, 1,2-dimethylpropynylene, 1,3-dimethylpropynylene or hexynylene. Unless stated otherwise, the definitions propynylene, butynylene, pentynylene and hexynylene include all the possible isomeric forms of the respective groups with the same number of carbons. Thus for example propynyl also includes 1-methylethynylene and butynylene includes 1-methylpropynylene, 1,1-dimethylethynylene, 1,2-dimethylethynylene.

The term “C₃₋₆-cycloalkyl” (including those which are part of other groups) as used herein means cyclic alkyl groups with 3 to 8 carbon atoms, where in some instances such groups are cyclic alkyl groups with 5 to 6 carbon atoms. Examples include: cyclopropyl, cyclobutyl, cyclopentyl or cyclohexyl.

By the term “C₁₋₆-haloalkyl” (including those which are part of other groups) are meant branched and unbranched alkyl groups with 1 to 6 carbon atoms wherein one or more hydrogen atoms are replaced by a halogen atom selected from among fluorine, chlorine or bromine, such as fluorine and chlorine, e.g., fluorine. By the term “C₁₋₄-haloalkyl” are meant correspondingly branched and unbranched alkyl groups with 1 to 4 carbon atoms, wherein one or more hydrogen atoms are replaced analogously to what was stated above. C₁₋₄-haloalkyl is presentin some instances. Examples include: CH₂F, CHF₂, CF₃.

The term “C₁₋ₙ-alkyl”, wherein n is an integer from 2 to n, either alone or in combination with another radical denotes an acyclic, saturated, branched or linear hydrocarbon radical with 1 to n C atoms. For example the term C₁₋₅-alkyl embraces the radicals H₃C—, H₃C—CH₂—, H₃C—CH₂—CH₂—, H₃C—CH(CH₃)—, H₃C—CH₂—CH₂—CH₂—, H₃C—CH₂—CH(CH₃)—, H₃C—CH(CH₃)—CH₂—, H₃C—C(CH₃)₂—, H₃C—CH₂—CH₂—CH₂—CH₂—, H₃C—CH₂—CH₂—CH(CH₃)—, H₃C—CH₂—CH(CH₃)—CH₂—, H₃C—CH(CH₃)—CH₂—CH₂—, H₃C—CH₂—C(CH₃)₂—, H₃C—C(CH₃)₂—CH₂—, H₃C—CH(CH₃)—CH(CH₃)— and H₃C—CH₂—CH(CH₂CH₃)—.

The term “C₁₋ₙ-haloalkyl”, wherein n is an integer from 2 to n, either alone or in combination with another radical denotes an acyclic, saturated, branched or linear hydrocarbon radical with 1 to n C atoms wherein one or more hydrogen atoms are replaced by a halogen atom selected from among fluorine, chlorine or bromine, such as fluorine and chlorine, e.g., fluorine. Examples include: CH₂F, CHF₂, CF₃.

The term “C₁₋ₙ-alkylene” wherein n is an integer 2 to n, either alone or in combination with another radical, denotes an acyclic, straight or branched chain divalent alkyl radical containing from 1 to n carbon atoms. For example the term C₁₋₄-alkylene includes —CH₂—, —CH₂—CH₂—, —CH(CH₃)—, —CH₂—CH₂—CH₂—, —C(CH₃)₂—, —CH(CH₂CH₃)—, —CH(CH₃)—CH₂—, —CH₂—CH(CH₃)—, —CH₂—CH₂—CH₂—CH₂—, —CH₂—CH₂—CH(CH₃)—, —CH(CH₃)—CH₂—CH₂—, —CH₂—CH(CH₃)—CH₂—, —CH₂—C(CH₃)₂—, —C(CH₃)₂—CH₂—, —CH(CH₃)—CH(CH ₃)—, —CH₂—CH(CH₂CH₃)—, —CH(CH₂CH₃)—CH₂—, —CH(CH₂CH₂CH₃)—, —CH(CH(CH₃))₂— and —C(C H₃)(CH₂CH₃)—.

The term “C₂₋ₙ-alkenyl”, is used for a group as defined in the definition for “C₁₋ₙ-alkyl” with at least two carbon atoms, if at least two of those carbon atoms of said group are bonded to each other by a double bond.

The term “C₂₋ₙ-alkynyl”, is used for a group as defined in the definition for “C₁₋ₙ-alkyl” with at least two carbon atoms, if at least two of those carbon atoms of said group are bonded to each other by a triple bond.

The term “C₃₋ₙ-cycloalkyl”, wherein n is an integer from 4 to n, either alone or in combination with another radical denotes a cyclic, saturated, unbranched hydrocarbon radical with 3 to n C atoms. For example the term C₃₋₇-cycloalkyl includes cyclopropyl, cyclobutyl, cyclopentyl, cyclohexyl and cycloheptyl.

It must be noted that as used herein and in the appended claims, the singular forms “a,” “an,” and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those having skill in the art, and so forth.

By “an individual suffering from or at risk of suffering from an aging-associated cognitive impairment” is meant an individual that is about more than 50% through its expected lifespan, such as more than 60%, e.g., more than 70%, such as more than 75%, 80%, 85%, 90%, 95% or even 99% through its expected lifespan. The age of the individual will depend on the species in question. Thus, this percentage is based on the predicted life-expectancy of the species in question. For example, in humans, such an individual is 50 year old or older, e.g., 60 years old or older, 70 years old or older, 80 years old or older, 90 years old or older, and usually no older than 100 years old, such as 90 years old., i.e., between the ages of about 50 and 100, e.g., 50 ... 55 ... 60 ... 65 ... 70 ... 75 ... 80 ... 85 ... 90 ... 95 ... 100 years old or older, or any age between 50 - 1000, that suffers from an aging-associated condition as further described below, e.g., cognitive impairment associated with the natural aging process; an individual that is about 50 years old or older, e.g., 60 years old or older, 70 years old or older, 80 years old or older, 90 years old or older, and usually no older than 100 years old, i.e., between the ages of about 50 and 100, e.g., 50 ... 55 ... 60 ... 65 ... 70 ... 75 ... 80 ... 85 ... 90 ... 95 ... 100 years old, that has not yet begun to show symptoms of an aging-associated condition e.g., cognitive impairment; an individual of any age that is suffering from a cognitive impairment due to an aging-associated disease, as described further below, and an individual of any age that has been diagnosed with an aging-associated disease that is typically accompanied by cognitive impairment, where the individual has not yet begun to show symptoms of cognitive impairment. The corresponding ages for non-human subjects are known and are intended to apply herein.

As summarized elsewhere, in some instances the subject is a mammal. Mammalian species that may be treated with the present methods include canines and felines; equines; bovines; ovines; etc., and primates, including humans. The subject methods, compositions, and reagents may also be applied to animal models, including small mammals, e.g., murine, lagomorpha, etc., for example, in experimental investigations.

As used herein and as described above, “treatment” refers to any of (i) the prevention of the disease or disorder, or (ii) the reduction or elimination of symptoms of the disease or disorder. Treatment may be effected prophylactically (prior to the onset of disease) or therapeutically (following the onset of the disease). The effect may be prophylactic in terms of completely or partially preventing a disease or symptom thereof and/or may be therapeutic in terms of a partial or complete cure for a disease and/or adverse effect attributable to the disease. Thus, the term “treatment” as used herein covers any treatment of an aging-related disease or disorder in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. Treatment may result in a variety of different physical manifestations, e.g., modulation in gene expression, rejuvenation of tissue or organs, etc. The therapeutic agent may be administered before, during or after the onset of disease. The treatment of ongoing disease, where the treatment stabilizes or reduces the undesirable clinical symptoms of the patient, is of particular interest. Such treatment may be performed prior to complete loss of function in the affected tissues. The subject therapy may be administered during the symptomatic stage of the disease, and in some cases after the symptomatic stage of the disease.

### G. Combinations

The compounds of general formula 1 may be used on their own or combined with other active substances of formula 1 according to the invention. The compounds of general formula 1 may optionally also be combined with other pharmacologically active substances. These include, β2-adrenoceptor-agonists (short and long-acting), anti-cholinergics (short and long-acting), anti-inflammatory steroids (oral and topical corticosteroids), cromoglycate, methylxanthine, dissociated-glucocorticoidmimetics, PDE3 inhibitors, PDE4- inhibitors, PDE7- inhibitors, LTD4 antagonists, EGFR- inhibitors, Dopamine agonists, PAF antagonists, Lipoxin A4 derivatives, FPRL1 modulators, LTB4-receptor (BLT1, BLT2) antagonists, Histamine H1 receptor antagonists, Histamine H4 receptor antagonists, dual Histamine H1/H3-receptor antagonists, PI3-kinase inhibitors, inhibitors of non-receptor tyrosine kinases as for example LYN, LCK, SYK, ZAP-70, FYN, BTK or ITK, inhibitors of MAP kinases as for example p38, ERK1, ERK2, JNK1, JNK2, JNK3 or SAP, inhibitors of the NF-κB signaling pathway as for example IKK2 kinase inhibitors, iNOS inhibitors, MRP4 inhibitors, leukotriene biosynthese inhibitors as for example 5-Lipoxygenase (5-LO) inhibitors, cPLA2 inhibitors, Leukotriene A4 Hydrolase inhibitors or FLAP inhibitors, Non-steroidal anti-inflammatory agents (NSAIDs), CRTH2 antagonists, DP1-receptor modulators, Thromboxane receptor antagonists, additional CCR3 antagonists, CCR4 antagonists, CCR1 antagonists, CCR5 antagonists, CCR6 antagonists, CCR7 antagonists, CCR8 antagonists, CCR9 antagonists, CCR30 antagonists, CXCR3 antagonists, CXCR4 antagonists, CXCR² antagonists, CXCR1 antagonists, CXCR5 antagonists, CXCR6 antagonists, CX3CR3 antagonists, Neurokinin (NK1, NK2) antagonists, Sphingosine 1-Phosphate receptor modulators, Sphingosine 1 phosphate lyase inhibitors, Adenosine receptor modulators as for example A2a-agonists, modulators of purinergic receptors as for example P2X7 inhibitors, Histone Deacetylase (HDAC) activators, Bradykinin (BK1, BK2) antagonists, TACE inhibitors, PPAR gamma modulators, Rho-kinase inhibitors, interleukin 1-beta converting enzyme (ICE) inhibitors, Toll-Like receptor (TLR) modulators, HMG-CoA reductase inhibitors, VLA-4 antagonists, ICAM-1 inhibitors, SHIP agonists, GABAa receptor antagonist, ENaC-inhibitors, Melanocortin receptor (MC1R, MC2R, MC3R, MC4R, MC5R) modulators, CGRP antagonists, Endothelin antagonists, TNFα antagonists, anti-TNF antibodies, anti-GM-CSF antibodies, anti-CD46 antibodies, anti-IL-1 antibodies, anti-IL-2 antibodies, anti-IL-4 antibodies, anti-IL-5 antibodies, anti-IL-13 antibodies, anti-IL-4/IL-13 antibodies, anti-TSLP antibodies, anti-OX40 antibodies, mucoregulators, immunotherapeutic agents, compounds against swelling of the airways, compounds against cough, VEGF inhibitors, but also combinations of two or three active substances.

In some embodiments, the other active substances are betamimetics, anticholinergics, corticosteroids, PDE4-inhibitors, LTD4-antagonists, EGFR-inhibitors, CRTH2 inhibitors, 5-LO-inhibitors, Histamine receptor antagonists and SYK-inhibitors, but also combinations of two or three active substances, i.e.:
Betamimetics with corticosteroids, PDE4-inhibitors, CRTH2-inhibitors or LTD4-antagonists,
Anticholinergics with betamimetics, corticosteroids, PDE4-inhibitors, CRTH2-inhibitors or LTD4-antagonists,
Corticosteroids with PDE4-inhibitors, CRTH2-inhibitors or LTD4-antagonists
PDE4-inhibitors with CRTH2-inhibitors or LTD4-antagonists
CRTH2-inhibitors with LTD4-antagonists.
In these embodiments, the compounds that make up the combination are co-administered to a subject. The terms “co-administration” and “in combination with” include the administration of two or more therapeutic agents either simultaneously, concurrently or sequentially within no specific time limits. In one embodiment, the agents are present in the cell or in the subject’s body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent. “Concomitant administration” of a known therapeutic drug with a pharmaceutical composition of the present disclosure means administration of the compound and second agent at such time that both the known drug and the composition of the present invention will have a therapeutic effect. Such concomitant administration may involve concurrent (i.e. at the same time), prior, or subsequent administration of the drug with respect to the administration of a subject compound. Routes of administration of the two agents may vary, where representative routes of administrationare described in greater detail below. A person of ordinary skill in the art would have no difficulty determining the appropriate timing, sequence and dosages of administration for particular drugs and compounds of the present disclosure. In some embodiments, the compounds (e.g., a subject compound and the at least one additional compound) are administered to the subject within twenty-four hours of each other, such as within 12 hours of each other, within 6 hours of each other, within 3 hours of each other, or within 1 hour of each other. In certain embodiments, the compounds are administered within 1 hour of each other. In certain embodiments, the compounds are administered substantially simultaneously. By administered substantially simultaneously is meant that the compounds are administered to the subject within about 10 minutes or less of each other, such as 5 minutes or less, or 1 minute or less of each other.

### H. Pharmaceutical Forms

Suitable preparations for administering the compounds of formula 1 and the co-crystal or salt forms of formulae 2 and 2a include for example tablets, capsules, suppositories, solutions and powders etc. The content of the pharmaceutically active compound(s) should be in the range from 0.05 to 90 wt.-%, such as 0.1 to 50 wt.-% of the composition as a whole. Suitable tablets may be obtained, for example, by mixing the active substance(s) with known excipients, for example inert diluents such as calcium carbonate, calcium phosphate or lactose, disintegrants such as corn starch or alginic acid, binders such as starch or gelatin, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly, the tablet coating may consist of a number or layers to achieve delayed release, possibly using the excipients mentioned above for the tablets.

Syrups or elixirs containing the active substances or combinations thereof according to the invention may additionally contain a sweetener such as saccharine, cyclamate, glycerol or sugar and a flavor enhancer, e.g. a flavoring such as vanillin or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Solutions are prepared in the usual way, e.g. with the addition of isotonic agents, preservatives such as p-hydroxybenzoates or stabilizers such as alkali metal salts of ethylenediaminetetraacetic acid, optionally using emulsifiers and/or dispersants, while if water is used as diluent, for example, organic solvents may optionally be used as solubilizers or dissolving aids, and the solutions may be transferred into injection vials or ampoules or infusion bottles.

Capsules containing one or more active substances or combinations of active substances may for example be prepared by mixing the active substances with inert carriers such as lactose or sorbitol and packing them into gelatin capsules.

Suitable suppositories may be made for example by mixing with carriers provided for this purpose, such as neutral fats or polyethyleneglycol or the derivatives thereof.

Excipients which may be used include, for example, water, pharmaceutically acceptable organic solvents such as paraffins (e.g. petroleum fractions), vegetable oils (e.g. groundnut or sesame oil), mono- or polyfunctional alcohols (e.g. ethanol or glycerol), carriers such as e.g. natural mineral powders (e.g. kaolins, clays, talc, chalk), synthetic mineral powders (e.g. highly dispersed silicic acid and silicates), sugars (e.g. cane sugar, lactose and glucose), emulsifiers (e.g. lignin, spent sulphite liquors, methylcellulose, starch and polyvinylpyrrolidone) and lubricants (e.g. magnesium stearate, talc, stearic acid and sodium lauryl sulphate).

For oral use the tablets may obviously contain, in addition to the carriers specified, additives such as sodium citrate, calcium carbonate and dicalcium phosphate together with various additional substances such as starch, e.g., potato starch, gelatin and the like. Lubricants such as magnesium stearate, sodium laurylsulphate and talc may also be used to produce the tablets. In the case of aqueous suspensions, the active substances may be combined with various flavor enhancers or colorings in addition to the abovementioned excipients.

For administering the compounds of formula 1 or the co-crystal or salt forms of formulae 2 and 2a preparations or pharmaceutical formulations which are suitable for inhalation may be employed. Inhalable preparations include inhalable powders, propellant-containing metered-dose aerosols or propellant-free inhalable solutions. Within the scope of the present invention, the term propellant-free inhalable solutions also include concentrates or sterile inhalable solutions ready for use. The formulations which may be used within the scope of the present invention are described in more detail in the next part of the specification.

The inhalable powders which may be used according to the invention may contain a compound of formula 1 or a co-crystal or salt form of formulae 2 and 2a either on their own or in admixture with suitable physiologically acceptable excipients.

If the active substances of the compounds of formula 1 or the co-crystal or salt forms of formulae 2 and 2a are present in admixture with physiologically acceptable excipients, the following physiologically acceptable excipients may be used to prepare these inhalable powders according to the invention: monosaccharides (e.g. glucose or arabinose), disaccharides (e.g. lactose, saccharose, maltose), oligo- and polysaccharides (e.g. dextrans), polyalcohols (e.g. sorbitol, mannitol, xylitol), salts (e.g. sodium chloride, calcium carbonate) or mixtures of these excipients. In some instances, mono- or disaccharides are used, such as lactose or glucose, e.g., in the form of their hydrates, e.g., lactose, such as lactose monohydrate.

Within the scope of the inhalable powders according to the invention the excipients have a maximum average particle size of up to 250 µm, such as between 10 and 150 µm, and including between 15 and 80 µm. It may sometimes seem appropriate to add finer excipient fractions with an average particle size of 1 to 9 µm to the excipient mentioned above. These finer excipients are also selected from the group of possible excipients listed hereinbefore. Finally, in order to prepare the inhalable powders according to the invention, micronized active substance of the compounds of formula 1 or the co-crystal or salt forms of formulae 2 and 2a, such as with an average particle size of 0.5 to 10 µm, including from 1 to 5 µm, is added to the excipient mixture. Processes for producing the inhalable powders according to the invention by grinding and micronizing and finally mixing the ingredients together are known from the prior art.

The inhalable powders according to the invention may be administered using inhalers known from the prior art.

The inhalation aerosols containing propellant gas according to the invention may contain a compound of formula 1 or a co-crystal or salt form of formulae 2 and 2a dissolved in the propellant gas or in dispersed form. The compounds of formula 1 or the co-crystal or salt forms of formulae 2 and 2a may be contained in separate formulations or in a common formulation, in which they are either both dissolved, both dispersed or in each case only one component is dissolved and the other is dispersed. The propellant gases which may be used to prepare the inhalation aerosols are known from the prior art. Suitable propellant gases are selected from among hydrocarbons such as n-propane, n-butane or isobutane and halohydrocarbons such as fluorinated derivatives of methane, ethane, propane, butane, cyclopropane or cyclobutane. The abovementioned propellant gases may be used on their own or mixed together. In some instances, propellant gases are halogenated alkane derivatives selected from TG134a and TG227 and mixtures thereof.

The propellant-driven inhalation aerosols may also contain other ingredients such as co-solvents, stabilizers, surfactants, antioxidants, lubricants and pH adjusters. All these ingredients are known in the art.

The propellant-driven inhalation aerosols according to the invention mentioned above may be administered using inhalers known in the art (MDIs = metered dose inhalers). Moreover, the active substances of the compounds of formula 1 or the co-crystal or salt forms of formulae 2 and 2a according to the invention may be administered in the form of propellant-free inhalable solutions and suspensions. The solvent used may be an aqueous or alcoholic, such as an ethanolic solution. The solvent may be water on its own or a mixture of water and ethanol. The relative proportion of ethanol compared with water is not limited but the maximum is in some instances up to 70 percent by volume, such as up to 60 percent by volume and including up to 30 percent by volume. The remainder of the volume is made up of water. The solutions or suspensions containing a compound of formula 1 or a co-crystal or salt form of formulae 2 and 2a are adjusted to a pH of 2 to 7, such as 2 to 5, using suitable acids. The pH may be adjusted using acids selected from inorganic or organic acids. Examples of particularly suitable inorganic acids include hydrochloric acid, hydrobromic acid, nitric acid, sulphuric acid and/or phosphoric acid. Examples of particularly suitable organic acids include ascorbic acid, citric acid, malic acid, tartaric acid, maleic acid, succinic acid, fumaric acid, acetic acid, formic acid and/or propionic acid etc. In some instances, the inorganic acids are hydrochloric and sulphuric acids. It is also possible to use the acids which have already formed an acid addition salt with one of the active substances. Of the organic acids, ascorbic acid, fumaric acid and citric acid are employed in some instances. If desired, mixtures of the above acids may be used, particularly in the case of acids which have other properties in addition to their acidifying qualities, e.g. as flavourings, antioxidants or complexing agents, such as citric acid or ascorbic acid, for example. According to the invention, in some instances hydrochloric acid is employed to adjust the pH.

If desired, the addition of editic acid (EDTA) or one of the known salts thereof, sodium edetate, as stabilizer or complexing agent may be omitted in these formulations. Other embodiments may contain this compound or these compounds. In an embodiment the content based on sodium edetate is less than 100 mg/100ml, such as less than 50 mg/100ml, and including less than 20 mg/100ml. Inhalable solutions in which the content of sodium edetate is from 0 to 10 mg/100ml are employed in some instances. Co-solvents and/or other excipients may be added to the propellant-free inhalable solutions, such as those which contain hydroxyl groups or other polar groups, e.g. alcohols - particularly isopropyl alcohol, glycols - particularly propyleneglycol, polyethyleneglycol, polypropyleneglycol, glycolether, glycerol, polyoxyethylene alcohols and polyoxyethylene fatty acid esters. The terms excipients and additives in this context denote any pharmacologically acceptable substance which is not an active substance but which can be formulated with the active substance or substances in the physiologically suitable solvent in order to improve the qualitative properties of the active substance formulation. In some embodiments, these substances have no pharmacological effect or, in connection with the desired therapy, no appreciable or at least no undesirable pharmacological effect. The excipients and additives include, for example, surfactants such as soya lecithin, oleic acid, sorbitan esters, such as polysorbates, polyvinylpyrrolidone, other stabilizers, complexing agents, antioxidants and/or preservatives which guarantee or prolong the shelf life of the finished pharmaceutical formulation, flavourings, vitamins and/or other additives known in the art. The additives also include pharmacologically acceptable salts such as sodium chloride as isotonic agents.

In some embodiments, excipients include antioxidants such as ascorbic acid, for example, provided that it has not already been used to adjust the pH, vitamin A, vitamin E, tocopherols and similar vitamins and provitamins occurring in the human body.

Preservatives may be used to protect the formulation from contamination with pathogens. Suitable preservatives are those which are known in the art, particularly cetyl pyridinium chloride, benzalkonium chloride or benzoic acid or benzoates such as sodium benzoate in the concentration known from the prior art. The preservatives mentioned above may be present in concentrations of up to 50 mg/100 ml, such as between 5 and 20 mg/100 ml.

In some embodiments, the formulations contain, in addition to the solvent water and the compounds of formula 1 or the co-crystal or salt forms of formulae 2 and 2a, only benzalkonium chloride and sodium edetate. In an embodiment, no sodium edetate is present.

The dosage of the compounds according to the invention is naturally highly dependent on the method of administration and the complaint which is being treated. When administered by inhalation the compounds of formula 1 or the co-crystal or salt forms of formulae 2 and 2a are characterized by a high potency even at doses in the µg range. The compounds of formula 1 or the co-crystal or salt forms of formulae 2 and 2a may also be used effectively above the µg range. The dosage may then be in the gram range, for example.

In another aspect the present invention relates to the above-mentioned pharmaceutical formulations as such which are characterized in that they contain a compound of formula 1 or a co-crystal or salt form of formulae 2 and 2a, particularly the above-mentioned pharmaceutical formulations which can be administered by inhalation.

The following examples of formulations illustrate the present invention without restricting its scope:

### I. Examples of Pharmaceutical Formulations

A)

| | |
|---|---|
| Tablets | per tablet |
| active substance 1, 2, or 2a | 100 mg |
| lactose | 140 mg |
| maize starch | 240 mg |
| polyvinylpyrrolidone | 15 mg |
| magnesium stearate | 5 mg |
| | 500 mg |

The finely ground active substance, lactose and some of the maize starch are mixed together. The mixture is screened, then moistened with a solution of polyvinylpyrrolidone in water, kneaded, wet granulated and dried. The granules, the remaining maize starch and the magnesium stearate are screened and mixed together. The mixture is pressed into tablets of suitable shape and size.

B)

| | |
|---|---|
| Tablets | per tablet |
| active substance 1, 2, or 2a | 80 mg |
| lactose | 55 mg |
| maize starch | 190 mg |
| microcrystalline cellulose | 35 mg |
| polyvinylpyrrolidone | 15 mg |
| sodium carboxymethyl starch | 23 mg |
| magnesium stearate | 2 mg |
| | 400 mg |

The finely ground active substance, some of the corn starch, lactose, microcrystalline cellulose and polyvinylpyrrolidone are mixed together, the mixture is screened and worked with the remaining corn starch and water to form a granulate which is dried and screened. The sodium carboxymethyl starch and the magnesium stearate are added and mixed in and the mixture is compressed to form tablets of a suitable size.

C)

| | |
|---|---|
| Ampoule solution | |
| active substance 1, 2, or 2a | 50 mg |
| sodium chloride | 50 mg |
| water for inj. | 5 ml |

The active substance is dissolved in water at its own pH or optionally at pH 5.5 to 6.5 and sodium chloride is added to make the solution isotonic. The resulting solution is filtered to remove pyrogens and the filtrate is transferred under aseptic conditions into ampoules which are then sterilized and heat-sealed. The ampoules contain 5 mg, 25 mg and 50 mg of active substance.

D)

| | |
|---|---|
| Metering aerosol | |
| active substance 1, 2, or 2a | 0.005 |
| sorbitan trioleate | 0.1 |
| monofluorotrichloromethane and | |
| TG134a : TG227 2:1 | ad 100 |

The suspension is transferred into a conventional aerosol container with metering valve. Preferably 50 µl suspension are released on each actuation. The active substance may also be released in higher doses if desired (e.g. 0.02 wt.-%).

E)

| | |
|---|---|
| Solutions (in mg/100ml) | |
| active substance 1, 2, or 2a | 333.3 mg |
| benzalkonium chloride | 10.0 mg |
| EDTA | 50.0 mg |
| HCl (1N) | ad pH 2.4 |

This solution can be prepared in the usual way.

F)

| | |
|---|---|
| Inhalable powder | |
| active substance 1, 2, or 2a | 12 µg |
| lactose monohydrate | ad 25 mg |

The inhalable powder is prepared in the usual way by mixing the individual ingredients.

### J. Topical Formulations

Suitable preparations for administering the compounds of formula 1 and the co-crystal or salt forms of formulae 2 and 2a may also include topical formulations. Administration of polar, hydrophilic molecules as exhibited by the compounds of formula 1 and the co-crystal or salt forms of formulae 2 and 2a is well-known in the art. For example, powdered forms may simply be mixed with dimethylsulfoxide (DMSO) in a therapeutic amount, and administered topically to the affected area. In other embodiments, the compounds of formula 1 and the co-crystal or salt forms of formulae 2 and 2a may be prepared as a topical formulation according to the methods of, by way of example and not limitation, U.S. Pat. Application No. US20170266289, U.S. Pat. Application No. 2016/0058725, which are both incorporated by reference herein in their entirety.

### K. Indications

The methods of the invention further comprise treating skin symptoms and disorders, including by way of example and not limitation, pruritis (itching) or xerosis (dry or scaly skin). The methods further comprise treating the root causes of skin disorders through inhibition of the eotaxin/CCR3 pathway by administering the compositions of the invention including Compound 1 and its analogues described herein.

### Pruritis

Pruritis is the subjective sensation of itching, and is the most common symptom in dermatology. (Reamy B, et al., American Family Physician, 84(2) 195-202 (2011), herein incorporated by reference). The severity of pruritis can vary widely, from a mild irritant to interference with work or sleep. Pruritis can be aging-associated (Reich A, et al., Clinics in Dermatology 29:15-23 (2011), herein incorporated by reference), but can also occur as a symptom for diseases found in patients of all ages. It has been defined as “an unpleasant sensation that may lead to intensive scratching.” (Id.) Pruritis can be acute (lasting less than 6 weeks) or chronic (defined as lasting more than 6 weeks) (Id.), and can be a symptom of a distinct dermatologic condition or of an occult underlying systemic disease (Reamy, supra).

Distinct dermatologic conditions (skin diseases) exhibiting pruritis include, for example, the following: xerosis, dermatitis, dyshydrotic dermatitis, drug reactions, urticaria, atopic dermatitis/neurodermatitis, seborrheic dermatitis, psoriasis, palmoplantar pustulosis, lichen planus, pityriasis rubra pilaris, darier disease, Hailey-Hailey disease, Grover’s disease, polymorphic light eruptions, bullous pemphigoid, acquired epidermolysis bullosa, dermatitis herpetiformis, pemphigus vulgaris, dermatomyositis, systemic sclerosis, Sjögren syndrome, Herpes simplex, Herpes zoster, tineas, candidal intertrigo; malassezia folliculitis, Ofuji’s disease, scabies, lice, cutaneous larva migrans, insect bites/arthropod reactions, rosacea, mastocytosis, cutaneous lymphomas, mycosis fungoides, and Sezary syndrome. (Reich, supra).

Systemic diseases that exhibit accompanying generalized pruritis include, for example: Liver diseases (primary biliary cirrhosis, primary sclerosing cholangitis, extrahepatic cholestasis, Hepatitis B and C); Kidney diseases (chronic kidney insufficiency); Hematologic diseases (polycythemia vera, Hodgkin disease, Non-Hodgkin lymphomas, leukemias, myeloma multiplex, iron deficiency, systemic mastocytosis, hypereosinophilic syndrome, myelodysplastic syndromes); Endocrine disorders (hyperthyroidism, hypothyroidism, hyperparathyroidism, diabetes); Neurologic diseases (neuropathic pruritus); Brain injury/tumor (unilateral pruritus); sclerosis multiplex; small fiber neuropathy; solid tumors (paraneoplastic pruritus); carcinoid syndrome; and infectious diseases (HIV infection/AIDS, infestations) (Id.)

Current topical therapies to treat pruritis include the following agents: cooling agents (menthol, ilicin); anesthetics (benzocaine, lidocaine, polidocanol); antihistamines (doxepin); capsaicin; corticosteroids; calcineurin inhibitors (pimecrolimus, tacrolimus); and endocannabinoids (anadamide, N-palmitoyl ethanolamine).

Current systemic therapies to treat pruritis include the following agents: antihistamines (diphenhydramine, cetirizine); opioid receptor agonists and antagonists (naltrexone, naloxone, nalfurafine); ondansetron; cholestyramine; gabapentin; pregabalin; antidepressants (paroxetine, fluvoxamine); and aprepitant.

### Xerosis

Xerosis, also known as dry skin, is a common skin disorder and affects the general population. (Barco D, et al., Actas Dermosifiliogr. 99:671-82 (2008) herein incorporated by reference in its entiretly). In clinical practice, its characteristics include rough and scaly skin, and as a disorder it exhibits rough, flaky skin that has lost normal mechanical characteristics. It is often accompanied by pruritis. (Id.) Although it can be present in healthy patients, it can also be a pathophysiologic condition, characterized by dehydration, disrupted stratum corneum, and impaired keratinocyte differentiation. (Id.) Severe xerosis can manifest itself as significantly itchy, fissured and cracked skin. Dehydration and altered lipid composition of the skin occurs when the stratum corneum can no longer retain water and has a net loss of moisture. (Id.) Xerosis can also be associated with aging, and affects three quarters of individuals over 75 years of age. (Paul C, et al., Dermatology 223:260-65 (2011)). Other factors involved in the onset of xerosis include: genetic inheritance; comorbid diseases (e.g. atopic dermatitis, psoriasis, hypothyroidism, intestinal malabsorption); temperature; humidity; sunlight exposure; seasonal conditions; air conditioning and heating; soaps/bath gels; lotions/perfumes; detergents; pharmacotherapy; friction; abrasion; and radiation. (Id.)

Pathological conditions that are associated with xerosis include: atopic dermatitis; ichthyosis; eczema; psoriasis, hypothyroidism; renal disease; malnutrition; diabetes; inflammatory disease. (Id.)

### L. Reagents, Devices, and Kits

Also provided are reagents, devices, and kits thereof for practicing one or more of the above-described methods. The subject reagents, devices, and kits thereof may vary greatly. Reagents and devices of interest include those mentioned above with respect to the methods of administering the compounds for formula 1 in the subject.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g. diskette, CD, portable flash drive, etc., on which the information has been recorded. Yet another means that may be present is a website address which may be used via the internet to access the information at a remote site. Any convenient means may be present in the kits.

### VIII. EXAMPLES

The following examples are provided by way of illustration and not by way of limitation.

### A. Pharmaceutical Preparation

The pharmaceutical compositions that are administered to subjects with symptoms of skin disorders that are comprised of the compounds, co-crystals, and salts described above can be synthesized, made, and formulated using the examples disclosed in U.S. Pat. Application Publication Nos. 2013/0266646, 2016/0081998, U.S. Pat. Nos. 8,278,302, 8,653,075, RE 45323, 8,742,115, 9,233,950, and 8,680,280, which are herein incorporated by reference in their entirety. Further, the pharmaceutical compositions may be prepared as described in the examples below:

### 1. Tablet Formulation - Wet Granulation

Copovidone is dissolved in ethanol at ambient temperature to produce a granulation liquid. An active CCR3 antagonist ingredient, lactose and part of the crospovidone are blended in a suitable mixer, to produce a pre-mix. The pre-mix is moistened with the granulation liquid and subsequently granulated. The moist granulate is optionally sieved through a sieve with a mesh size of 1.6-3.0 mm. The granulate is dried at 45° C. in a suitable dryer to a residual moisture content corresponding to 1-3 % loss on drying. The dried granulate is sieved through a sieve with a mesh size of 1.0 mm. The granulate is blended with part of the crospovidone and microcrystalline cellulose in a suitable mixer. Magnesium stearate is added to this blend after passing through a 1.0 mm sieve for delumping. Subsequently the final blend is produced by final blending in a suitable mixer and compressed into tablets. The following tablet composition can be obtained:

| Component | mg/tablet | %/tablet |
|---|---|---|
| Active ingredient | 28.500 | 30.0 |
| Crospovidone | 1.500 | 1.6 |
| Lactose | 28.000 | 29.5 |
| Copovidone | 3.000 | 3.2 |
| Total (granulate) | 61.000 | 64.3 |
| Microcrystalline cellulose | 31.000 | 32.6 |
| Crospovidone | 2.500 | 2.6 |
| Magnesium stearate | 0.500 | 0.5 |
| Total | 95.000 | 100.000 |

### 2. Tablet Formulation - Melt Granulation

An active CCR3 antagonist ingredient, lactose, part of the mcc, polyethylene glycole, lactose and part of the crospovidone are blended in a suitable mixer, to produce a pre-mix. The pre-mix is heated in a high shear mixer and subsequently granulated. The hot granulate is cooled down to room temperature and sieved through a sieve with a mesh size of 1.0 mm. The granulate is blended with part of the crospovidone and microcrystalline cellulose in a suitable mixer. Magnesium stearate is added to this blend after passing through a 1.0 mm sieve for delumping. Subsequently the final blend is produced by final blending in a suitable mixer and compressed into tablets. The following tablet composition can be obtained:

| Component | mg/tablet | %/tablet |
|---|---|---|
| Active ingredient | 28.500 | 30.0 |
| Crospovidone | 1.500 | 1.6 |
| Lactose | 11.000 | 11.6 |
| Polyethylene glycole | 14.300 | 15.1 |
| MCC | 5.700 | 6.0 |
| Total (granulate) | 61.000 | 64.3 |
| Microcrystalline cellulose | 31.000 | 32.6 |
| Crospovidone | 2.500 | 2.6 |
| Magnesium stearate | 0.500 | 0.5 |
| Total | 95.000 | 100.000 |

### 3. Tablet Formulation - Hot Melt Granulation

An active CCR3 antagonist ingredient, mannit, polyethylene glycole and part of the crospovidone are blended in a suitable mixer, to produce a pre-mix. The pre-mix is heated in a high shear mixer and subsequently granulated. The hot granulate is cooled down to room temperature and sieved through a sieve with a mesh size of 1.0 mm. The granulate is blended with part of the crospovidone and mannit in a suitable mixer. Magnesium stearate is added to this blend after passing through a 1.0 mm sieve for delumping. Subsequently the final blend is produced by final blending in a suitable mixer and compressed into tablets. The following tablet composition can be obtained:

| Component | mg/tablet | %/tablet |
|---|---|---|
| Active ingredient | 28.500 | 30.0 |
| Crospovidone | 1.500 | 1.6 |
| Mannit | 16.700 | 17.6 |
| Polyethylene glycole | 14.300 | 15.1 |
| Total (granulate) | 61.000 | 64.3 |
| Mannit | 31.000 | 32.6 |
| Crospovidone | 2.500 | 2.6 |
| Magnesium stearate | 0.500 | 0.5 |
| Total | 95.000 | 100.000 |

### 4. Tablet Formulation - Hot Melt Extrusion

An active CCR3 antagonist ingredient and stearic-palmitic acid are blended in a suitable mixer, to produce a pre-mix. The pre-mix is extruded in a twin-screw-extruder and subsequently granulated. The granulate is sieved through a sieve with a mesh size of 1.0 mm. The granulate is blended with mannit and crospovidone in a suitable mixer. Magnesium stearate is added to this blend after passing through a 1.0 mm sieve for delumping. Subsequently the final blend is produced by final blending in a suitable mixer and compressed into tablets. The following tablet composition can be obtained:

| Component | mg/tablet | %/tablet |
|---|---|---|
| Active ingredient | 28.500 | 30.0 |
| Stearic-palmitic acid | 27.500 | 28.9 |
| Total (granulate) | 56.000 | 58.9 |
| Mannit | 32.600 | 34.3 |
| Crospovidone | 5.600 | 5.9 |
| Magnesium stearate | 0.800 | 0.9 |
| Total | 95.000 | 100.000 |

### 5. Tablet Formulation - Hot Melt Extrusion

An active CCR3 antagonist ingredient and stearic-palmitic acid are blended in a suitable mixer, to produce a pre-mix. The pre-mix is extruded in a twin-screw-extruder and subsequently granulated. The granulate is sieved through a sieve with a mesh size of 1.0 mm. The granulate is directly filled into hard capsules. The following capsule composition can be obtained:

| Component | mg/tablet | %/tablet |
|---|---|---|
| Active ingredient | 70.000 | 70.0 |
| Stearic-palmitic acid | 30.000 | 30.0 |
| Total (granulate) | 100.000 | 100.0 |
| Capsule | 90.000 | - |
| Total | 190.000 | 100.000 |

### 6. Tablet Formulation - Roller Compaction

An active CCR3 antagonist ingredient, part of mannit and crospovidone and magnesium stearate are blended in a suitable mixer, to produce a pre-mix. The pre-mix is compacted with a roller compactor and subsequently granulated. Optionally, the granulate is sieved through a sieve with a mesh size of 0.8 mm. The granulate is blended with part of mannit and crospovidone in a suitable mixer. Magnesium stearate is added to this blend after passing through a 1.0 mm sieve for delumping. Subsequently the final blend is produced by final blending in a suitable mixer and compressed into tablets. The following tablet composition can be obtained:

| Component | mg/tablet | %/tablet |
|---|---|---|
| Active ingredient | 28.500 | 30.0 |
| Crospovidone | 1.400 | 1.5 |
| Mannit | 34.600 | 36.4 |
| Magnesium stearate | 0.500 | 0.5 |
| Total (granulate) | 65.000 | 68.4 |
| Mannit | 27.000 | 28.4 |
| Copovidone | 1.600 | 1.7 |
| Crospovidone | 0.950 | 1.0 |
| Magnesium stearate | 0.450 | 0.5 |
| Total | 95.000 | 100.000 |

### 7. Tablet Formulation - Roller Compaction

An active CCR3 antagonist ingredient and magnesium stearate are blended in a suitable mixer, to produce a pre-mix. The pre-mix is compacted with a roller compactor and subsequently granulated. Optionally, the granulate is sieved through a sieve with a mesh size of 0.8 mm. The granulate is blended with mannit and croscarmellose sodium in a suitable mixer. Magnesium stearate is added to this blend after passing through a 1.0 mm sieve for delumping. Subsequently the final blend is produced by final blending in a suitable mixer and compressed into tablets. The following tablet composition can be obtained:

| Component | mg/tablet | %/tablet |
|---|---|---|
| Active ingredient | 114.200 | 66.0 |
| Magnesium stearate | 1.800 | 1.0 |
| Total (granulate) | 116.000 | 67.0 |
| Mannit | 51.000 | 29.5 |
| Croscarmellose sodium | 3.500 | 2.0 |
| Magnesium stearate | 2.500 | 1.5 |
| Total | 173.000 | 100.000 |

### 8. Tablet Formulation - Roller Compaction

An active CCR3 antagonist ingredient and magnesium stearate are blended in a suitable mixer, to produce a pre-mix. The pre-mix is compacted with a roller compactor and subsequently granulated. Optionally, the granulate is sieved through a sieve with a mesh size of 0.8 mm. The granulate is blended with microcrystalline cellulose and crospovidone in a suitable mixer. Magnesium stearate is added to this blend after passing through a 1.0 mm sieve for de-lumping. Subsequently the final blend is produced by final blending in a suitable mixer and compressed into tablets. The following tablet composition can be obtained:

| Component | mg/tablet | %/tablet |
|---|---|---|
| Active ingredient | 114.200 | 66.0 |
| Magnesium stearate | 1.800 | 1.0 |
| Total (granulate) | 116.000 | 67.0 |
| MCC | 51.000 | 29.5 |
| Crospovidone | 3.500 | 2.0 |
| Magnesium stearate | 2.500 | 1.5 |
| Total | 173.000 | 100.000 |

### 9. Coated Tablet Formulation

Tablet cores according above mentioned formulations can be used to produce film-coated tablets. Hydroxypropyl methylcellulose, polyethylene glycol, talc, titanium dioxide and iron oxide are suspended in purified water in a suitable mixer at ambient temperature to produce a coating suspension. The tablet cores are coated with the coating suspension to a weight gain of about 3% to produce film-coated tablets. The following film coating composition can be obtained:

| Component | mg/tablet | %/tablet |
|---|---|---|
| Hypromellose | 2.40 | 48.0 |
| Polyethylene glycol 6000 | 0.70 | 14.0 |
| Titanium dioxide | 0.90 | 18.0 |
| Talcum | 0.90 | 18.0 |
| Iron oxide red | 0.10 | 2.0 |
| Purified water (volatile component) | -- | -- |
| Total | 5.00 | 100.0 |

### B. Drug Formulation and Administration

The investigational product of the invention (Compound 1) conforms to the following chemical structure:

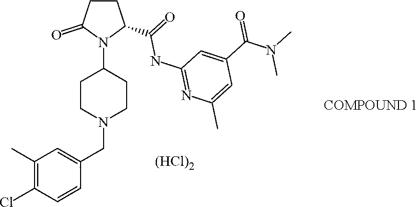

Those having ordinary skill in the relevant art would recognize that the compounds, co-crystals, salts, and formulations described previously in the sections above could also be used in these examples.

Compound 1 was made available as 100 mg, 200 mg, and 400 mg film-coated tablets with a biconvex, round or oval shape and a dull red color. The tablets were produced by a dry granulation process and contained microcrystalline cellulose, hydrogen phosphate, croscarmellose sodium, magnesium stearate, polyvinyl alcohol, titanium dioxide, polyethylene glycol, talc, iron oxide red and iron oxide yellow as inactive ingredients. Placebo tablets matching the investigational product were produced by a direct compression process and contained the same inactive ingredients.

### C. Functional Activity Examples

### 1. Inhibition of Pulmonary Eosinophil Influx

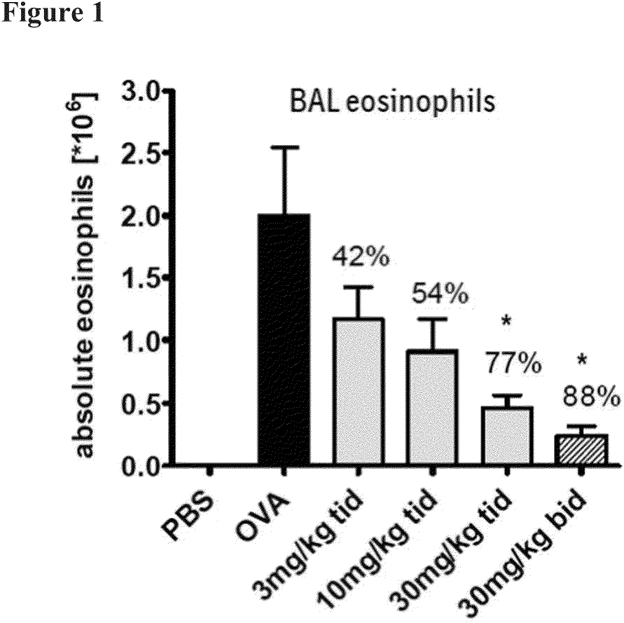

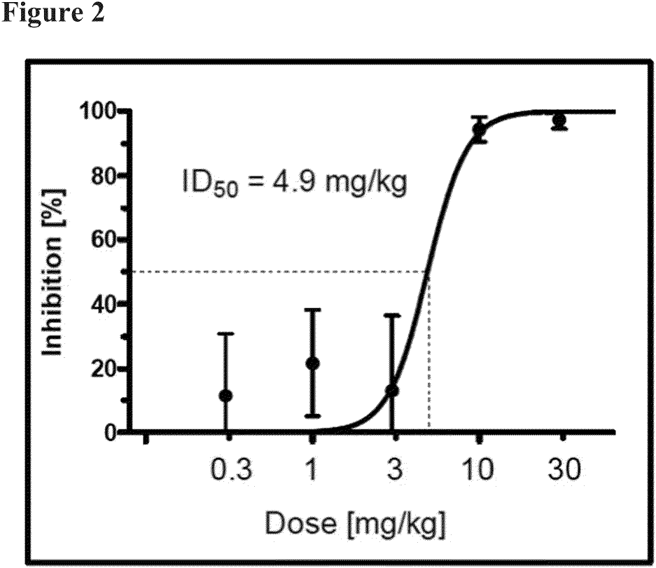

Human CCR3 knock-in BALB/c mice were sensitized with an ovalbumin/aluminum hydroxide suspension on Day 0, 4, and 8. On Day 12, Compound 1 was administered orally over a dose range from 1 to 100 mg/kg. Thirty minutes after Compound 1 administration, 3 µg of human eotaxin-1 was administered intratracheally. 4 hours after the eotaxin-1 challenge, the lungs were lavaged and the eosinophils in the bronchoalveolar lavage (BAL) fluid were counted (FIG. 1). Compound 1 dose-dependently inhibited human eotaxin-1 induced pulmonary eosinophilic inflammation with an IC₅₀ of 4.9 mg/kg (FIG. 2). These data show that Compound 1 is efficacious at decreasing pulmonary eosinophil influx in a mouse model.

### 2. Antagonistic Activity of Compound 1

Compound 1 is a potent and selective competitive antagonist of human CCR3 (Ki=3nM, 7,000 folds more selective for CCR3 vs other CCRs, t½= 18 hours). Compound 1 was validated in two human biomarker assays to show pharmacodynamic effects: (1) eosinophil shape change (ESC) and (2) CCR3 internalization.

### Eosinophil Shape Change Assay (ESC)

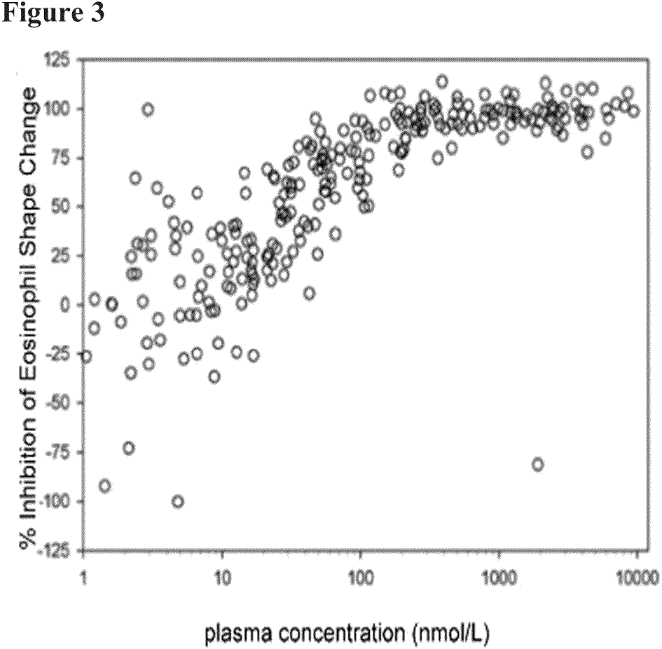

Eosinophils undergo dramatic shape changes in response to immunological and chemotactic factors (38), which can be measured in an eosinophil shape change (ESC) assay. Compound 1 inhibition of ESC was determined in whole blood samples, from Compound 1 treated patients. Whole blood samples were incubated with 30 nmol/L of eotaxin-1 at 37° C. for 7 minutes. Samples were then fixed on ice for 20 minutes followed by erythrocyte lysis at room temperature for 15 minutes. ESC was determined by flow cytometry using forward scatter (FSC) as a measure of cell size and side scatter (SSC) as a measure of granularity. The percentage inhibition of ESC was calculated and plotted (FIG. 3). Compound 1 inhibited ESC in the assay in a dose-dependent manner.

### CCR3 Internalization

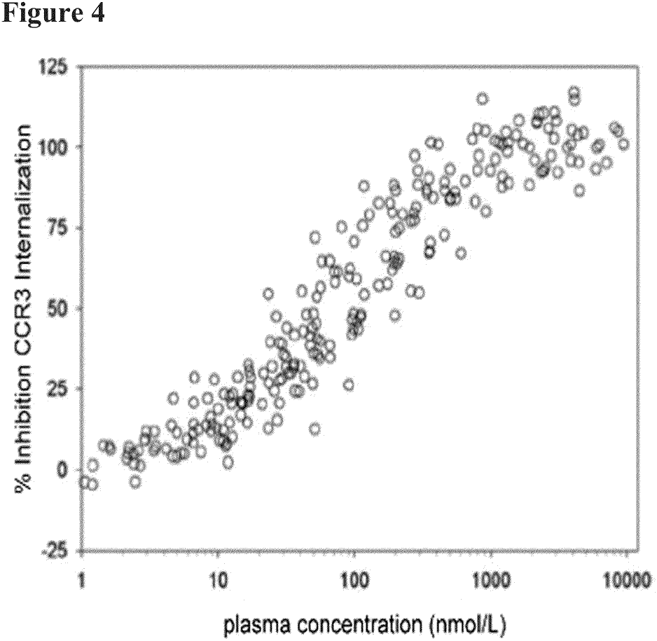

CCR3 ligand-induced internalization is a critical step in eosinophil functional response to stimulatory signals (39). Compound 1 inhibition of CCR3 internalization was determined using blood samples from patients treated with Compound 1. Whole blood samples were incubated with 30 nmol/L of eotaxin-1 at 37° C. for 30 minutes. Samples were incubated with allophycocyanin (APC)-conjugated antibody against CCR3 at room temperature in the dark for 30 minutes, followed by erythrocyte lysis and cell fixation. Samples were washed and analyzed for FSC, SSC and APC-fluorescence by flow cytometry. The percentage inhibition of CCR3 internalization was calculated and plotted (FIG. 4). Compound 1 inhibited CCR3 internalization in a dose-dependent manner.

### 3. Time-Dependent Eosinophil Infiltration in Skin and Blood With Oxazolone Model of Chronic Skin Inflammation

Eight-week-old male Hairless mice were sensitized with a topical application of 10 µL Oxazolone (at 5% wt/vol in 100% EtOH) on Day 0. The Oxazolone was administered onto the back of the mice. Starting at Day 7, the sensitized mice were treated topically every other day with 50 µL Oxazolone (at 0.1 wt/vol in 100% EtOH) on both flanks for the remainder of the study.

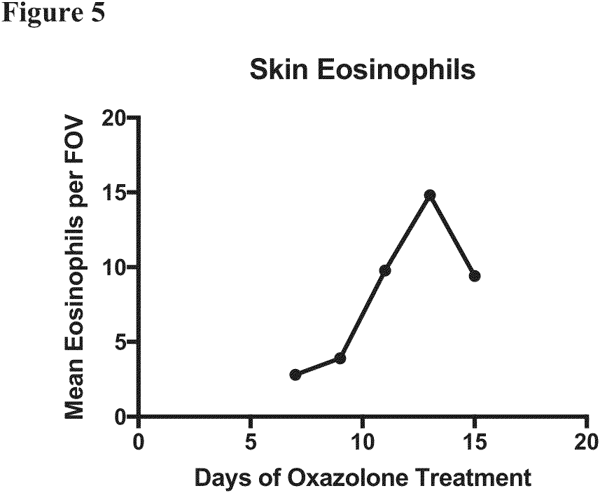

Skin eosinophils were quantified in paraffin embedded skin sections 5 µm thick, rehydrated and stained with H&E for skin morphology evaluation and by Sirius red (Sigma Aldrich 365548) for eosinophil quantification, then counted manually per field of vision, and plotted over time (FIG. 5).

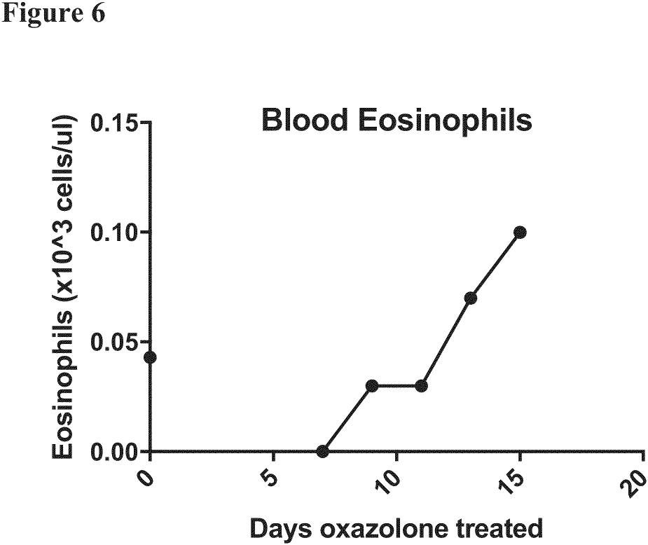

Blood eosinophils were quantified from whole blood samples from the mice, using a hematology analyzer (Advia 120), and plotted over time (FIG. 6). These experiments show that eosinophil levels in this model of chronic skin inflammation rise in a time-dependent manner.

Additional sensitization compounds or inducers that can be employed in this Model of Chronic Skin Inflammation include: 2,4,6-Trinitrochlorobenzene (TNCB), Ovalbumin, dust mite allergen, staphylococcal enterotoxin B, Imiquimod, TNCB, and Diphenylcyclopropenone. (References 21-27).

### 4. Efficacy of Compound 1 on Xerosis Using the Oxazolone Model of Chronic Skin Inflammation

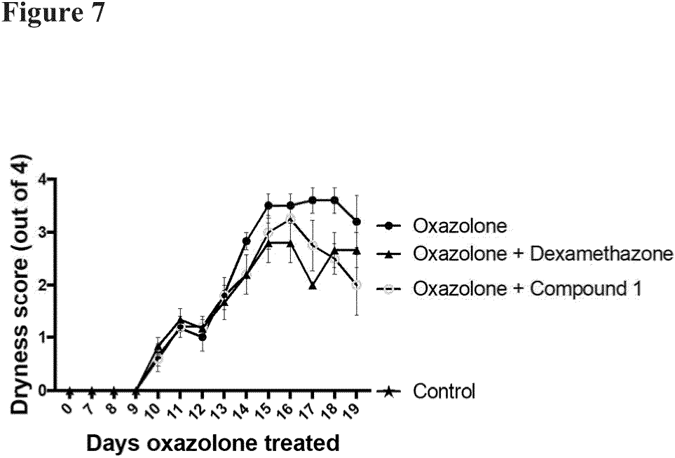

The Oxazolone model of chronic skin inflammation, was tested using Compound 1 and a known anti-inflammatory, Dexamethasone. Mice were sensitized with 10 µL of a high dose of topical Oxazolone (5% wt/vol in 100% EtOH) on their back and a subsequent chronic inflammation was triggered using low doses of topical Oxazolone (0.1% wt/vol in 100% EtOH) every other day on both flanks. Compound 1, Dexamethasone, or vehicle treatment was administrated starting at Day 0 by oral gavage (PO) (Compound 1 = 30 mg/kg b.i.d., Dexamethasone = 1.5 mg/kg q.d., vehicle = 40% HP-β-cyclodextrin pH adjusted to 6.5 with 1 M NaOH). Mice were scored daily for skin dryness/scaling using a 0-4 score ladder (where 0 represents no dryness/scaling and 4 the highest observed level of dryness/scaling). By day 17 both Compound 1 and Dexamethasone exhibited efficacy at reducing skin dryness, with Compound 1 exhibiting a trend towards greater efficacy than Dexamethasone by day 19 (FIG. 7).

### 5. Efficacy of Compound 1 on Blood Eosinophil Levels Using the Oxazolone Model of Chronic Skin Inflammation

The Oxazolone model of chronic skin inflammation, was tested using Compound 1 and a known anti-inflammatory, dexamethasone. SKH-1 Elite mice were sensitized with 10 µL of a high dose of topical Oxazolone (5% wt/vol in 100% EtOH) on their back and a subsequent chronic inflammation was triggered using low doses of topical Oxazolone (0.1% wt/vol in 100% EtOH) every other day on both flanks. Compound 1, Dexamethasone, or vehicle treatment was administrated starting at Day 0 by PO (Compound 1 = 30 mg/kg b.i.d., Dexamethasone = 1.5 mg/kg q.d., vehicle = 40% HP-β-cyclodextrin pH adjusted to 6.5 with 1 M NaOH).

Seventy to one-hundred (70-100) µL of whole blood in 10 mM EDTA freshly collected at day 29 of the study and erythrocytes were lysed in 1X lyse/fix buffer for 10 min at room temperature (558049, BD Biosciences). Unlysed cells were washed twice in PBS with 10 minutes centrifugation at 300 g between washes. Cell pellet was resuspended in 100 µL of stain buffer (554656, BD Biosciences) and incubated with 2 µL each of the following antibodies for 30 minutes: anti-SiglecF-PE (12-1702, eBioscience), anti-Ly-6G-APC (17-9668, eBioscience), anti-CD45-FITC (11-0451, eBioscience) and anti-CD11b-Super Bright 436 (62-0112, eBioscience). Cells were then washed in stain buffer, fixed in Cytofix (554655, BD Biosciences) and washed again in stain buffer with the final pellet resuspended in 300ul of stain buffer. Data were acquired on an MACSQuant Analyzer 10 (Miltenyi Biotec) with the following gating steps:
1. Debris were gated out on FSC vs SSC dotplot
2. CD45+ cells were selected on FSC vs CD45-FITC dotplot
3. CD11b high cells were selected on FSC vs CD11b-Super Bright 436 dotplot
4. Eosinophils were selected as APC- on SiglecF-PE vs Ly-6G-APC dotplot.

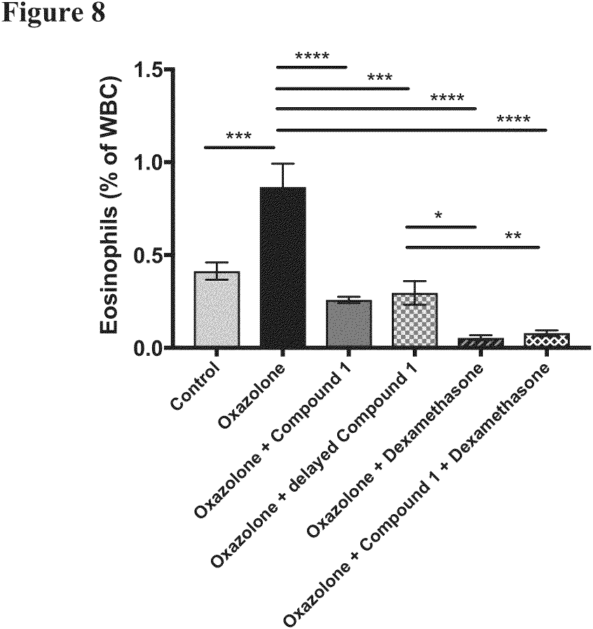

Blood eosinophil levels were quantified for each treatment group at day 29 (n=3, 6, 8, 8, 8, 6, respectively) as percentage of total measured white blood cells (WBC) (FIG. 8). All data are mean ± s.e.m. and statistical significance was assessed using one-way ANOVA followed by Tukey’s multiple comparisons test with the following adjusted p-value thresholds: <0.05 (*), < 0.01 (**), < 0.001 (***) and <0.0001 (****). Compound 1 alone returned eosinophil levels to levels similar to control mice, whereas dexamethasone resulted in a more severe reduction of eosinophil levels, indicating an overcompensating effect tied to harsher adverse effects that are a hallmark of corticosteroid therapy. Compound 1′s more nuanced effects support a more targeted approach leading to less adverse effects and better compliance.

### 6. Quantification of Blood Immune Cell Levels

The Oxazolone model of chronic skin inflammation, was tested using Compound 1 and a known anti-inflammatory, dexamethasone. SKH-1 Elite mice were sensitized with 10 µL of a high dose of topical Oxazolone (5% wt/vol in 100% EtOH) on their back and a subsequent chronic inflammation was triggered using low doses of topical Oxazolone (0.1% wt/vol in 100% EtOH) every other day on both flanks. Compound 1, Dexamethasone, or vehicle treatment was administrated starting at Day 0 by PO (Compound 1 = 30 mg/kg b.i.d., Dexamethasone = 1.5 mg/kg q.d., vehicle = 40% HP-β-cyclodextrin pH adjusted to 6.5 with 1 M NaOH).

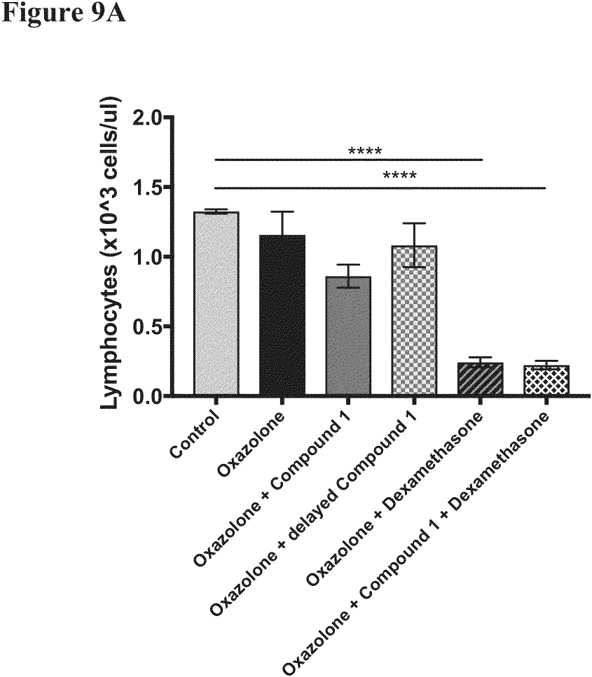

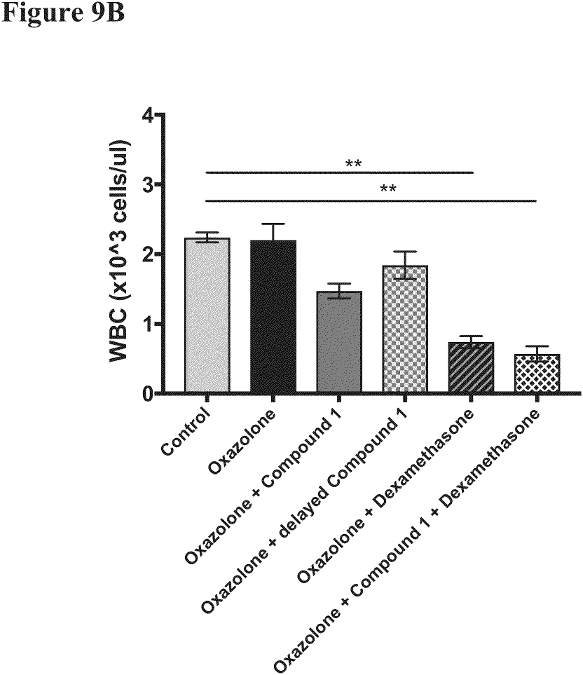

Three hundred (300) µL of whole blood in 10 mM EDTA freshly collected at day 29 of the study and erythrocytes was analyzed for counting of lymphocytes (FIG. 9A) and white blood cells (WBC) (FIG. 9B) using an Advia - 120 hematology analyzer. Lymphocyte and white blood cell (WBC) populations were quantified for each treatment group (n=3, 6, 8, 8, 8, 6, respectively). All data are mean ± s.e.m. and statistical significance was assessed using one-way ANOVA followed by Tukey’s multiple comparisons test with the following adjusted p-value thresholds: <0.05 (*), < 0.01 (**), < 0.001 (***) and <0.0001 (****). This, in conjunction with FIG. 8, shows that Compound 1 is more discriminate than dexamethasone in reduction of blood cell types levels.

### 7. Quantification of Blood Plasma Cytokines

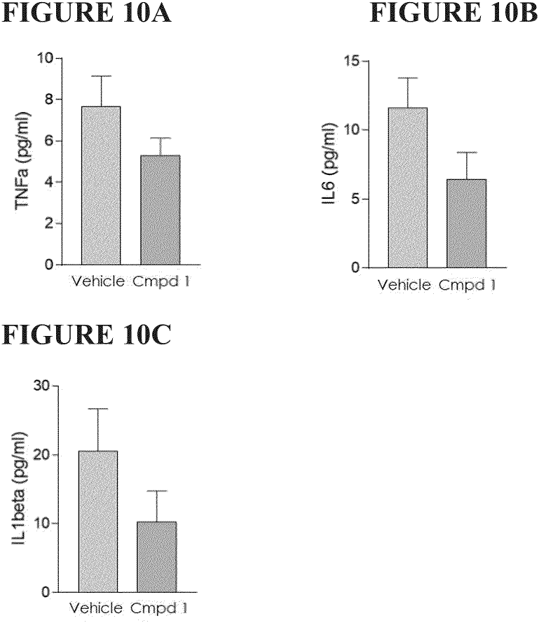

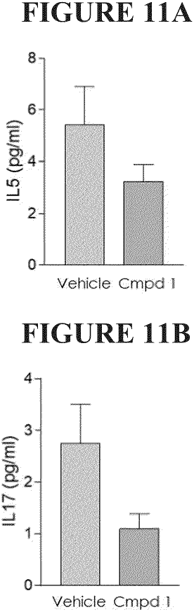

Twenty-four-month-old mice were treated PO, BID with vehicle control or Compound 1. Whole blood in 10 mM EDTA was freshly collected 2 h following treatment and centrifuged at 1,000 g for 15 minutes at 4° C. for plasma collection. This was subsequently aliquoted and stored at -80° C. Plasma levels of TNFα (FIG. 10A), IL-6 (FIG. 10B), IL-1β (FIG. 10C), IL-5 (FIG. 11A), and IL-17 (FIG. 11B) were quantified using a multiplex assay (Eve Technologies, Calgary, Canada).

Treatment with Compound 1 decreased blood plasma levels of all five cytokines. This included levels of IL-5 and IL-17 which are existing antibody targets for therapies for treating bullous pemphigoid (see Anti-IL-5 Therapy in Bullous Pemphigoid. Randomized, Placebo-controlled, Double-blind Study Evaluating the Effect of Anti-IL-5 Therapy in Patients with Bullous Pemphigoid available at world-wide-website: clinicaltrials.gov, ClinicalTrials.gov Identifier: NCT03099538; and Ixekizumab in the Treatment of Bullous Pemphigoid available at world-wide-website: clinicaltrials.gov, ClinicalTrials.gov Identifier: NCT03099538.)

### 8. Determination of Biomarkers

Biomarkers representative of the phenotypes observed using the model of Chronic Skin Inflammation discussed above are determined by evaluation over time through quantification of numerous cytokines/chemokines and other biomarkers in blood plasma and skin. Some biomarkers of interest include, for example, EDN (eosinophil derived neurotoxin), RNase3, Eotaxin-1, tumor necrosis factor (TNF), interferon gamma, IL-5, and IL-17. Evaluation and identification of biomarkers employs ELISA, Simoa and associated technologies, as well as broader proteomics approaches such as mass spectrometry and affinity-based proteomics.

Identified biomarkers are indicative of the eosinophil contribution to the symptoms (accentuating, mitigating, etc.) observed in the Chronic Skin Inflammation model. As such, they provide translational tools for clinical development.

### 9. Regimens for Treatment of Bullous Pemphigoid With Compound 1

One of skill in the art would recognize that embodiments of the invention can include treatment regimens of Compound 1 alone or in conjunction with currently approved treatments such as oral or topical steroids (e.g. Prednisone, Mometasone furoate ointment, Clobetasol propionate ointment, Betamethasone dipropionate ointment). For example, one embodiment can include administering whole body Mometasone furoate ointment (from 5-10 g or 20-25 g per day depending on severity of disease) in conjunction with Compound 1 400 mg PO, BID for a period of six weeks. Patients are monitored for blister fluid protein levels (MBP, ECP, IL-5, IL-6) at baseline and subsequent weeks. Blood eosinophil levels at baseline and at subsequent weeks are also monitored. Additionally, Anti-BP180 IgG and IgE serum levels at baseline and subsequent weeks are monitored as well as CBC, blood chemistry and plasma protein levels of MBP, ECP, IL-4, IL-5, IL1-6, IL-17A, and IFN-g). Pruritis is also monitored at baseline as well as during treatment. Another embodiment of the invention is administration of 400 mg PO, BID treatment of Compound 1 without currently approved treatments such as steroid intervention. Embodiments of the invention may also include modification of the dose of Compound 1, such as 100 mg, 200 mg, 300 mg, 400 mg, 500 mg, 600 mg, etc.

Another embodiment of the invention can include a tapering of concurrent steroid treatment over time. For example, subjects are administered 400 mg PO, BID of Compound 1 over six weeks with concurrent treatment with a steroid such as: Mometasone furoate ointment (5-10 g or 20 - 25 g per day depending on severity of bullous pemphigoid disease); Betamethasone dipropionate ointment (5-10 g or 20 - 25 g per day depending on severity of bullous pemphigoid disease); Clobetasol propionate ointment (5 g or 10 g per day depending on severity of bullous pemphigoid disease); or prednisone (0.5 mg/kg). After six weeks of Compound 1 plus steroid treatment, patients continue to receive the same dosing regimen for an additional month. After said month, the dose of the steroid regiment is tapered down. For example, the first week after the additional month, patients receive 0.4 mg/kg of prednisone plus 400 mg Compound 1 PO, BID. The second week after the additional month, patients receive 0.3 mg/kg of prednisone plus 400 mg Compound 1 PO, BID. The third week after the additional month, patients receive 0.2 mg/kg of prednisone plus 400 mg Compound 1 PO, BID. The fourth week after the additional month, patients receive 0.2 mg/kg of prednisone plus 400 mg Compound 1 PO, BID. And the fifth week after the additional month, patients stop prednisone treatment, but continue to take 400 mg Compound 1 PO, BID for a period of up to 12 months thereafter.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

What is claimed:

1. A method of treating a Bullous pemphigoid (BP) in a subject diagnosed with the skin disorder, the method comprising topically administering a therapeutically effective amount of a compound of formula 1, 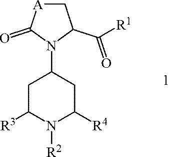
wherein
A is CH₂, O or N-C₁₋₆-alkyl;
R¹ is selected from
NHR^{1.1}, NMeR^{1.1};
NHR^{1.2}, NMeR^{1.2};
NHCH₂-R^{1.3};
NH-C₃₋₆-cycloalkyl, whereas optionally one carbon atom is replaced by a nitrogen atom, whereas the ring is optionally substituted with one or two residues selected from the group consisting of C₁₋₆-alkyl, O-C₁₋₆-alkyl, NHSO₂-phenyl, NHCONH-phenyl, halogen, CN, SO₂-C₁₋₆-alkyl, COO-C₁₋₆-alkyl;
a C₉ ₒᵣ ₁₀-bicyclic-ring, whereas one or two carbon atoms are replaced by nitrogen atoms and the ring system is bound via a nitrogen atom to the basic structure of formula 1 and whereas the ring system is optionally substituted with one or two residues selected from the group consisting of C₁₋₆-alkyl, COO-C₁₋₆-alkyl, C₁₋₆-haloalkyl, O-C₁₋₆-alkyl, NO₂, halogen, CN, NHSO₂-C₁₋₆-alkyl, methoxy-phenyl;
a group selected from NHCH(pyridinyl)CH₂COO-C₁₋₆-alkyl, NHCH(CH₂O-C₁₋₆-alkyl)-benzoimidazolyl, optionally substituted with halogen or CN;
or 1-aminocyclopentyl, optionally substituted with methyl-oxadiazole;
R^{1.1} is phenyl, optionally substituted with one or two residues selected from the group consisting of C₁₋₆-alkyl, C₂₋₆-alkenyl, C₂₋₆-alkynyl, C₁₋₆-haloalkyl, C₁₋₆-alkylene-OH, C₂₋₆-alkenylene-OH, C₂₋₆-alkynylene-OH, CH₂CON(C₁₋₆-alkyl)₂, CH₂NHCONH-C₃₋₆-cycloalkyl, CN, CO-pyridinyl, CONR^{1.1.1}R^{1.1.2}, COO-C₁₋₆-alkyl, N(SO₂-C₁₋₆-alkyl)(CH₂CON(C₁₋₄-alkyl)₂) O-C_{1 6}-alkyl, O-pyridinyl, SO₂-C₁₋₆-alkyl, SO₂-C₁₋₆-alkylen-OH, SO₂-C₃₋₆-cycloalkyl, SO₂-piperidinyl, SO₂NH-C₁₋₆-alkyl, SO₂N(C₁₋₆-alkyl)₂, halogen, CN, CO-morpholinyl, CH₂-pyridinyl or a heterocyclic ring optionally substituted with one or two residues selected from the group consisting of C₁₋₆-alkyl, NHC₁₋₆-alkyl and =O;
R^{1.1.1} H, C₁₋₆-alkyl, C₃₋₆-cycloalkyl, C₁₋₆-haloalkyl, CH₂CON(C₁₋₆-alkyl,)₂, CH₂CO-azetindinyl, C₁₋₆-alkylen-C₃₋₆-cycloalkyl, CH₂-pyranyl, CH₂-tetrahydrofuranyl, CH₂-furanyl, C₁₋₆-alkylen-OH or thiadiazolyl, optionally substituted with C₁₋₆-alkyl;
R^{1.1.2} H, C₁₋₆-alkyl, SO₂C₁₋₆-alkyl;
or R^{1.1.1} and R^{1.1.2} together are forming a four-, five- or six-membered carbocyclic ring, optionally containing one N or O, replacing a carbon atom of the ring, optionally substituted with one or two residues selected from the group consisting of C₁₋₆-alkyl, C₁₋₄-alkylene-OH, OH, ═O; or
R^{1.1} is phenyl, wherein two adjacent residues are together forming a five- or six-membered carbocyclic aromatic or non-aromatic ring, optionally containing independently from each other one or two N, S, or SO₂, replacing a carbon atom of the ring, wherein the ring is optionally substituted with C₁₋₄-alkyl or ═O;
R^{1.2} is selected from
• heteroaryl, optionally substituted with one or two residues selected from the group consisting of C₁₋₆-alkyl, C₂₋₆-alkenyl, C₂₋₆-alkynyl, C₃₋₆-cycloalkyl, CH₂COO-C₁₋₆-alkyl, CONR^{1.2.1}R^{1.2.2}, COR^{1.2.3}, COO-C₁₋₆-alkyl, CONH₂, O-C₁₋₆-alkyl, halogen, CN, SO₂N(C₁₋₆-alkyl)₂ or heteroaryl optionally substituted with one or two residues selected from the group consisting of C₁₋₆-alkyl;
heteroaryl, optionally substituted with a five- or six-membered carbocyclic non-aromatic ring containing independently from each other two N, O, S, or SO₂, replacing a carbon atom of the ring;
a aromatic or non-aromatic C₉ ₒᵣ ₁₀-bicyclic-ring, whereas one or two carbon atoms are replaced by N, O or S each optionally substituted with one or two residues selected from the group consisting of N(C₁₋₆-alkyl)₂, CONH-C₁₋₆-alkyl, ═O;
a heterocyclic non-aromatic ring, optionally substituted with pyridinyl;
4,5-dihydro-naphtho[2,1-d]thiazole, optionally substituted with NHCO-C₁₋₆-alkyl,
R^{1.2.1} H, C₁₋₆-alkyl, C₁₋₆-alkylene-C₃₋₆-cycloalkyl, C₁₋₄-alkylene-phenyl, C₁₋₄-alkylene-furanyl, C₃₋₆-cycloalkyl, C₁₋₄-alkylene-O-C₁₋₄-alkyl, C₁₋₆-haloalkyl or a five- or six-membered carbocyclic non-aromatic ring, optionally containing independently from each other one or two N, O, S, or SO₂, replacing a carbon atom of the ring, optionally substituted with 4-cyclopropylmethyl-piperazinyl
R^{1.2.2} H, C₁₋₆-alkyl;
R^{1.2.3} a five- or six-membered carbocyclic non-aromatic ring, optionally containing independently from each other one or two N, O, S, or SO₂, replacing a carbon atom of the ring;
R^{1.3} is selected from phenyl, heteroaryl or indolyl, each optionally substituted with one or two residues selected from the group consisting of C₁₋₆-alkyl, C₃₋₆-cycloalkyl, O-C₁₋₆-alkyl, O-C₁₋₆-haloalkyl, phenyl, heteroaryl;
R² is selected from the group consisting of C₁₋₆-alkylene-phenyl, C₁₋₆-alkylene-naphthyl, and C₁₋₆-alkylene-heteroaryl; each optionally substituted with one, two or three residues selected from the group consisting of C₁₋₆-alkyl, C₁₋₆-haloalkyl, O-C₁₋₆-alkyl, O-C₁₋₆-haloalkyl, halogen;
R³ is H, C₁₋₆-alkyl;
R⁴ is H, C₁₋₆-alkyl;
or R³ and R⁴ together are forming a CH₂-CH₂ group;
to treat the subject for the skin disorder.

2. (canceled)

3. The method of claim 1 wherein the compound of formula 1,
A is CH₂, O or N-C₁₋₄-alkyl;
R¹ is selected from
NHR^{1.1}, NMeR^{1.1};
NHR^{1.2}, NMeR^{1.2};
NHCH₂-R^{1.3};
NH-C₃₋₆-cycloalkyl, whereas optionally one carbon atom is replaced by a nitrogen atom, whereas the ring is optionally substituted with one or two residues selected from the group consisting of C₁₋₆-alkyl, O-C_{1 6}-alkyl, NHSO₂-phenyl, NHCONH-phenyl, halogen, CN, SO₂-C₁₋₆-alkyl, COO-C₁₋₆-alkyl;
a C₉ ₒᵣ ₁₀-bicyclic-ring, whereas one or two carbon atoms are replaced by nitrogen atoms and the ring system is bound via a nitrogen atom to the basic structure of formula 1 and whereas the ring system is optionally substituted with one or two residues selected from the group consisting of C₁₋₆-alkyl, COO-C₁₋₆-alkyl, C₁₋₆-haloalkyl, O-C₁₋₆-alkyl, NO₂, halogen, CN, NHSO₂-C₁₋₆-alkyl, m-methoxyphenyl;
a group selected from NHCH(pyridinyl)CH₂COO-C₁₋₆-alkyl, NHCH(CH₂O-C₁₋₆-alkyl)-benzoimidazolyl, optionally substituted with Cl;
or 1-aminocyclopentyl, optionally substituted with methyl-oxadiazolyl;
R^{1.1} is phenyl, optionally substituted with one or two residues selected from the group consisting of C₁₋₆-alkyl, C₁₋₆-haloalkyl, CH₂CON(C₁₋₆-alkyl)₂, CH₂NHCONH-C₃₋₆-cycloalkyl, CN, CONR^{1.1.1}R^{1.1.2}, COO-C₁₋₆-alkyl, O-C₁₋₆-alkyl, SO₂-C₁₋₆-alkyl, SO₂-C₁₋₆-alkylen-OH, SO₂-C₃₋₆-cycloalkyl, SO₂-piperidinyl, SO₂NH-C₁₋₆-alkyl, SO₂N(C₁₋₆-alkyl)₂, halogen, CN, CO-morpholinyl, CH₂-pyridinyl or a heterocyclic ring optionally substituted with one or two residues selected from the group consisting of C₁₋₆-alkyl, NHC₁₋₆-alkyl,, ═O;
R^{1.1.1} H, C₁₋₆-alkyl, C₃₋₆-cycloalkyl, C₁₋₆-haloalkyl, CH₂CON(C₁₋₆-alkyl,)₂, CH₂CO-azetindinyl, C₁₋₆-alkylen-C₃₋₆-cycloalkyl, CH₂-pyranyl, CH₂-tetrahydrofuranyl, CH₂-furanyl, C₁₋₆-alkylen-OH or thiadiazolyl, optionally substituted with C₁₋₆-alkyl;
R^{1.1.2} H, C₁₋₆-alkyl, SO₂C₁₋₆-alkyl;
or R^{1.1.1} and R^{1.1.2} together are forming a four-, five- or six-membered carbocyclic ring, optionally containing one O, replacing a carbon atom of the ring, optionally substituted with one or two residues selected from the group consisting of CH₂OH
R^{1.2} is selected from
heteroaryl, optionally substituted with one or two residues selected from the group consisting of C₁₋₆-alkyl, C₃₋₆-cycloalkyl, CH₂COO-C₁₋₆-alkyl, CONR^{1.2.1}R^{1.2.2}, COO-C₁₋₆-alkyl, CONH₂, O-C_{1 6}-alkyl, halogen, CN, CO-pyrrolidinyl, CO-morpholinyl or heteroaryl optionally substituted with one or two residues selected from the group consisting of C₁₋₆-alkyl;
benzothiazolyl, indazolyl, dihydro-indolyl, indanyl, tetrahydro-quinolinyl, each optionally substituted with one or two residues selected from the group consisting of N(C₁₋₆-alkyl)₂, CONH-C₁₋₆-alkyl, ═O;
piperidinyl, optionally substituted with pyridinyl;
4,5-dihydro-naphtho[2,1-d]thiazole, optionally substituted with NHCO-C₁₋₆-alkyl,
R^{1.2.1} H, C₁₋₆-alkyl;
R^{1.2.2} H, C₁₋₆-alkyl;
R^{1.3} is selected from phenyl, pyrazolyl, isoxazolyl, pyrimidinyl, indolyl or oxadiazolyl, each optionally substituted with one or two residues selected from the group consisting of C₁₋₆-alkyl, C₃₋₆-cycloalkyl, O-C₁₋₆-alkyl, O-C₁₋₆-haloalkyl;
R² is selected from CH₂-phenyl or CH₂-naphthyl, both optionally substituted with one or two residues selected from the group consisting of C₁₋₆-alkyl, C₁₋₆-haloalkyl, O-C₁₋₆-alkyl, O-C₁₋₆-haloalkyl, halogen; or CH₂-thiophenyl, optionally substituted with one or two residues selected from the group consisting of halogen;
R³ is H, C₁₋₄-alkyl;
R⁴ is H, C₁₋₄-alkyl;
or R³ and R⁴ together are forming a CH₂-CH₂ group.

4. The method of claim 1 wherein the compound of formula 1 is
A is CH₂, O or NMe;
R¹ is selected from
NHR^{1.1}, NMeR^{1.1};
NHR^{1.2}, NMeR^{1.2};
NHCH₂-R^{1.3};
NH-cyclohexyl, optionally substituted with one or two residues selected from the group consisting of C₁₋₄-alkyl, NHSO₂-phenyl, NHCONH-phenyl, halogen;
NH-pyrrolidinyl, optionally substituted with one or two residues selected from the group consisting of SO₂-C₁₋₄-alkyl, COO-C₁₋₄-alkyl;
piperidinyl, optionally substituted with one or two residues selected from the group consisting of NHSO₂-C₁₋₄-alkyl, m-methoxyphenyl;
dihydro-indolyl, dihydro-isoindolyl, tetrahydro-quinolinyl or tetrahydro-isoquinolinyl, optionally substituted with one or two residues selected from the group consisting of C₁₋₄-alkyl, COO-C₁₋₄-alkyl, C₁₋₄-haloalkyl, O-C₁₋₄-alkyl, NO₂, halogen;
a group selected from NHCH(pyridinyl)CH₂COO-C₁₋₄-alkyl, NHCH(CH₂O-C₁₋₄-alkyl)-benzoimidazolyl, optionally substituted with Cl;
or 1-aminocyclopentyl, optionally substituted with methyl-oxadiazolyl;
R^{1.1} is phenyl, optionally substituted with one or two residues selected from the group consisting of C₁₋₄-alkyl, C₁₋₄-haloalkyl, CH₂CON(C₁₋₄-alkyl)₂, CH₂NHCONH-C₃₋₆-cycloalkyl, CN, CONR^{1.1.1}R^{1.1.2}, COO-C₁₋₄-alkyl, O-C₁₋₄-alkyl, SO₂-C₁₋₄-alkyl, SO₂-C₁₋₄-alkylen-OH, SO₂-C₃₋₆-cycloalkyl, SO₂-piperidinyl, SO₂NH-C₁₋₄-alkyl, SO₂N(C₁₋₄-alkyl)₂, halogen, CO-morpholinyl, CH₂-pyridinyl, or imidazolidinyl, piperidinyl, oxazinanyl, pyrazolyl, triazolyl, tetrazolyl, oxazolyl, oxadiazolyl, thiazolyl, pyridinyl, pyrimidinyl, each optionally substituted with one or two residues selected from the group consisting of C₁₋₄-alkyl, NHC₁₋₄-alkyl,, ═O;
R^{1.1.1} H, C₁₋₆-alkyl, C₃₋₆-cycloalkyl, C₁₋₄-haloalkyl, CH₂CON(C₁₋₄-alkyl,)₂, CH₂CO-azetindinyl, C₁₋₄-alkylen-C₃₋₆-cycloalkyl, CH₂-pyranyl, CH₂-tetrahydrofuranyl, CH₂-furanyl, C₁₋₄-alkylen-OH or thiadiazolyl, optionally substituted with C₁₋₄-alkyl;
R^{1.1.2} H, C₁₋₄-alkyl, SO₂C₁₋₄-alkyl;
or R^{1.1.1} and R^{1.1.2} together are forming a four-, five- or six-membered carbocyclic ring, optionally containing one O, replacing a carbon atom of the ring, optionally substituted with one or two residues selected from the group consisting of CH₂OH
R^{1.2} is selected from
pyridinyl, pyridazinyl, pyrrolyl, pyrazolyl, isoxazolyl, thiazolyl, thiadiazolyl, optionally substituted with one or two residues selected from the group consisting of C₁₋₄-alkyl, C₃₋₆-cycloalkyl, CH₂COO-C₁₋₄-alkyl, CONR^{1.2.1}R^{1.2.2}, COO-C₁₋₄-alkyl, CONH₂, O-C₁₋₄-alkyl, halogen, CO-pyrrolidinyl, CO-morpholinyl or pyrazolyl, triazolyl, tetrazolyl, isoxazolyl, oxadiazolyl, each optionally substituted with one or two residues selected from the group consisting of C₁₋₄-alkyl;
benzothiazolyl, indazolyl, dihydro-indolyl, indanyl, tetrahydro-quinolinyl, each optionally substituted with one or two residues selected from the group consisting of N(C₁₋₄-alkyl)₂, CONH-C₁₋₄-alkyl, ═O;
piperidinyl, optionally substituted with pyridinyl;
4,5-dihydro-naphtho[2,1-d]thiazole, optionally substituted with NHCO-C₁₋₄-alkyl,
R^{1.2.1} H, C₁₋₄-alkyl;
R^{1.2.2} H, C₁₋₄-alkyl;
R^{1.3} is selected from phenyl, pyrazolyl, isoxazolyl, pyrimidinyl, indolyl or oxadiazolyl, each optionally substituted with one or two residues selected from the group consisting of C₁₋₄-alkyl, C₃₋₆-cycloalkyl, O-C₁₋₄-alkyl, O-C₁₋₄-haloalkyl;
R² is selected from CH₂-phenyl or CH₂-naphthyl, both optionally substituted with one or two residues selected from the group consisting of C₁₋₄-alkyl, C₁₋₄-haloalkyl, O-C₁₋₄-haloalkyl, halogen; or CH₂-thiophenyl, optionally substituted with one or two residues selected from the group consisting of halogen;
R³ is H;
R⁴ is H;
or R³ and R⁴ together are forming a CH₂-CH₂ group.

5. The method of claim 1 wherein formula 1 is
A is CH₂, O or NMe;
R¹ is selected from
NHR^{1.1}, NMeR^{1.1};
NHR^{1.2}, NMeR^{1.2};
NHCH₂-R^{1.3};
NH-piperidinyl, optionally substituted with pyridinyl;
NH-cyclohexyl, optionally substituted with one or two residues selected from the group consisting of t-Bu, NHSO₂-phenyl, NHCONH-phenyl, F;
NH-pyrrolidinyl, optionally substituted with one or two residues selected from the group consisting of SO₂Me, COO-t-Bu;
piperidinyl, optionally substituted with one or two residues selected from the group consisting of NHSO₂-n-Bu, m-methoxyphenyl;
dihydro-indolyl, dihydro-isoindolyl, tetrahydro-quinolinyl or tetrahydro-isoquinolinyl, optionally substituted with one or two residues selected from the group consisting of Me, COOMe, CF₃, OMe, NO₂, F, Br;
a group selected from NHCH(pyridinyl)CH₂COOMe, NHCH(CH₂OMe)-benzoimidazolyl, optionally substituted with Cl;
or 1-aminocyclopentyl, optionally substituted with methyl-oxadiazolyl;
R^{1.1} is phenyl, optionally substituted with one or two residues selected from the group consisting of Me, Et, t-Bu, CF₃, CH₂CONMe₂, CH₂NHCONH-cyclohexyl, CN, CONR^{1.1.1}R^{1.1.2}, COOMe, COOEt, OMe, SO₂Me, SO₂CH₂CH₂OH, SO₂Et, SO₂-cyclopropyl, SO₂-piperidinyl, SO₂NHEt, SO₂NMeEt, F, Cl, CO-morpholinyl, CH₂-pyridinyl, or imidazolidinyl, piperidinyl, oxazinanyl, pyrazolyl, triazolyl, tetrazolyl, oxazolyl, oxadiazolyl, thiazolyl, pyridinyl, pyrimidinyl, each optionally substituted with one or two residues selected from the group consisting of Me, NHMe, ═O;
R^{1.1.1} H, Me, Et, t-Bu, i-Pr, cyclopropyl, CH₂-i-Pr, CH₂-t-Bu, CH(CH₃)CH₂CH₃, CH₂CHF₂, CH₂CONMe₂, CH₂CO-azetindinyl, CH₂-cyclopropyl, CH₂-cyclobutyl, CH₂-pyranyl, CH₂-tetrahydrofuranyl, CH₂-furanyl, CH₂CH₂OH or thiadiazolyl, optionally substituted with Me;
R^{1.1.2} H, Me, Et, SO₂Me, SO₂Et
or R^{1.1.1} and R^{1.1.2} together are forming a four-, five- or six-membered carbocyclic ring, optionally containing one O, replacing a carbon atom of the ring, optionally substituted with one or two residues selected from the group consisting of CH₂OH
R^{1.2} is selected from
pyridinyl, pyrrolyl, pyrazolyl, isoxazolyl, thiazolyl, thiadiazolyl, optionally substituted with one or two residues selected from the group consisting of Me, Et, Pr, Bu, cyclopropyl, CH₂COOEt, CONR^{1.2.1}R^{1.2.2}, COOMe, COOEt, CONH₂, OMe, Cl, Br CO-pyrrolidinyl, CO-morpholinyl or pyrazolyl, triazolyl, tetrazolyl, isoxazolyl, oxadiazolyl, each optionally substituted Me;
benzothiazolyl, indazolyl, dihydro-indolyl, indanyl, tetrahydro-quinolinyl, each optionally substituted with one or two residues selected from the group consisting of NMe₂, CONHMe, ═O;
4,5-dihydro-naphtho[2,1-d]thiazole, optionally substituted with NHCOMe,
R^{1.2.1} H, Me;
R^{1.2.2} H, Me;
R^{1.3} is selected from phenyl, pyrazolyl, isoxazolyl, pyrimidinyl, indolyl or oxadiazolyl, each optionally substituted with one or two residues selected from the group consisting of Me, Et, Pr, cyclopentyl, OMe, OCHF₂;
R² is selected from CH₂-phenyl or CH₂-naphthyl, both optionally substituted with one or two residues selected from the group consisting of CH₃, CF₃, OCF₃, F, Cl, Br, Et; or CH₂-thiophenyl, optionally substituted with one or two residues selected from the group consisting of Cl, Br;
R³ is H;
R⁴ is H;
or R³ and R⁴ together are forming a CH₂-CH₂ group.

6. The method of claim 1 wherein formula 1 is
A is CH₂, O or NMe;
R¹ is selected from
NHR^{1.1}
NHR^{1.2},
R^{1.1} is phenyl, optionally substituted with one or two residues selected from the group consisting of Me, Et, Bu, CF₃, CH₂CONMe₂, CH₂NHCONH-cyclohexyl, CN, CONR^{1.1.1}R^{1.1.2}, COOMe, COOEt, OMe, SO₂Me, SO₂CH₂CH₂OH, SO₂Et, SO₂-cyclopropyl, SO₂-piperidinyl, SO₂NHEt, SO₂NMeEt, F, Cl, CO-morpholinyl, CH₂-pyridinyl, or imidazolidinyl, piperidinyl, oxazinanyl, pyrazolyl, triazolyl, tetrazolyl, oxazolyl, oxadiazolyl, thiazolyl, pyridinyl, pyrimidinyl, each optionally substituted with one or two residues selected from the group consisting of Me, NHMe, ═O;
R^{1.1.1} H, Me, Et, t-Bu, i-Pr, cyclopropyl, CH₂-i-Pr, CH₂-t-Bu, CH(CH₃)CH₂CH₃, CH₂CHF₂, CH₂CONMe₂, CH₂CO-azetindinyl, CH₂-cyclopropyl, CH₂-cyclobutyl, CH₂-pyranyl, CH₂-tetrahydrofuranyl, CH₂-furanyl, CH₂CH₂OH or thiadiazolyl, optionally substituted with Me;
R^{1.1.2} H, Me, Et, SO₂Me, SO₂Et
or R^{1.1.1} and R^{1.1.2} together are forming a four-, five- or six-membered carbocyclic ring, optionally containing one O, replacing a carbon atom of the ring, optionally substituted with one or two residues selected from the group consisting of CH₂OH
R^{1.2} is selected from
pyridinyl, pyrrolyl, pyrazolyl, isoxazolyl, thiazolyl, thiadiazolyl, optionally substituted with one or two residues selected from the group consisting of Me, Et, Pr, Bu, cyclopropyl, CH₂COOEt, CONR^{1.2.1}R^{1.2.2}, COOMe, COOEt, CONH₂, OMe, Cl, Br CO-pyrrolidinyl, CO-morpholinyl or pyrazolyl, triazolyl, tetrazolyl, isoxazolyl, oxadiazolyl, each optionally substituted Me;
benzothiazolyl, indazolyl, dihydro-indolyl, indanyl, tetrahydro-quinolinyl, each optionally substituted with one or two residues selected from the group consisting of NMe₂, CONHMe, =O;
4,5-dihydro-naphtho[2,1-d]thiazole, optionally substituted with NHCOMe,
R^{1.2.1} H, Me;
R^{1.2.2} H, Me;
R² is selected from CH₂-phenyl or CH₂-naphthyl, both optionally substituted with one or two residues selected from the group consisting of CH₃, CF₃, OCF₃, F, Cl, Br, Et
R³ is H;
R⁴ is H.

7. The method of claim 1 wherein formula 1 is
A is CH₂, O or NMe;
R¹ is selected from 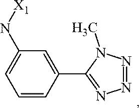 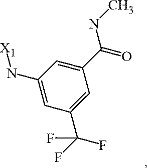 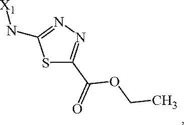 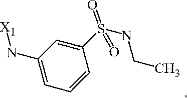 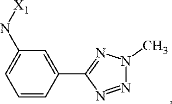 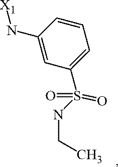 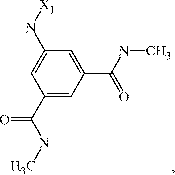 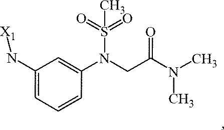 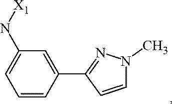 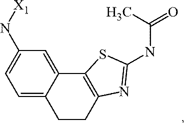 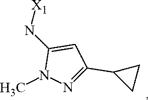 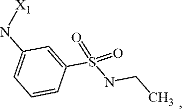 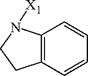 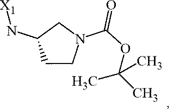 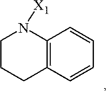 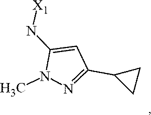 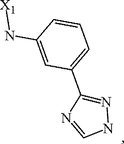 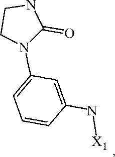 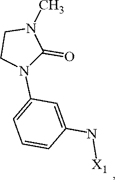 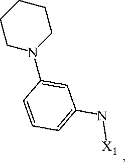 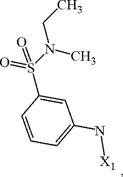 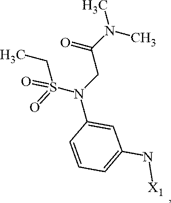 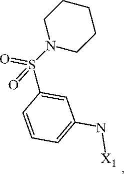 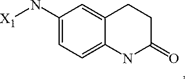 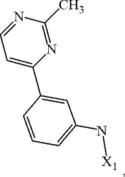 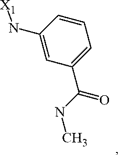 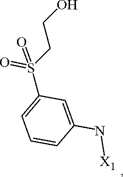 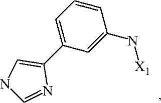 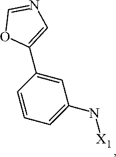 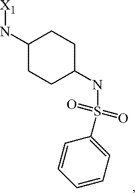 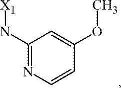 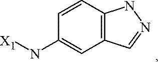 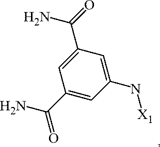 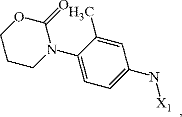 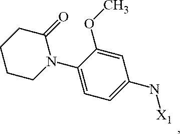 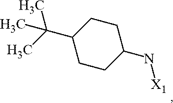 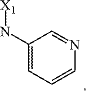 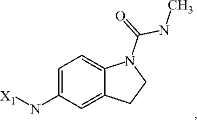 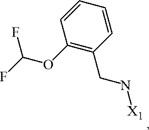 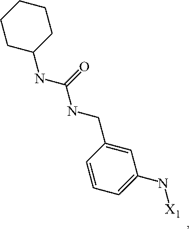 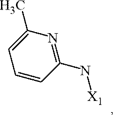 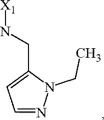 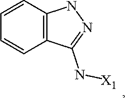 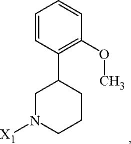 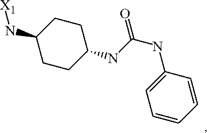 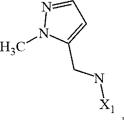 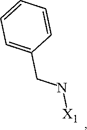 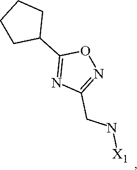 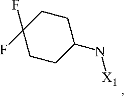 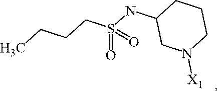 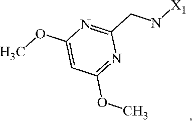 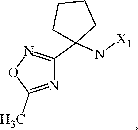 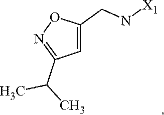 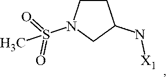 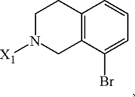 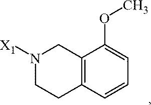 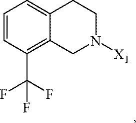 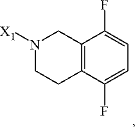 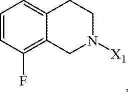 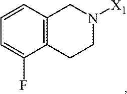 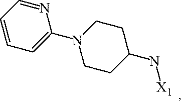 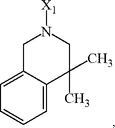 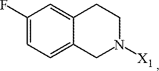 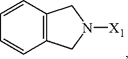 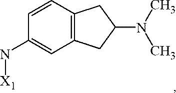 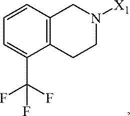 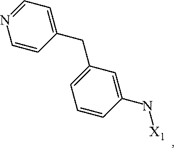 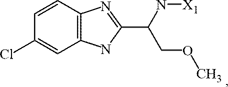 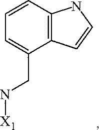 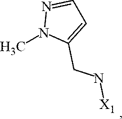 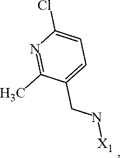 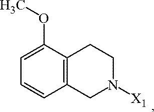 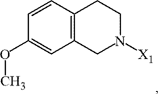 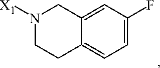 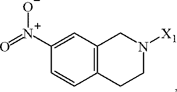 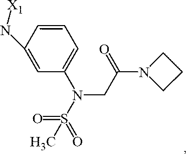 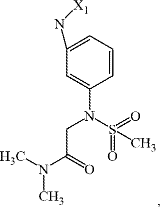 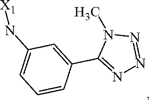 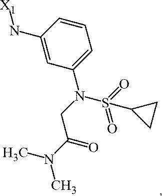 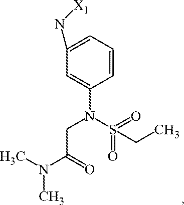 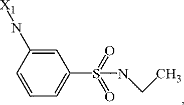 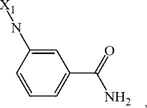 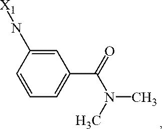 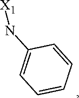 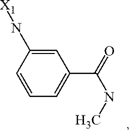 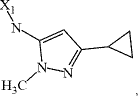 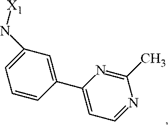 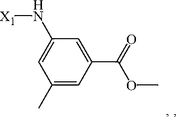 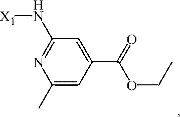 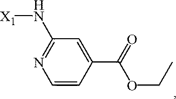 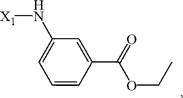 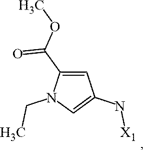 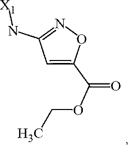 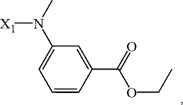 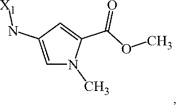 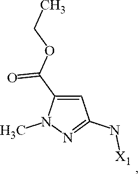 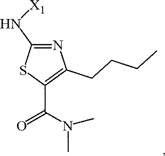 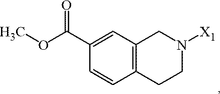 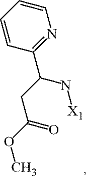 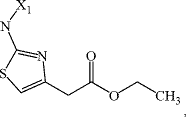 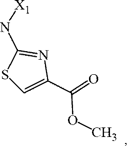 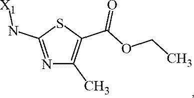 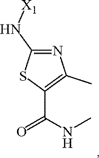 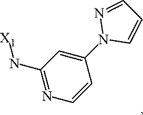 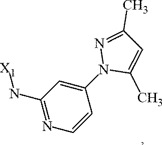 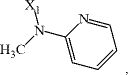 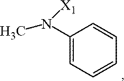 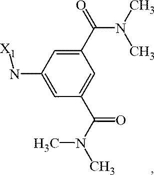 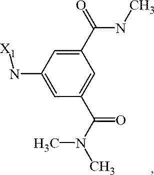 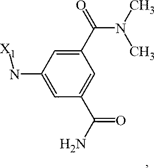 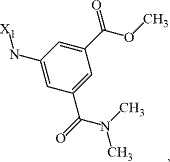 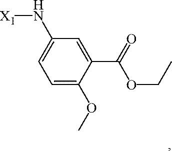 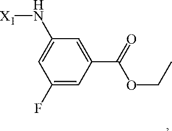 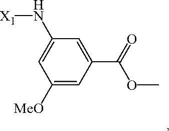 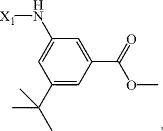 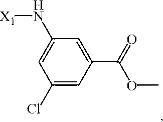 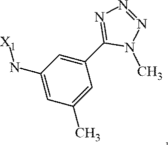 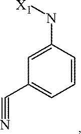 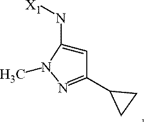 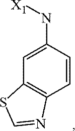 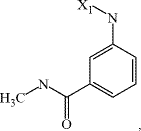 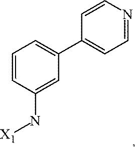 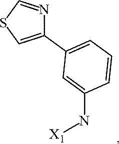 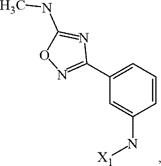 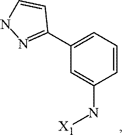 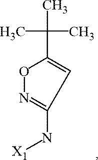 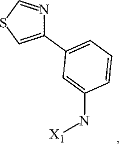 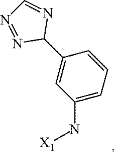 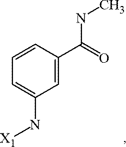 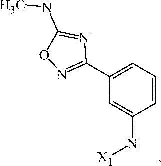 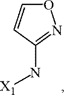 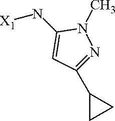 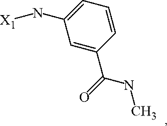 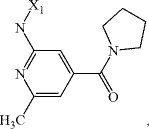 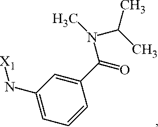 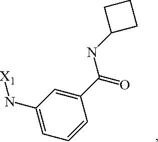 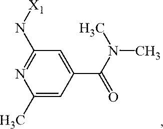 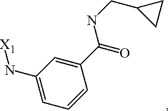 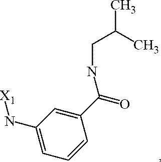 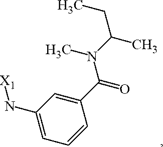 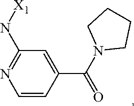 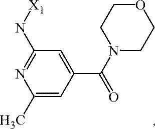 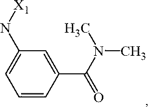 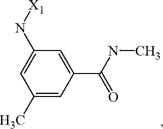 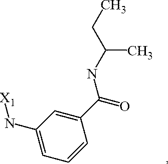 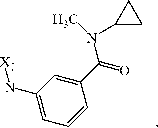 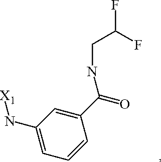 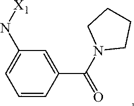 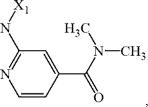 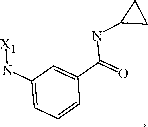 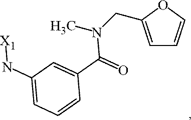 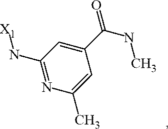 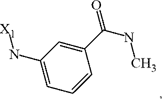 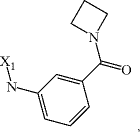 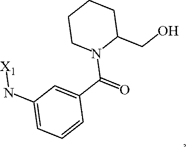 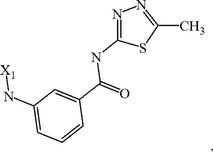 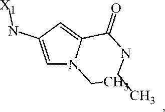 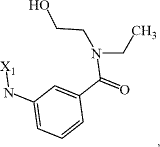 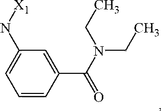 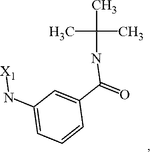 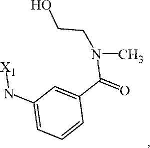 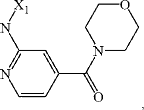 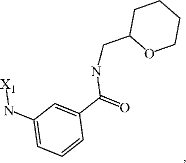 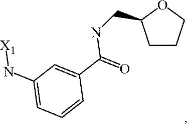 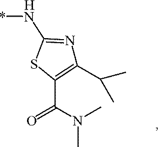 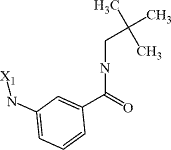 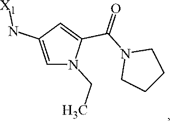 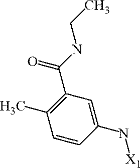 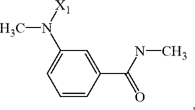 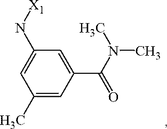 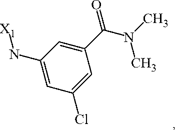 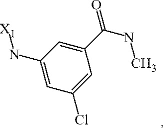 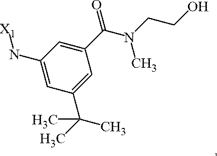 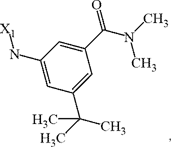 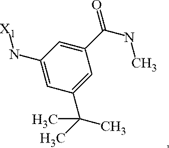 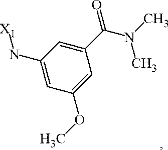 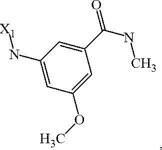 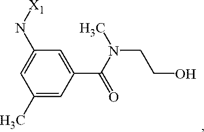 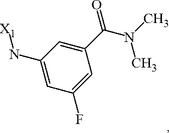 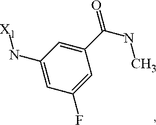 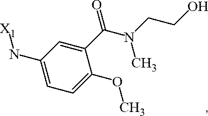 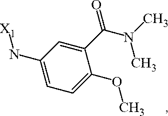 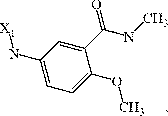 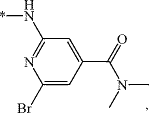 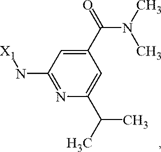 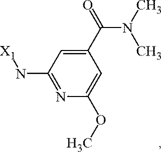 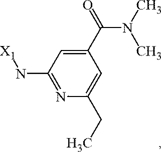 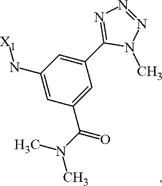 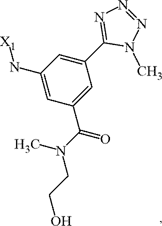 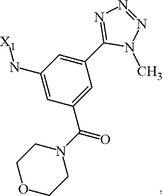 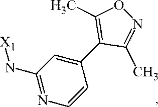 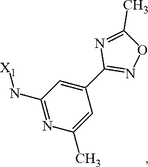 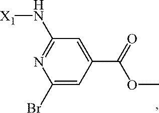 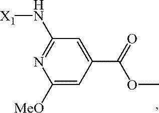 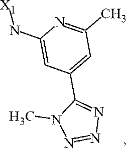 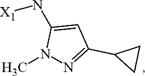 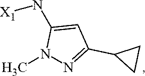 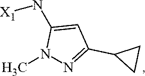 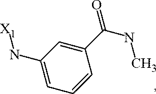 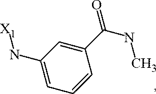 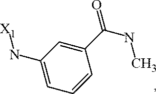 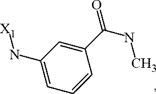 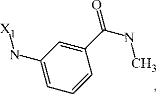 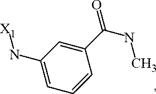 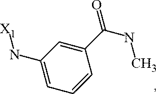 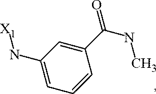 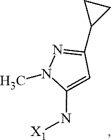 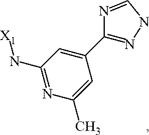 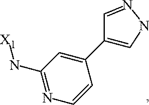 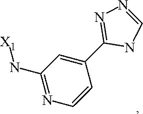 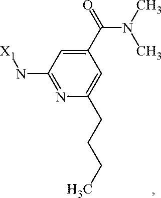 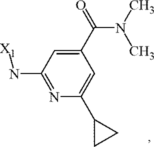 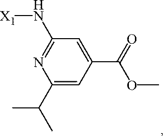 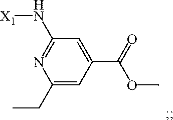
R² is selected from 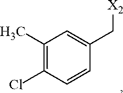 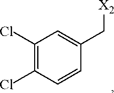 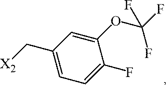 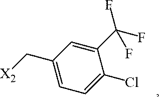 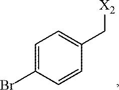 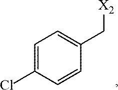 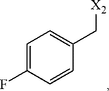 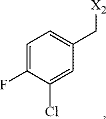 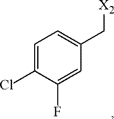 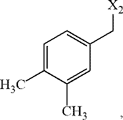 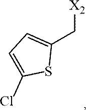 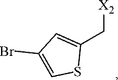 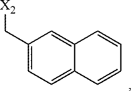 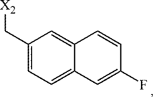 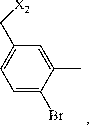
R³ is H;
R⁴ is H;
or R³ and R⁴ together are forming a CH₂-CH₂ group.

8. (canceled)

9. (canceled)

10. (canceled)

11. (canceled)

12. (canceled)

13. (canceled)

14. The method of claim 1 wherein the compound of formula 1 administered is 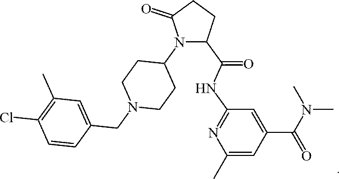
.

15. (canceled)

16. (canceled)

17. (canceled)

18. (canceled)

19. (canceled)

20. (canceled)

21. (canceled)

22. (canceled)

23. (canceled)

24. (canceled)

25. The method of claim 1 wherein the compound is a co-crystal of formula 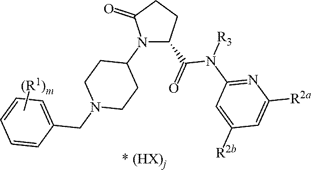
wherein
R¹ is C₁₋₆-alkyl, C₁₋₆-haloalkyl, O-C₁₋₆-haloalkyl, halogene;
m is 1, 2 or 3;
R^{2a} and R^{2b} are each independently selected from H, C₁₋₆-alkyl, C₁₋₆-alkenyl, C₁₋₆-alkynyl, C₃₋₆-cycloalkyl, COO-C₁₋₆-alkyl, O-C₁₋₆-alkyl, CONR^{2b.1}R^{2b.2}, halogene;
R^{2b.1} is H, C₁₋₆-alkyl, C₀₋₄-alkyl-C₃₋₆-cycloalkyl, C₁₋₆-haloalkyl;
R^{2b.2} is H, C₁₋₆-alkyl;
or R^{2b.1} and R^{2b.2} are together a C₃₋₆-alkylene group forming with the nitrogen atom a heterocyclic ring, wherein optionally one carbon atom or the ring is replaced by an oxygen atom
R³ is H, C₁₋₆-alkyl;
X is an anion selected from the group consisting of chloride, bromide, iodide, sulphate, phosphate, methanesulphonate, nitrate, maleate, acetate, benzoate, citrate, salicylate, fumarate, tartrate, dibenzoyltartrate, oxalate, succinate, benzoate and p-toluenesulphonate;
j is 0, 0.5, 1, 1.5 or 2;
with a co-crystal former selected from the group consisting of orotic acid, hippuric acid, L-pyroglutamic acid, D-pyroglutamic acid, nicotinic acid, L-(+)-ascorbic acid, saccharin, piperazine, 3-hydroxy-2-naphtoic acid, mucic (galactaric) acid, pamoic (embonic) acid, stearic acid, cholic acid, deoxycholic acid, nicotinamide, isonicotinamide, succinamide, uracil, L-lysine, L-proline, D-valine, L-arginine, glycine.

26. The method of claim 1 wherein the compound is a co-crystal of formula 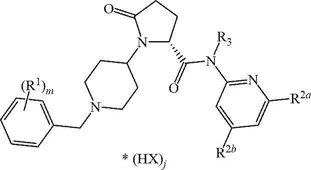
R^{2a} is H, C₁₋₆-alkyl, C₁₋₆-alkenyl, C₁₋₆-alkynyl, C₃₋₆-cycloalkyl, O-C₁₋₆-alkyl, CONR^{2a.1}R^{2a.2};
R^{2a.1} is H, C₁₋₆-alkyl, C₁₋₆-haloalkyl;
R^{2a.2} is H, C₁₋₆-alkyl;
R^{2b} is H, C₁₋₆-alkyl, C₁₋₆-alkenyl, C₁₋₆-alkynyl, C₃₋₆-cycloalkyl, COO-C₁₋₆-alkyl, O-C₁₋₆-alkyl, CONR^{2b.1}R^{2b.2}, halogene;
R^{2b.1} is H, C₁₋₆-alkyl, C₀₋₄-alkyl-C₃₋₆-cycloalkyl, C₁₋₆-haloalkyl;
R^{2b.2} is H, C₁₋₆-alkyl;
or R^{2b.1} and R^{2b.2} are together a C₃₋₆-alkylene group forming with the nitrogen atom a heterocyclic ring, wherein optionally one carbon atom or the ring is replaced by an oxygen atom.

27. The method of claim 1 wherein the compound is a co-crystal of formula 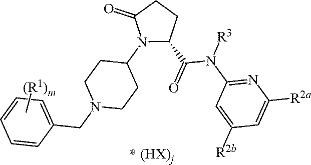
R¹ is C₁₋₆-alkyl, C₁₋₆-haloalkyl, O-C₁₋₆-haloalkyl, halogen;
m is 1 or 2;
R^{2a} is H, C₁₋₄-alkyl;
R^{2b} is H, CONR^{2b.1}R^{2b.2};
R^{2b.1} is C₁₋₄-alkyl, C₀₋₄-alkyl-C₃₋₆-cycloalkyl, C₁₋₄-haloalkyl;
R^{2b.2} is H, C₁₋₄-alkyl;
or R^{2b.1} and R^{2b.2} are together a C₃₋₆-alkylene group forming with the nitrogen atom a heterocyclic ring, wherein optionally one carbon atom or the ring is replaced by an oxygen atom
R³ is H, C₁₋₆-alkyl;
X is an anion selected from the group consisting of chloride or dibenzoyltartrate
j is 1 or 2.

28. The method of claim 1 wherein the compound is a co-crystal of formula 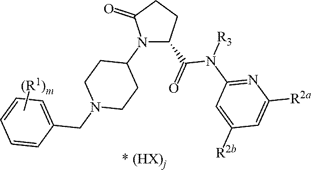
R^{2a} is H, C₁₋₄-alkyl;
R^{2b} is H, CONR^{2b.1}R^{2b.2};
R^{2b.1} is C₁₋₄-alkyl;
R^{2b.2} is C₁₋₄-alkyl.

29. The method of claim 1 wherein the compound is a co-crystal of formula 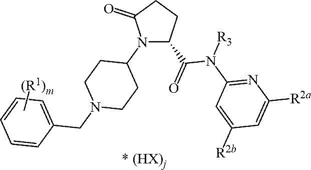
R^{2a} is H, C₁₋₄-alkyl;
R^{2b} is H, CONR^{2b.1}R^{2b.2};
R^{2b.1} is C₀₋₄-alkyl-C₃₋₆-cycloalkyl;
R^{2b.2} is H, C₁₋₄-alkyl.

30. The method of claim 1 wherein the compound is a co-crystal of formula 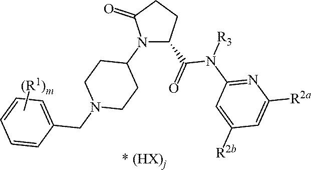
R^{2a} is H, C₁₋₄-alkyl;
R^{2b} is H, CONR^{2b.1}R^{2b.2};
R^{2b.1} is C₁₋₄-haloalkyl;
R^{2b.2} is H, C₁₋₄-alkyl.

31. The method of claim 1 wherein the compound is a co-crystal of the formula according to claim 25, wherein R^{2b.1} and R^{2b.2} are together a C₃₋₆-alkylene group forming with the nitrogen atom a heterocyclic ring, wherein optionally one carbon atom or the ring is replaced by an oxygen atom.

32. The method of claim 1 wherein the compound is a co-crystal having the formula shown below, 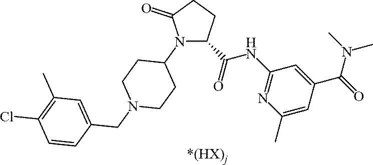
wherein j is 0,
and the co-crystal former is selected from the group consisting of L-(+)-ascorbic acid, mucic acid, pamoic acid, nicotinic acid, succinamide, nicotinamide, isonicotinamide, L-lysine, and L-proline.

33. The method of claim 1 wherein the compound is a crystalline salt of the formula below, 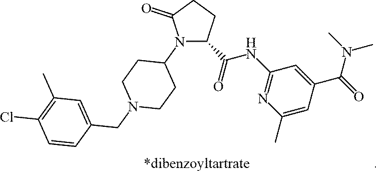
.

34. The method of claim 1 wherein the compound is a crystalline salt of the formula below, 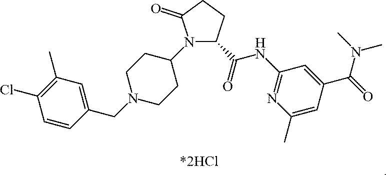
.

35. The method of claim 33 wherein the crystalline salt is characterized in that the four highest X-ray powder diffraction peaks occur at 3.72, 13.60, 16.89, and 19.34 degrees 2Θ (±0.05 degrees 2Θ) when measured using CuKα radiation.

36. The method of claim 34 wherein the crystalline salt is characterized in that the four highest X-ray powder diffraction peaks occur at 16.02, 16.86, 19,45, and 19.71 degrees 2Θ (±0.05 degrees 2Θ) when measured using CuKα radiation.

37. (canceled)

38. (canceled)

39. The method of claim 1 wherein the compound is a pharmaceutical composition comprising as an active ingredient one or more compounds of the formula below, 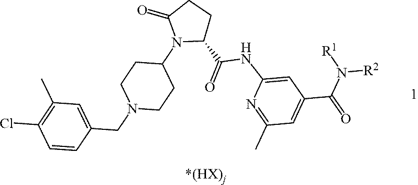
wherein
R¹ is H, C₁₋₆-alkyl, C₀₋₄-alkyl-C₃₋₆-cycloalkyl, C₁₋₆-haloalkyl;
R² is H, C₁₋₆-alkyl;
X is an anion selected from the group consisting of chloride or ½ dibenzoyltartrate
j is 1 or 2,
a first diluent, a second diluent, a binder, a disintegrant and a lubricant.

40. The method of claim 39 wherein
R¹ is H, Methyl;
R² is H, Methyl;
X is an anion selected from the group consisting of chloride or ½ dibenzoyltartrate;
j is 1 or 2.

41. The method of claim 39 wherein X is chloride and j is 2.

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)
